(19) United States
(12) Patent Application Publication
(10) Pub. No.: US 2025/0084408 A1
(43) Pub. Date: Mar. 13, 2025

(54) ENGINEERED RNAS

(71) Applicant: SHAPE THERAPEUTICS INC., Seattle, WA (US)

(72) Inventors: Susan BYRNE, Lexington, MA (US); Brian John BOOTH, Boston, MA (US); Richard Thomas SULLIVAN, Seattle, WA (US); Adrian Wrangham BRIGGS, Seattle, WA (US); Yiannis SAVVA, Seattle, WA (US)

(21) Appl. No.: 18/704,897

(22) PCT Filed: Oct. 27, 2022
(86) PCT No.: PCT/US2022/078801

§ 371 Date: Apr. 25, 2024

Related U.S. Application Data
(60) Provisional application No. 63/272,418, filed on Oct. 27, 2021.
(60) Provisional application No. 63/277,662, filed on Nov. 10, 2021.
(60) Provisional application No. 63/333,256, filed on Apr. 21, 2022.

(51) Int. Cl. C12N 15/11; C12N 15/86

(52) U.S. Cl. CPC C12N 15/11; C12N 15/86; C12N 2310/20; C12N 2310/531; C12N 2320/33; C12N 2750/14143

(57) ABSTRACT

Disclosed herein are engineered RNAs and compositions comprising the same for treatment of diseases or conditions in a subject. Also disclosed herein are methods of treating diseases or conditions in a subject by administering engineered RNAs, polynucleotides encoding the described engineered RNAs, or pharmaceutical compositions described here.

### CROSS REFERENCE

This application is a U.S. National Phase application under 35 U.S.C. § 371 of International Application Serial No. PCT/US2022/078801, filed Oct. 27, 2022, which claims priority under 35 U.S.C. § 119 from Provisional Application Ser. No. 63/272,418, filed Oct. 27, 2021, Provisional Application Ser. No. 63/277,662, filed Nov. 10, 2021, and Provisional Application Ser. No. 63/333,256, filed Apr. 21, 2022, the disclosures of which are incorporated herein by reference in their entirety.

### SEQUENCE LISTING

The instant application contains a Sequence Listing which has been submitted electronically in eXtensible Markup Language (XML) format and is hereby incorporated by reference in its entirety. Said XML copy, created on Jan. 13, 2023, is named “199235-746601_SL.xml” and is 111,766 bytes in size.

### SUMMARY

Disclosed herein is an engineered RNA, comprising: (a) a targeting sequence with complementarity to a target RNA; (b) an RNA element that comprises: (i) an engineered SmOPT variant sequence having up to 90.9% sequence identity to AAUUUGUSKAG (SEQ ID NO: 1) or AAUUUUUGGAG (SEQ ID NO: 2); and (ii) an engineered U7 hairpin variant sequence having up to 96.8% sequence identity to CAGGUUUUCUGACUUCGGUCGGAAAACCCCU (SEQ ID NO: 3) or an engineered U7 hairpin variant sequence having up to 96.9% sequence identity to UAGGCUUUCUGGCUUUUUACCGGAAAGCCCCU (SEQ ID NO: 4). In some embodiments, the engineered SmOPT variant sequence and the engineered U7 hairpin variant sequence facilitate an increase in an amount of editing of a base of a nucleotide of the target RNA by an RNA editing entity, relative to an otherwise comparable RNA lacking: the engineered SmOPT variant sequence, the engineered U7 hairpin variant sequence, or both, as determined by RNA sequencing. In some embodiments, the target RNA is associated with a disease or condition, wherein the disease or condition is selected from the group consisting of: a neurodegenerative disease or disorder, a muscular disease or disorder, a metabolic disease or disorder, an ocular disease or disorder, a liver disease or disorder, a cancer, and any combination thereof. In some embodiments, the target RNA is associated with a disease or condition selected from the group consisting of: Rett syndrome, Huntington's disease, Parkinson's Disease, Alzheimer's disease, Stargardt disease, Usher syndrome, a muscular dystrophy, Spinal Muscular Atrophy (SMN), Facioscapulohumeral Muscular Dystrophy (FSHD), Limb Girdle Muscular Dystrophy (LGMD), Amyotrophic Lateral Sclerosis (ALS), Tay-Sachs Disease, Human Immunodeficiency Virus, familial hypercholesterolemia, diabetes, and cancer. In some embodiments, the RNA element comprises the engineered SmOPT variant sequence having up to 90.9% sequence identity to SEQ ID NO: 2. In some embodiments, the 5′ end of the engineered SmOPT variant sequence comprises an added U or C relative to SEQ ID NO: 2. In some embodiments, the RNA element comprises SEQ ID NO: 39. In some embodiments, the 3′ end of the engineered SmOPT variant sequence comprises an added U or A relative to SEQ ID NO: 2. In some embodiments, nucleotides 3, 4, 5, 6 and 7 of SEQ ID NO: 2 each comprise a U, wherein nucleotide 1 is the first nucleotide of SEQ ID NO: 2 at the 5′ end. In some embodiments, the engineered SmOPT variant sequence has at least one polynucleotide substitution that comprises a G to A substitution at nucleotide 8 of SEQ ID NO: 2, wherein nucleotide 1 is the first nucleotide of SEQ ID NO: 2 at the 5′ end. In some embodiments, the engineered SmOPT variant sequence has at least one polynucleotide substitution that comprises a G to A, C, or U substitution at nucleotide 9 of SEQ ID NO: 2, wherein nucleotide 1 is the first nucleotide of SEQ ID NO: 2 at the 5′ end. In some embodiments, the engineered SmOPT variant sequence has at least one polynucleotide substitution that comprises an A to C substitution at nucleotide 10 of SEQ ID NO: 2, wherein nucleotide 1 is the first nucleotide of SEQ ID NO: 2 at the 5′ end. In some embodiments, the engineered SmOPT variant sequence has at least one polynucleotide substitution that comprises a G to A, C, or U substitution at nucleotide 11 of SEQ ID NO: 2, wherein nucleotide 1 is the first nucleotide of SEQ ID NO: 2 at the 5′ end. In some embodiments, the RNA element comprises SEQ ID NO: 40, SEQ ID NO: 41, or SEQ ID NO: 42. In some embodiments, the RNA element comprises the engineered SmOPT variant sequence having up to 90.9% sequence identity to SEQ ID NO: 1. In some embodiments, the engineered SmOPT variant sequence has at least one polynucleotide substitution that comprises a G to U substitution at nucleotide 6 of SEQ ID NO: 1, wherein nucleotide 1 is the first nucleotide of SEQ ID NO: 1 at the 5′ end. In some embodiments, the engineered SmOPT variant sequence has at least one polynucleotide substitution that comprises an A to C substitution at nucleotide 1 of SEQ ID NO: 1, wherein nucleotide 1 is the first nucleotide of SEQ ID NO: 1 at the 5′ end. In some embodiments, the engineered SmOPT variant sequence has two, three, or four polynucleotide substitutions as compared to SEQ ID NO: 1 or SEQ ID NO: 2. In some embodiments, the RNA element comprises the engineered U7 hairpin variant sequence having up to 96.8% sequence identity to SEQ ID NO: 3. In some embodiments, the engineered U7 hairpin variant sequence has at least one polynucleotide substitution that comprises a G insertion at nucleotide 3 of SEQ ID NO: 3, wherein nucleotide 1 is the first nucleotide of SEQ ID NO: 3 at the 5′ end. In some embodiments, the RNA element comprises SEQ ID NO: 44. In some embodiments, the engineered U7 hairpin variant sequence has at least one polynucleotide substitution that comprises an A to U substitution at nucleotide 2 of SEQ ID NO: 3, wherein nucleotide 1 is the first nucleotide of SEQ ID NO: 3 at the 5′ end. In some embodiments, the RNA element comprises SEQ ID NO: 43. In some embodiments, the engineered U7 hairpin variant sequence has at least one polynucleotide substitution that comprises a U to G, C, or A substitution at nucleotide 5 of SEQ ID NO: 3, wherein nucleotide 1 is the first nucleotide of SEQ ID NO: 3 at the 5′ end. In some embodiments, the RNA element comprises SEQ ID NO: 45. In some embodiments, the engineered U7 hairpin variant sequence has at least one polynucleotide substitution that comprises a U to C substitution at nucleotide 6 of SEQ ID NO: 3, wherein nucleotide 1 is the first nucleotide of SEQ ID NO: 3 at the 5′ end. In some embodiments, the RNA element comprises SEQ ID NO: 46. In some embodiments, the engineered U7 hairpin variant sequence has at least one polynucleotide substitution that comprises a U to G substitution at nucleotide 8 of SEQ ID NO: 3, wherein nucleotide 1 is the first nucleotide of SEQ ID NO: 3 at the 5′ end. In some embodiments, the engineered U7 hairpin variant sequence has at least one polynucleotide substitution that comprises a U to C or A substitution at nucleotide 10 of SEQ ID NO: 3, wherein nucleotide 1 is the first nucleotide of SEQ ID NO: 3 at the 5′ end. In some embodiments, the RNA element comprises SEQ ID NO: 47. In some embodiments, the engineered U7 hairpin variant sequence has at least one polynucleotide substitution that comprises a G to C substitution at nucleotide 11 of SEQ ID NO: 3, wherein nucleotide 1 is the first nucleotide of SEQ ID NO: 3 at the 5′ end. In some embodiments, the engineered U7 hairpin variant sequence has at least one polynucleotide substitution that comprises an A to C substitution at nucleotide 12 of SEQ ID NO: 3, wherein nucleotide 1 is the first nucleotide of SEQ ID NO: 3 at the 5′ end. In some embodiments, the RNA element comprises SEQ ID NO: 48. In some embodiments, the engineered U7 hairpin variant sequence has from two to 15 polynucleotide substitutions as compared to SEQ ID NO: 3. In some embodiments, the engineered U7 hairpin variant sequence has two, three, five, or ten polynucleotide substitutions as compared to SEQ ID NO: 3. In some embodiments, the RNA element comprises the engineered U7 hairpin variant sequence having up to 96.9% sequence identity to SEQ ID NO: 4. In some embodiments, the engineered U7 hairpin variant sequence has from two to 15 polynucleotide substitutions as compared to SEQ ID NO: 4. In some embodiments, the engineered U7 hairpin variant sequence has two, three, five, or ten polynucleotide substitutions as compared to SEQ ID NO: 4. In some embodiments, the engineered SmOPT variant sequence comprises at least one polynucleotide substitution as compared to AAUUUN₁UN₂N₃AG, wherein each of N₁, N₂, and N₃ are independently A, U, G, or C, with the proviso that: when N₁ of SEQ ID NO: 7 is G, then N₂ is A, U, or G; or N₃ is A, G, or C; or when N₁ of SEQ ID NO:7 is U, then at least one of N₂ and N₃ is A, U, or C; or when N₂ of SEQ ID NO: 7 is C, then N₁ is A, U, or C; or N₁ is A, G, or C; or when N₂ of SEQ ID NO: 7 is G, then N₁ is A, G, or C; or N₁ is A, U, or C; or when N₃ of SEQ ID NO: 7 is U, then N₁ is A, U, or C; or N₂ is A, U, or G; or when N₃ of SEQ ID NO: 7 is G, then N₁ is A, G, or C, or N₂ is A, U, or C. In some embodiments, the engineered U7 hairpin variant sequence comprises at least one polynucleotide substitution as compared to N₁AGGN₂UUUCUGN₃CUUN₄N₅N₆N₇CN₈GN₉AAAN₁₀CCCN₁₁N₁₂ (SEQ ID NO: 8), wherein each of N₁, N₂, N₃, N₄, N₅, N₆, N₇, N₈, N₉, N₁₀, N₁₁, and N₁₂ are independently A, U, G, or C, with the proviso that: when N₁ of SEQ ID NO: 8 is C, then at least one of N₂, N₇, and N₁₁ is A, G, or C; or at least one of N₃ and N₉ is U, G, or C; or at least one of N₄ and N₁₀ is A, U, or G; or at least one of N₅, N₆, and N₈ is A, U, or C; and where N₁₂ is A, U, G, C, or absent; or if N₁ of SEQ ID NO: 8 is U, then at least one of N₂, N₈, and N₁₁ is A, U, or G; or at least one of N₃, N₉, and N₁₀ is A, U, or C; or at least one of N₄, N₅, N₆, and N₁₂ is A, G, or C; or N₇ is U, G, or C; or when N₂ of SEQ ID NO: 8 is U, then at least one of N₁, N₄, and N₁₀ is A, U, or G; or at least one of N₃ and N₉ is U, G, or C; or at least one of N₅, N₆, and N₈ is A, U, or C; or at least one of N₇ and N₁₁ is A, G, or C; and N₁₂ is A, U, G, C, or absent; or if N₂ of SEQ ID NO: 8 is C, then at least one of N₁, N₄, N₅, N₆, and N₁₂ is A, G, or C; or at least one of N₃, N₉, and N₁₀ is A, U, or C; or N₇ is U, G, or C; or at least one of N₈ and N₁₁ is A, U, or G; or when N₃ of SEQ ID NO: 8 is A, then at least one of N₁, N₄, and N₁₀ is A, U, or G; or at least one of N₂, N₇, and N₁₁ is A, G, or C; or at least one of N₅, N₆, and N₈ is A, U, or C; or N₉ is U, G, or C; and N₁₂ is A, U, G, C, or absent; or if N₃ of SEQ ID NO: 8 is G, then at least one of N₁, N₄, N₅, N₆, and N₁₂ is A, G, or C; or at least one of N₂, N₈, and N₁₁ is A, U, or G; or N₇ is U, G, or C; or at least one of N₉ and N₁₀ is A, U, or C; or when N₄ of SEQ ID NO: 8 is C, then at least one of N₁ and N₁₀ is A, U, or G; or at least one of N₂, N₇, and N₁₁ is A, G, or C; or at least one of N₃ and N₉ is U, G, or C; or at least one of N₅, N₆, and N₈ is A, U, or C; and N₁₂ is A, U, G, C, or absent; or if N₄ of SEQ ID NO: 8 is U, then at least one of N₁, N₅, N₆, and N₁₂ is A, G, or C; or at least one of N₂, N₈, and N₁₁ is A, U, or G; or at least one of N₃, N₉, and N₁₀ is A, U, or C; or N₇ is U, G, or C; or when N₅ of SEQ ID NO: 8 is G, then at least one of N₁, N₄, and N₁₀ is A, U, or G; or at least one of N₂, N₇, and N₁₁ is A, G, or C; or at least one of N₃ and N₉ is U, G, or C; or at least one of N₆ and N₈ is A, U, or C; and N₁₂ is A, U, G, C, or absent; or if N₅ of SEQ ID NO: 8 is U, then at least one of N₁, N₄, N₆, and N₁₂ is A, G, or C; or at least one of N₂, N₈, and N₁₁ is A, U, or G; or at least one of N₃, N₉, and N₁₀ is A, U, or C; or N₇ is U, G, or C; or when N₆ of SEQ ID NO: 8 is G, then at least one of N₁, N₄, and N₁₀ is A, U, or G; or at least one of N₂, N₇, and N₁₁ is A, G, or C; or at least one of N₃ and N₉ is U, G, or C; or at least one of N₅ and N₈ is A, U, or C; and N₁₂ is A, U, G, C, or absent; or if N₆ of SEQ ID NO: 8 is U, then at least one of N₁, N₄, N₅, and N₁₂ is A, G, or C; or at least one of N₂, N₈, and N₁₁ is A, U, or G; or at least one of N₃, N₉, and N₁₀ is A, U, or C; or N₇ is U, G, or C; or when N₇ of SEQ ID NO: 8 is U, then at least one of N₁, N₄, and N₁₀ is A, U, or G; or at least one of N₂ and N₁₁ is A, G, or C; or at least one of N₃ and N₉ is U, G, or C; or at least one of N₅, N₆, and N₈ is A, U, or C; and N₁₂ is A, U, G, C, or absent; or if N₇ of SEQ ID NO: 8 is A, then at least one of N₁, N₄, N₅, N₆, and N₁₂ is A, G, or C; or at least one of N₂, N₈, and N₁₁ is A, U, or G; or at least one of N₃, N₉, and N₁₀; or when N₈ of SEQ ID NO: 8 is G, then at least one of N₁, N₄, and N₁₀ is A, U, or G; or at least one of N₂, N₇, and N₁₁ is A, G, or C; or at least one of N₃ and N₉ is U, G, or C; or at least one of N₅ and N₆ is A, U, or C; and N₁₂ is A, U, G, C, or absent; or if N₈ of SEQ ID NO: 8 is C, then at least one of N₁, N₄, N₅, N₆, and N₁₂ is A, G, or C; or at least one of N₂ and N₁₁ is A, U, or G; or at least one of N₃, N₉, and N₁₀ is A, U, or C; or N₇ is U, G, or C; or when N₉ of SEQ ID NO: 8 is A, then at least one of N₁, N₄, and N₁₀ is A, U, or G; or at least one of N₂, N₇, and N₁₁ is A, G, or C; or N₃ is U, G, or C; or at least one of N₅, N₆, and N₈ is A, U, or C; and N₁₂ is A, U, G, C, or absent; or if N₉ of SEQ ID NO: 8 is G, then at least one of N₁, N₄, N₅, N₆, and N₁₂ is A, G, or C; or at least one of N₂, N₈, and N₁₁ is A, U, or G; or N₇ is U, G, or C; or at least one of N₃ and N₁₀ is A, U, or C; or when N₁₀ of SEQ ID NO: 8 is C, then at least one of N₁ and N₄ is A, U, or G; or at least one of N₂, N₇, and N₁₁ is A, G, or C; or at least one of N₃ and N₉ is U, G, or C; or at least one of N₅, N₆, and N₈ is A, U, or C; and where N₁₂ is A, U, G, C, or absent; or if N₁₀ of SEQ ID NO: 8 is G, then at least one of N₁, N₄, N₅, N₆, and N₁₂ is A, G, or C; or at least one of N₂, N₈, and N₁₁ is A, U, or G; or at least one of N₃ and N₉ is A, U, or C; or N₇ is U, G, or C; or when N₁₁ of SEQ ID NO: 8 is U, then at least one of N₁, N₄, and N₁₀ is A, U, or G; or at least one of N₂ and N₇ is A, G, or C; or at least one of N₃ and N₉ is U, G, or C; or at least one of N₅, N₆, and N₈ is A, U, or C; and N₁₂ is A, U, G, C, or absent; or if N of SEQ ID NO: 8 is C, then at least one of N₁, N₄, N₅, N₆, and N₁₂ is A, G, or C; or at least one of N₂ and N₈ is A, U, or G; or at least one of N₃, N₉, and N₁₀ is A, U, or C; or N₇ is U, G, or C; or when N₁₂ of SEQ ID NO: 8 is absent, then at least one of N₁, N₄, and N₁₀ is A, U, or G; or at least one of N₂, N₇, and N₁₁ is A, G, or C; or at least one of N₃ and N₉ is U, G, or C; or at least one of N₅, N₆, and N₈ is A, U, or C; or if N₁₂ of SEQ ID NO: 8 is U, then at least one of N₁, N₄, N₅, and N₆; or at least one of N₂, N₈, and N₁₁ is A, U, or G; or at least one of N₃, N₉, and N₁₀ is A, U, or C; or N₇ is U, G, or C. In some embodiments, the RNA element comprises SEQ ID NO: 49 or SEQ ID NO: 60. In some embodiments, the RNA element comprises SEQ ID NO: 50 or SEQ ID NO: 61. In some embodiments, the RNA element comprises SEQ ID NO: 51 or SEQ ID NO: 62. In some embodiments, the targeting sequence, upon hybridization to a target RNA, forms a guide-target RNA scaffold comprising a structural feature selected from the group consisting of a mismatch, a bulge, an internal loop, a hairpin, and any combination thereof, wherein the structural feature substantially forms upon hybridization to the target RNA, and wherein the structural feature is not present within the engineered guide RNA prior to the hybridization of the engineered guide RNA to the target RNA. In some embodiments, the structural feature comprises the mismatch. In some embodiments, the mismatch comprises at least one adenosine-guanosine (A-G) mismatch, at least one adenosine-adenosine (A-A) mismatch, or at least one adenosine-cytidine (A-C), wherein adenosine is present in the target RNA. In some embodiments, the mismatch comprises an A-C mismatch, wherein the adenosine is present in the target RNA. In some embodiments, the structural feature comprises the bulge. In some embodiments, the bulge comprises an asymmetric bulge. In some embodiments, the bulge comprises a symmetric bulge. In some embodiments, the structural feature comprises the internal loop. In some embodiments, the internal loop comprises an asymmetric internal loop. In some embodiments, the internal loop comprises a symmetric internal loop. In some embodiments, the structural feature comprises the hairpin. In some embodiments, the hairpin comprises a length of about 3 bases to about 15 bases in length. In some embodiments, the engineered SmOPT variant sequence and the engineered U7 hairpin variant sequence are 3′ of the mismatch. In some embodiments, the engineered RNA is encoded by a polynucleotide that is operably linked to an RNA polymerase II-type promoter. In some embodiments, the RNA polymerase II-type promoter is selected from the group consisting of a U1 promoter, a U6 promoter, a U7 promoter, and any combination thereof. In some embodiments, the RNA polymerase II-type promoter is a U7 promoter. In some embodiments, the engineered RNA further comprises a terminator that is 3′ of the mismatch. In some embodiments, the terminator is a U7 box terminator. In some embodiments, the terminator is a truncated terminator. In some embodiments, the RNA editing entity comprises an ADAR protein. In some embodiments, the ADAR protein is selected from the group consisting of an ADAR1, an ADAR2, and any combination thereof. In some embodiments, the target RNA is selected from the group consisting of ABCA4, ALAS1, APP, ATP7B, CFTR, DMD, DMPK, DUX4, GAPDH, GBA, HEXA, HFE, LIPA, LRRK2, MAPT, PCSK9 start site, PINK1, PMP22, SERPINA1, SCNN1A start site, SNCA, or SOD1, a fragment of any one of these, and any combination thereof. In some embodiments, the target RNA is ABCA4, and wherein the ABCA4 comprises a mutation selected from the group consisting of G6320A; G5714A; G5882A; and any combination thereof. In some embodiments, the engineered RNA is configured to facilitate an edit of a base of a nucleotide of the target RNA by an RNA editing entity such that a protein translated from the edited target RNA comprises at least one amino acid residue difference as compared to a modified APP polypeptide generated from editing a base of a nucleotide of the target RNA, and wherein the at least one amino acid residue difference is selected from the group consisting of K670E, K670R, K670G, M671V, A673V, A673T, D672G, E682G, H684R, K687R, K687E, K687G, I712X, T714X of the APP polypeptide, and any combination thereof. In some embodiments, the target RNA is a SERPINA1, and wherein the SERPINA1 comprises a mutation of G9989A. In some embodiments, the target RNA is SERPINA1, and wherein the SERPINA1 encodes a mutation of E342K in a protein encoded by the target RNA. In some embodiments, the target RNA is LRRK2, and wherein the LRRK2 encodes a mutation in a protein encoded by the target RNA, where the mutation is selected from the group consisting of: E10L, A30P, S52F, E46K, A53T, L119P, A211V, C228S, E334K, N363S, V366 M, A419V, R506Q, N544E, N551K, A716V, M712V, I723V, P755L, R793 M, 1810V, K871E, Q923H, Q930R, R1067Q, S1096C, Q1111H, I1122V, A1151T, L1165P, I1192V, H1216R, S1228T, P1262A, R1325Q, I1371V, R1398H, T1410 M, D1420N, N1437H, R1441C, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141 M, R2143H, Y2189C, T2356I, G2385R, V2390 M, E2395K, M2397T, L2466H, Q2490N, and any combination thereof. In some embodiments, the target RNA is SNCA, and wherein the SNCA comprises a mutation for RNA editing selected from the group consisting of: a translation initiation site (TIS) AUG to GTG in Codon 1, a TIS AUG in Codon 5, an AUG at position 265 in Exon 2, and any combination thereof. In some embodiments, the targeting sequence has target complementarity to a splice signal proximal to an exon within the target RNA. In some embodiments, the targeting sequence: (a) has target complementarity to a branch point upstream of an exon within the target RNA; or (b) has target complementarity to a donor splice site downstream of an exon within the target RNA. In some embodiments, the mismatch is located from about 1 base to about 200 bases from either end of the targeting sequence. In some embodiments, the targeting sequence has target complementarity to a 3′ or 5′ untranslated region (UTR) of the target RNA. In some embodiments, the targeting sequence has target complementarity to a translation initiation site. In some embodiments, the targeting sequence has target complementarity to an intronic region of the target RNA. In some embodiments, the targeting sequence has target complementarity to an exonic region of the target RNA. In some embodiments, the engineered RNA is from about 80 nucleotides to about 600 nucleotides in length. In some embodiments, the engineered RNA is an antisense oligonucleotide (ASO). In some embodiments, the ASO comprises at least one chemical modification. In some embodiments, the at least one chemical modification comprises any one of: 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C₁₂ spacer, C₃ spacer, C₆ spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, sugar modified analogs, wobble/universal bases, fluorescent dye label, 2′ fluoro RNA, 2′ O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3-phosphorothioate or any combinations thereof. In some embodiments, the ASO is from about 10 nucleotides to about 200 nucleotides in length. In some embodiments, the ASO is from about 20 nucleotides to about 40 nucleotides in length. In some embodiments, the ASO is fully complementary to the target RNA. In some embodiments, the ASO is configured to inhibit, cover, mask, or block a target sequence of the target RNA. In some embodiments, the engineered RNA is a circularized engineered RNA.

Also disclosed herein is a polynucleotide encoding an engineered RNA as described herein.

Also disclosed herein is a delivery vehicle comprising an engineered RNA as described herein, or a polynucleotide encoding an engineered RNA as described herein. In some embodiments, the delivery vehicle is selected from the group consisting of a vector, a liposome, a particle, a dendrimer, and any combination thereof. In some embodiments, the delivery vehicle is a viral vector. In some embodiments, the viral vector is an adeno-associated viral (AAV) vector or derivative thereof. In some embodiments, the AAV vector, derivative thereof, or a hybrid of any of these is selected from a group consisting of: AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAVhu68, a derivative of any of these, and a hybrid of any of these. In some embodiments, the AAV vector or derivative thereof is selected from a group consisting of: a recombinant AAV (rAAV) vector, a hybrid AAV vector, a chimeric AAV vector, a self-complementary AAV (scAAV) vector, and any combination thereof.

Also disclosed herein is a pharmaceutical composition, comprising: (a) an engineered RNA as describe herein, a polynucleotide as described herein, or a delivery vehicle as described herein; and (b) a pharmaceutically acceptable: excipient, diluent, or carrier. In some embodiments, the pharmaceutical composition is in unit dose form.

Also disclosed herein is a method of treating a disease or condition in a subject, the method comprising: administering to the subject an effective amount of an engineered RNA as describe herein, a polynucleotide as described herein, a delivery vehicle as described herein; or a pharmaceutical composition as described herein to treat the disease or condition in the subject. In some embodiments, the disease or condition is selected from the group consisting of a neurodegenerative disease or disorder, a muscular disease or disorder, a metabolic disease or disorder, an ocular disease or disorder, a liver disease or disorder, a cancer, and any combination thereof. In some embodiments, the disease or condition is selected from the group consisting of Duchenne's Muscular Dystrophy (DMD), Becker muscular dystrophy, myotonic dystrophy, Facioscapulohumeral muscular dystrophy, Rett's syndrome, Charcot-Marie-Tooth disease, Alzheimer's disease, a tauopathy, Parkinson's disease, alpha-1 antitrypsin deficiency, cystic fibrosis-like disease, Wilson disease, and Stargardt's disease. In some embodiments, the disease or condition is associated with a mutation in a gene, or RNA encoded by the gene, selected from the group consisting of ABCA4, ALAS1, APP, ATP7B, CFTR, DMD, DMPK, DUX4, GAPDH, GBA, HEXA, HFE, LIPA, LRRK2, MAPT, PCSK9 start site, PINK1, PMP22, SERPINA1, SERPINA1 E342K, SCNN1A start site, SNCA, SOD1, a fragment of any of these, and any combination thereof. In some embodiments, the administering is or is by: inhalation, otic, buccal, conjunctival, dental, endocervical, endosinusial, endotracheal, enteral, epidural, extra-amniotic, extracorporeal, hemodialysis, infiltration, injection (e.g., parenchymal injection, intra-thecal injection, intra-ventricular injection, intra-cisternal injection, intravenous injection), interstitial, infraorbital, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracorneal, intracoronary, intracorporus cavernosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intranasal, intraocular, intraovarian, intrapericardial, intraperitoneal, intrapleural, intraprostatic, intrapulmonary, intrasinal, intraspinal, intraparenchymal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, stereotactic, or any combination thereof. In some embodiments, the subject is a human. In some embodiments, the subject is a subject in need thereof. In some embodiments, the subject is diagnosed with the disease or condition.

Also disclosed herein is an engineered RNA as describe herein, a polynucleotide as described herein, a delivery vehicle as described herein; or a pharmaceutical composition as described herein for use in treating a disease or condition in a subject. In some embodiments, the disease or condition is selected from the group consisting of a neurodegenerative disease or disorder, a muscular disease or disorder, a metabolic disease or disorder, an ocular disease or disorder, a liver disease or disorder, a cancer, and any combination thereof. In some embodiments, the disease or condition is selected from the group consisting of Duchenne's Muscular Dystrophy (DMD), Becker muscular dystrophy, myotonic dystrophy, Facioscapulohumeral muscular dystrophy, Rett's syndrome, Charcot-Marie-Tooth disease, Alzheimer's disease, a tauopathy, Parkinson's disease, alpha-1 antitrypsin deficiency, cystic fibrosis-like disease, Wilson disease, and Stargardt's disease. In some embodiments, the disease or condition is associated with a mutation in a gene, or RNA encoded by the gene, selected from the group consisting of ABCA4, ALAS1, APP, ATP7B, CFTR, DMD, DMPK, DUX4, GAPDH, GBA, HEXA, HFE, LIPA, LRRK2, MAPT, PCSK9 start site, PINK1, PMP22, SERPINA1, SERPINA1 E342K, SCNN1A start site, SNCA, SOD1, a fragment of any of these, and any combination thereof. In some embodiments, the administering is or is by: inhalation, otic, buccal, conjunctival, dental, endocervical, endosinusial, endotracheal, enteral, epidural, extra-amniotic, extracorporeal, hemodialysis, infiltration, injection (e.g., parenchymal injection, intra-thecal injection, intra-ventricular injection, intra-cisternal injection, intravenous injection), interstitial, infraorbital, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracorneal, intracoronary, intracorporus cavernosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intranasal, intraocular, intraovarian, intrapericardial, intraperitoneal, intrapleural, intraprostatic, intrapulmonary, intrasinal, intraspinal, intraparenchymal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, stereotactic, or any combination thereof. In some embodiments, the subject is a human.

### BRIEF DESCRIPTION OF FIGURES

Novel features of the present disclosure are set forth with particularity in the appended claims. A better understanding of the features and advantages of the present disclosure will be obtained by reference to the following detailed description that sets forth illustrative embodiments, in which exemplary principles of the present disclosure are utilized, and the accompanying drawings of which:

FIG. 1A-FIG. 1D show an exemplary mutagenesis library screen for SmOPT and U7 hairpins, highlighting single base substitutions (solid lined box), paired hairpin substitutions (dotted lined box), and extra duplicated bases (dashed lined box).

FIG. 2A shows guide RNA mutations of the SmOPT sequence with the mouse U7 (mU7) hairpin or the human U7 (hU7) hairpin sequence and the mutations effect on the fold-change editing efficiency as normalized to the unmodified SmOPT mU7 guide RNA.

FIG. 2B shows guide RNA mutations of the U1Sm sequence with the mU7 hairpin or the U7Sm sequence with the mU7 hairpin, and the mutations effect on the fold-change editing efficiency as normalized to the unmodified SmOPT mU7 guide RNA.

FIG. 2C shows mutations of the mU7 hairpin sequence with SmOPT or the hU7 hairpin sequence with SmOPT, and the mutations effect of the fold-change editing efficiency as normalized to the unmodified SmOPT mU7 guide RNA.

FIG. 2D shows the mutations in SmOPT and mU7 associated with increased editing of target RNAs. The left graphs show percent editing data from a library screen; the right graphs show percent editing data from individual single-copy transfections.

FIG. 3 shows representative individual mutations in the SmOPT and mU7 hairpin associated with increased editing of target RNAs tested in combination with each other.

FIG. 4 shows an exemplary SmOPT mU7 hairpin combined variants tested against a broader range of gene targets for RNA editing. Guide RNA expressing constructs were evaluated at Day 2 for plasmid transient transfection and Day 13 for a single-copy genomic integration.

FIG. 5 shows an exemplary SmOPT mU7 hairpin combined variants tested against exon skipping gene targets, irrespective of RNA editing. Guide RNA expressing constructs were evaluated at Day 2 for plasmid transient transfection and Day 13 for a single-copy genomic integration.

FIG. 6 shows an exemplary SmOPT mU7 hairpin combined variant tested on antisense oligonucleotides for clinically-relevant DMD exon skipping in differentiated muscle cells. Guide RNA expressing constructs were randomly integrated into the genome and evaluated after 10 days of myocyte differentiation.

### DETAILED DESCRIPTION

Disclosed herein are engineered RNAs containing RNA elements as described herein for treatment of diseases associated with mutations in a target RNA. Examples of engineered RNAs containing RNA elements useful for treatment of such diseases include engineered guide RNAs and chemically synthesized antisense oligonucleotides (ASOs). In some instances, an engineered guide RNAs can be utilized for site-specific editing of an adenosine of a target RNA using an RNA editing entity (e.g., adenosine deaminase acting on RNA (ADAR)). In some instances, an ASOs can be utilized to bind to a target RNA (blocking or covering a target RNA) to alter RNA interactions, processing, expression, or combinations thereof. As described herein, engineered RNAs of the present disclosure (e.g., engineered guide RNAs or antisense oligonucleotides (ASOs)) are operably linked to heterologous engineered RNA elements, such as a variant sequence of the Sm—or Sm-like binding domain consensus sequence (an SmOPT variant sequence), an engineered U7 hairpin variant sequence, or combinations thereof.

### RNA Elements

RNA elements of the disclosure comprise an engineered SmOPT variant sequence, an engineered U7 hairpin variant sequence, or combinations thereof as described here, where the RNA elements can be operably linked to the engineered RNAs (e.g., engineered guide RNAs, ASOs) of the description. In some embodiments of the disclosure, an RNA element of the disclosure can be an engineered SmOPT variant sequence, for example, an altered or variant of an optimized Sm or Sm-like protein binding domain, which excludes an Sm protein binding domain sequence of SEQ ID NO: 1 (AAUUUGUSKAG) or an SmOPT sequence of SEQ ID NO: 2 (AAUUUUUGGAG), and instead comprises a variant of an SmOPT sequence of SEQ ID NO: 2 (AAUUUUUGGAG) forming an engineered SmOPT variant sequence. In some embodiments, an RNA element of the disclosure can be an engineered U7 hairpin variant, which includes an altered or variant of a U7 hairpin sequence of SEQ ID NO: 3 (mouse: CAGGUUUUCUGACUUCGGUCGGAAAACCCCU) or SEQ ID NO: 4 (human: UAGGCUUUCUGGCUUUUUACCGGAAAGCCCCU) as described here.

In some embodiments, an engineered RNA (e.g., engineered guide RNA, antisense oligonucleotide) of the disclosure can comprise an RNA element, such as, an engineered SmOPT variant sequence or variant of the SmOPT sequence of SEQ ID NO: 2. As used herein, an “engineered SmOPT variant sequence” means a non-naturally occurring SmOPT sequence, a modified sequence, or a variant sequence compared to a naturally occurring sequence or an unmodified SmOPT sequence (SEQ ID NO: 2), where the engineered SmOPT variant sequence can comprise a polynucleotide substitution or at least one polynucleotide substitution (e.g., 2, 3, 4, 5, 6, 7, 8, 9, 10, 11); 11 or fewer (e.g., 10, 9, 8, 7, 6, 5, 4, 3, 2, 1); or 1-10 (e.g., 2-9, 3-8, 4-7, 5-6) as compared to a wild type, a naturally occurring, or unmodified sequence. An “engineered SmOPT variant sequence” as used herein can refer to an engineered SmOPT variant sequence AAUUUGUSKAG (SEQ ID NO: 1) that comprises an alteration of the SmOPT sequence (AAUUUUUGGAG; SEQ ID NO: 2), where the engineered SmOPT variant sequence has up to or including 90.9% sequence identity to SEQ ID NO: 2. In some embodiments, the engineered SmOPT variant sequence has at least about 9% sequence identity to SEQ ID NO: 2. In other embodiments, the engineered SmOPT variant sequence has from about 9%-90.9% sequence identity to SEQ ID NO: 2. In some embodiments, the engineered SmOPT variant sequence can comprise: at least one polynucleotide substitution (e.g., 1, 2, 3, 4, 5, 6, 7, 8, 9, 10), 10 or fewer polynucleotide substitutions (e.g., 9, 8, 7, 6, 5, 4, 3, 2, 1), or 1-10 polynucleotide substitutions, as compared to an unmodified SEQ ID NO: 2.

In some embodiments, the engineered SmOPT variant sequence can comprise the polynucleotide sequence N₀AAUUUUUGN₉AN₁₁ (SEQ ID NO: 82), wherein N₀ is absent or U and N₉ or N₁₁ is G, A, C or U. In some embodiments, the engineered SmOPT variant sequence can comprise a polynucleotide sequence with at least about: 80%, 85%, 90%, 92%, 95%, 97%, or 99% sequence identity to N₀AAUUUUUGN₉AN₁ (SEQ ID NO: 113), wherein N₀ is absent or U or A and N₉ or N₁ is G, A, C or U.

In some embodiments, the engineered SmOPT variant sequence can comprise the polynucleotide sequence N₀AAUUUUUGN₉AN₁₁ (SEQ ID NO: 82), wherein N₀ is absent or U and N₉ or N₁₁ is G, A, C or U and wherein the polynucleotide sequence is not SEQ ID NO: 2. In some embodiments, the engineered SmOPT variant sequence can comprise a polynucleotide sequence with at least about: 80%, 85%, 90%, 92%, 95%, 97%, or 99% sequence identity to N₀AAUUUUUGN₉AN₁₁ (SEQ ID NO: 82), wherein N₀ is absent or U and N₉ or N₁₁ is G, A, C or U and wherein the polynucleotide sequence is not SEQ ID NO: 2.

In some embodiments, the engineered SmOPT variant sequence can comprise the polynucleotide sequence AAUUUUUGN₉AN₁₁ (SEQ ID NO: 83), wherein N₁₁ is A, C or U. In some embodiments, the engineered SmOPT variant sequence can comprise a polynucleotide sequence with at least about: 80%, 85%, 90%, 92%, 95%, 97%, or 99% sequence identity to AAUUUUUGN₉AN₁₁ (SEQ ID NO: 83), wherein N₉ or N₁ is A, C or U.

In some embodiments, the engineered SmOPT variant sequence can comprise the polynucleotide sequence UAAUUUUUGGAG (SEQ ID NO: 84). In some embodiments, the engineered SmOPT variant sequence can comprise a polynucleotide sequence with at least about: 80%, 85%, 90%, 92%, 95%, 97%, or 99% sequence identity to UAAUUUUUGGAG (SEQ ID NO: 84).

In some embodiments, the engineered SmOPT variant sequence can comprise the polynucleotide sequence AAUUUUUGGAC (SEQ ID NO: 85). In some embodiments, the engineered SmOPT variant sequence can comprise a polynucleotide sequence with at least about: 80%, 85%, 90%, 92%, 95%, 97%, or 99% sequence identity to AAUUUUUGGAC (SEQ ID NO: 85).

In some embodiments, the engineered SmOPT variant sequence can comprise the polynucleotide sequence AAUUUUUGGAU (SEQ ID NO: 86). In some embodiments, the engineered SmOPT variant sequence can comprise a polynucleotide sequence with at least about: 80%, 85%, 90%, 92%, 95%, 97%, or 99% sequence identity to AAUUUUUGGAU (SEQ ID NO: 86).

In a preferred embodiment, the engineered SmOPT variant sequence can comprise the polynucleotide sequence AAUUUUUGGAA (SEQ ID NO: 87). In some embodiments, the engineered SmOPT variant sequence can comprise a polynucleotide sequence with at least about: 80%, 85%, 90%, 92%, 95%, 97%, or 99% sequence identity to AAUUUUUGGAA (SEQ ID NO: 87).

In some embodiments, the engineered U7 hairpin variant can comprise the polynucleotide sequence CN₂GGN₅N₆N₇UUCN₁₁GN₁₃CUUCGGN₂₀CN₂₂GAAN₂₆N₂₇CCCCN₃₂U (SEQ ID NO: 88), wherein N₂ is A or U, N₅ is either absent or G, N₆ is U or A, N₇ is U or C, N₁₁ A or U, N₁₃ is A or C, N₂₀ is U or G, N₂₂ U or G, N₂₆ is A or G, N₂₇ is A or U, and N₃₂ is either U or absent. In some embodiments, the engineered U7 hairpin variant can comprise a polynucleotide sequence with at least about: 80%, 85%, 90%, 92%, 95%, 97%, or 99% sequence identity to CN₂GGN₅N₆N₇UUCN₁₁GN₁₃CUUCGGN₂₀CN₂₂GAAN₂₆N₂₇CCCCN₃₂U (SEQ ID NO: 88), wherein N₂ is A or U, N₅ is either absent or G, N₆ is U or A, N₇ is U or C, Nu is A or U, N₁₃ is A or C, N₂₀ is U or G, N₂₂ U or G, N₂₆ is A or G, N₂₇ is A or U, and N₃₂ is either U or absent.

In some embodiments, the engineered U7 hairpin variant can comprise the polynucleotide sequence CN₂GGN₅N₆N₇UUCN₁₁GN₁₃CUUCGGN₂₀CN₂₂GAAN₂₆N₂₇CCCCN₃₂U (SEQ ID NO: 88), wherein N₂ is A or U, N₅ is either absent or G, N₆ is U or A, N₇ is U or C, N₁ is A or U, N₁₃ is A or C, N₂₀ is U or G, N₂₂ U or G, N₂₆ is A or G, N₂₇ is A or U, and N₃₂ is either U or absent and the polynucleotide sequence is not SEQ ID NO: 3. In some embodiments, the engineered U7 hairpin variant can comprise a polynucleotide sequence with at least about: 80%, 85%, 90%, 92%, 95%, 97%, or 99% sequence identity to CN₂GGN₅N₆N₇UUCN₁₁GN₁₃CUUCGGN₂₀CN₂₂GAAN₂₆N₂₇CCCCN₃₂U (SEQ ID NO: 88), wherein N₂ is A or U, N₅ is either absent or G, N₆ is U or A, N₇ is U or C, N₁ is A or U, N₁₃ is A or C, N₂₀ is U or G, N₂₂ U or G, N₂₆ is A or G, N₂₇ is A or U, and N₃₂ is either U or absent and the polynucleotide sequence is not SEQ ID NO: 3.

In some embodiments, the engineered U7 hairpin variant can comprise the polynucleotide sequence CAGGN₅UUUUCUGN₁₃CUUCGGN₂₀CGGAAAACCCCN₃₂U (SEQ ID NO: 89), wherein N₅ is either absent or G, N₁₃ is either A or C, N₂₀ is either U or G and N₃₂ is either U or absent and the polynucleotide sequence is not SEQ ID NO: 3. In some embodiments, the engineered U7 hairpin variant can comprise a polynucleotide sequence with at least about: 80%, 85%, 90%, 92%, 95%, 97%, or 99% sequence identity to CAGGN₅UUUUCUGN₁₃CUUCGGN₂₀CGGAAAACCCCN₃₂U (SEQ ID NO: 89), wherein N₅ is either absent or G, N₁₃ is either A or C, N₂₀ is either U or G and N₃₂ is either U or absent and the polynucleotide sequence is not SEQ ID NO: 3.

In some embodiments, the engineered U7 hairpin variant can comprise the polynucleotide sequence CUGGUUUUCUGACUUCGGUCGGAAAACCCCU (SEQ ID NO: 90). In some embodiments, the engineered U7 hairpin variant can comprise a polynucleotide sequence with at least about: 80%, 85%, 90%, 92%, 95%, 97%, or 99% sequence identity to CUGGUUUUCUGACUUCGGUCGGAAAACCCCU (SEQ ID NO: 90).

In some embodiments, the engineered U7 hairpin variant can comprise the polynucleotide sequence CAGGGUUUUCUGACUUCGGUCGGAAAACCCCCU (SEQ ID NO: 91). In some embodiments, the engineered U7 hairpin variant can comprise a polynucleotide sequence with at least about: 80%, 85%, 90%, 92%, 95%, 97%, or 99% sequence identity to CAGGGUUUUCUGACUUCGGUCGGAAAACCCCCU (SEQ ID NO: 91).

In some embodiments, the engineered U7 hairpin variant can comprise the polynucleotide sequence CAGGAUUUCUGACUUCGGUCGGAAAUCCCCU (SEQ ID NO: 92). In some embodiments, the engineered U7 hairpin variant can comprise a polynucleotide sequence with at least about: 80%, 85%, 90%, 92%, 95%, 97%, or 99% sequence identity to CAGGAUUUCUGACUUCGGUCGGAAAUCCCCU (SEQ ID NO: 92).

In some embodiments, the engineered U7 hairpin variant can comprise the polynucleotide sequence CAGGUCUUCUGACUUCGGUCGGAAGACCCCU (SEQ ID NO: 93). In some embodiments, the engineered U7 hairpin variant can comprise a polynucleotide sequence with at least about: 80%, 85%, 90%, 92%, 95%, 97%, or 99% sequence identity to CAGGUCUUCUGACUUCGGUCGGAAGACCCCU (SEQ ID NO: 93).

In some embodiments, the engineered U7 hairpin variant can comprise the polynucleotide sequence CAGGUUUUCAGACUUCGGUCUGAAAACCCCU (SEQ ID NO: 94). In some embodiments, the engineered U7 hairpin variant can comprise a polynucleotide sequence with at least about: 80%, 85%, 90%, 92%, 95%, 97%, or 99% sequence identity to CAGGUUUUCAGACUUCGGUCUGAAAACCCCU (SEQ ID NO: 94).

In some embodiments, the engineered U7 hairpin variant can comprise the polynucleotide sequence CAGGUUUUCUGCCUUCGGGCGGAAAACCCCU (SEQ ID NO: 95). In some embodiments, the engineered U7 hairpin variant can comprise a polynucleotide sequence with at least about: 80%, 85%, 90%, 92%, 95%, 97%, or 99% sequence identity to CAGGUUUUCUGCCUUCGGGCGGAAAACCCCU (SEQ ID NO: 95).

In some embodiments, the engineered U7 hairpin variant can comprise the polynucleotide sequence CAGGGUUUUCUGCCUUCGGGCGGAAAACCCCCU (SEQ ID NO: 96). In some embodiments, the engineered U7 hairpin variant can comprise a polynucleotide sequence with at least about: 80%, 85%, 90%, 92%, 95%, 97%, or 99% sequence identity to CAGGGUUUUCUGCCUUCGGGCGGAAAACCCCCU (SEQ ID NO: 96).

In some embodiments, the engineered U7 hairpin variant can comprise the polynucleotide sequence CAGGGUUUUCAGACUUCGGUCUGAAAACCCCCU (SEQ ID NO: 97). In some embodiments, the engineered U7 hairpin variant can comprise a polynucleotide sequence with at least about: 80%, 85%, 90%, 92%, 95%, 97%, or 99% sequence identity to CAGGGUUUUCAGACUUCGGUCUGAAAACCCCCU (SEQ ID NO: 97).

In some embodiments, the engineered U7 hairpin variant can comprise the polynucleotide sequence CAGGUUUUCAGCCUUCGGGCUGAAAACCCCU (SEQ ID NO: 98). In some embodiments, the engineered U7 hairpin variant can comprise a polynucleotide sequence with at least about: 80%, 85%, 90%, 92%, 95%, 97%, or 99% sequence identity to CAGGUUUUCAGCCUUCGGGCUGAAAACCCCU (SEQ ID NO: 98).

In some embodiments, the engineered U7 hairpin variant can comprise the polynucleotide sequence CAGGGUUUUCAGCCUUCGGGCUGAAAACCCCCU (SEQ ID NO: 99). In some embodiments, the engineered U7 hairpin variant can comprise a polynucleotide sequence with at least about: 80%, 85%, 90%, 92%, 95%, 97%, or 99% sequence identity to CAGGGUUUUCAGCCUUCGGGCUGAAAACCCCCU (SEQ ID NO: 99).

In some embodiments, the RNA element can comprise an engineered SmOPT variant having a polynucleotide sequence of SEQ ID NO: 82 and an engineered U7 hairpin variant sequence having a polynucleotide sequence of any one of SEQ ID Nos: 88 to 99.

In some embodiments, the RNA element can comprise an engineered SmOPT variant having a polynucleotide sequence of SEQ ID NO: 83 and an engineered U7 hairpin variant sequence having a polynucleotide sequence of any one of SEQ ID Nos: 88 to 99.

In some embodiments, the RNA element can comprise an engineered SmOPT variant having a polynucleotide sequence of SEQ ID NO: 84 and an engineered U7 hairpin variant sequence having a polynucleotide sequence of any one of SEQ ID Nos: 88 to 99.

In some embodiments, the RNA element can comprise an engineered SmOPT variant having a polynucleotide sequence of SEQ ID NO: 85 and an engineered U7 hairpin variant sequence having a polynucleotide sequence of any one of SEQ ID Nos: 88 to 99.

In some embodiments, the RNA element can comprise an engineered SmOPT variant having a polynucleotide sequence of SEQ ID NO: 86 and an engineered U7 hairpin variant sequence having a polynucleotide sequence of any one of SEQ ID Nos: 88 to 99.

In some embodiments, the RNA element can comprise an engineered SmOPT variant having a polynucleotide sequence of SEQ ID NO: 87 and an engineered U7 hairpin variant sequence having a polynucleotide sequence of any one of SEQ ID Nos: 88 to 99.

In some embodiments, the RNA element can comprise an engineered SmOPT variant sequence and an engineered U7 hairpin variant sequence having a polynucleotide sequence (expressed as DNA) of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 59. In some embodiments, the RNA element can comprise an engineered SmOPT variant sequence and an engineered U7 hairpin variant sequence having a polynucleotide sequence (expressed as RNA) of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, or SEQ ID NO: 70.

In some embodiments, the RNA element can comprise an engineered SmOPT variant sequence and an engineered U7 hairpin variant sequence having a polynucleotide sequence (expressed as DNA) of SEQ ID NO: 49. In some embodiments, the RNA element can comprise an engineered SmOPT variant sequence and an engineered U7 hairpin variant sequence having a polynucleotide sequence (expressed as DNA) with at least about: 80%, 85%, 90%, 92%, 95%, 97%, or 99% sequence identity to SEQ ID NO: 49.

In some embodiments, the RNA element can comprise an engineered SmOPT variant sequence and an engineered U7 hairpin variant sequence having a polynucleotide sequence (expressed as DNA) of SEQ ID NO: 50. In some embodiments, the RNA element can comprise an engineered SmOPT variant sequence and an engineered U7 hairpin variant sequence having a polynucleotide sequence (expressed as DNA) with at least about: 80%, 85%, 90%, 92%, 95%, 97%, or 99% sequence identity to SEQ ID NO: 50.

In some embodiments, the RNA element can comprise an engineered SmOPT variant sequence and an engineered U7 hairpin variant sequence having a polynucleotide sequence (expressed as DNA) of SEQ ID NO: 51. In some embodiments, the RNA element can comprise an engineered SmOPT variant sequence and an engineered U7 hairpin variant sequence having a polynucleotide sequence (expressed as DNA) with at least about: 80%, 85%, 90%, 92%, 95%, 97%, or 99% sequence identity to SEQ ID NO: 51.

In some embodiments, the RNA element can comprise an engineered SmOPT variant sequence and an engineered U7 hairpin variant sequence having a polynucleotide sequence (expressed as RNA) of SEQ ID NO: 60. In some embodiments, the RNA element can comprise an engineered SmOPT variant sequence and an engineered U7 hairpin variant sequence having a polynucleotide sequence (expressed as RNA) with at least about: 80%, 85%, 90%, 92%, 95%, 97%, or 99% sequence identity to SEQ ID NO: 60.

In some embodiments, the RNA element can comprise an engineered SmOPT variant sequence and an engineered U7 hairpin variant sequence having a polynucleotide sequence (expressed as RNA) of SEQ ID NO: 61. In some embodiments, the RNA element can comprise an engineered SmOPT variant sequence and an engineered U7 hairpin variant sequence having a polynucleotide sequence (expressed as RNA) with at least about: 80%, 85%, 90%, 92%, 95%, 97%, or 99% sequence identity to SEQ ID NO: 61.

In some embodiments, the RNA element can comprise an engineered SmOPT variant sequence and an engineered U7 hairpin variant sequence having a polynucleotide sequence (expressed as RNA) of SEQ ID NO: 62. In some embodiments, the RNA element can comprise an engineered SmOPT variant sequence and an engineered U7 hairpin variant sequence having a polynucleotide sequence (expressed as RNA) with at least about: 80%, 85%, 90%, 92%, 95%, 97%, or 99% sequence identity to SEQ ID NO: 62.

Some examples of the disclosure provide an engineered RNA (e.g., engineered guide RNA, ASO) described here comprising an RNA element, such as, an engineered U7 hairpin variant sequence or a variant or alteration of a U7 hairpin sequence of SEQ ID NO: 3 or SEQ ID NO: 4. The phrase “engineered U7 hairpin variant” as used herein means a non-naturally occurring hairpin, a modified hairpin, or a variant hairpin sequence compared to a naturally occurring hairpin sequence or unmodified hairpin sequence, where the engineered U7 hairpin variant sequence comprises: a polynucleotide substitution or at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30); 30 or fewer polynucleotide substitutions (e.g., 29, 28, 27, 26, 25, 24, 23, 22, 21, 20, 19, 18, 17, 16, 15, 14, 13, 12, 11, 10, 9, 8, 7, 6, 5, 4, 3, 2, 1); or from 1-30 polynucleotide substitutions (e.g., 2-29, 3-28, 4-27, 5-26, 6-25, 7-24, 8-23, 9-22, 10-21, 11-20, 12-19, 13-18, 14-17, 15-16) as compared to a wild type, a naturally occurring, or an unmodified sequence, such as a mouse U7 hairpin sequence of CAGGUUUUCUGACUUCGGUCGGAAAACCCCU (SEQ ID NO: 3). In some embodiments, the engineered U7 hairpin variant sequence comprises: a polynucleotide substitution or at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31); 31 or fewer polynucleotide substitutions (e.g., 30, 29, 28, 27, 26, 25, 24, 23, 22, 21, 20, 19, 18, 17, 16, 15, 14, 13, 12, 11, 10, 9, 8, 7, 6, 5, 4, 3, 2, 1); or from 1-31 polynucleotide substitutions (e.g., 2-30, 3-29, 4-28, 5-27, 6-26, 7-25, 8-24, 9-23, 10-22, 11-21, 12-20, 13-19, 14-18, 15-17) as compared to a wild type, a naturally occurring, or an unmodified sequence, such as a human U7 hairpin sequence of UAGGCUUUCUGGCUUUUUACCGGAAAGCCCCU (SEQ ID NO: 4). In some aspects, “engineered U7 hairpin variant” as used herein can refer to an engineered U7 hairpin, including a variant of a murine U7 hairpin sequence (CAGGUUUUCUGACUUCGGUCGGAAAACCCCU (SEQ ID NO: 3) or a variant of a human U7 sequence (UAGGCUUUCUGGCUUUUUACCGGAAAGCCCCU (SEQ ID NO: 4), where the engineered U7 hairpin variant sequence has up to and including 96.8% sequence identity to SEQ ID NO: 3 or up to and including 96.9% sequence identity to SEQ ID NO: 4. In some embodiments, when the engineered RNA (e.g., engineered guide RNAs or antisense oligonucleotides) comprises an engineered U7 hairpin variant sequence, the engineered U7 hairpin variant sequence has up to and including 96.8% sequence identity to SEQ ID NO: 3. In some embodiments, when the engineered RNA (e.g., engineered guide RNAs or antisense oligonucleotides) comprises an engineered U7 hairpin variant sequence, the engineered U7 hairpin variant sequence has up to and including 96.9% sequence identity to SEQ ID NO: 4.

In some embodiments, an engineered RNA can be an engineered guide RNA configured for editing of a base of a nucleotide of a target RNA (such as an engineered guide RNA having an RNA element having a polynucleotide sequence of any one of SEQ ID NO: 49, SEQ ID NO: 50, SEQ ID NO: 51, SEQ ID NO: 60, SEQ ID NO: 61, or SEQ ID NO: 62). RNA editing can be accomplished using an engineered guide RNA having a targeting sequence that is configured to hybridize to, is capable of hybridizing to, or a targeting sequence with sufficient complementarity to, a target RNA to allow for hybridization at least in part. The engineered RNA (e.g., engineered guide RNAs or ASOs) described here can comprise RNA elements of the engineered SmOPT variant sequence and the engineered U7 hairpin variant sequence. In some embodiments, the engineered guide RNAs comprising the RNA elements of the engineered SmOPT variant sequence and the engineered U7 hairpin variant facilitate an increase in an amount of editing of a base of a nucleotide of the target RNA by an RNA editing entity, relative to an otherwise comparable guide RNA lacking the engineered SmOPT variant sequence, the engineered U7 hairpin variant sequence, or combinations thereof as determined by an in vitro assay, such as, but not limited to, RNA sequencing.

Some examples described here provide the disclosed engineered RNA (e.g., engineered guide RNA, antisense oligonucleotide) comprises a targeting sequence with sufficient complementarity to a target RNA, capable of hybridizing to a target RNA, or combinations thereof; and RNA elements comprising an engineered SmOPT variant sequence and an engineered U7 hairpin variant sequence (including an RNA element having a polynucleotide sequence of any one of SEQ ID NO: 49, SEQ ID NO: 50, SEQ ID NO: 51, SEQ ID NO: 60, SEQ ID NO: 61, or SEQ ID NO: 62), where when the engineered RNA (e.g., engineered guide RNA, ASO) comprises or is operably linked to an engineered SmOPT variant sequence, where the engineered SmOPT variant sequence has up to and including 90.9% (e.g., about: 9%, 18%, 27%, 36%, 45%, 55%, 64%, 73%, 82%) sequence identity to AAUUUGUSKAG (SEQ ID NO: 1) or AAUUUUUGGAG (SEQ ID NO: 2); and where when the engineered RNA comprises or is operably linked to an engineered U7 hairpin variant sequence, the engineered U7 hairpin variant sequence has up to and including 96.8% (e.g., about: 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 4⁸%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96.7%) sequence identity to CAGGUUUUCUGACUUCGGUCGGAAAACCCCU (SEQ ID NO: 3) or the engineered U7 hairpin variant sequence has up to and including 96.9% (e.g., about: 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96.8%) sequence identity to UAGGCUUUCUGGCUUUUUACCGGAAAGCCCCU (SEQ ID NO: 4). In other embodiments, the disclosed engineered RNA (e.g., engineered guide RNA, ASO) comprises: (a) a targeting sequence with sufficient complementarity to a target RNA, capable of hybridizing to a target RNA, or combinations thereof; (b) an engineered SmOPT variant sequence, and (c) an engineered U7 hairpin variant sequence, where when the engineered RNA (e.g., engineered guide RNA, ASO) comprises an engineered SmOPT variant sequence of (b), the engineered SmOPT variant sequence has at least about 9% (e.g., about: 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.9%) sequence identity to AAUUUGUSKAG (SEQ ID NO: 1) or AAUUUUUGGAG (SEQ ID NO: 2); and where when the engineered RNA (e.g., engineered guide RNA, ASO) comprises an engineered U7 hairpin variant sequence of (c), the engineered U7 hairpin variant sequence has at least about 3% (e.g., 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96.7%, 96.8%) sequence identity to CAGGUUUUCUGACUUCGGUCGGAAAACCCCU (SEQ ID NO: 3) or the engineered U7 hairpin variant sequence has at least about 3% (e.g., 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96.7%, 96.8%, 96.9%) sequence identity to UAGGCUUUCUGGCUUUUUACCGGAAAGCCCCU (SEQ ID NO: 4). In yet further embodiments, the disclosed engineered RNA (e.g., engineered guide RNA, ASO) comprises: (a) a targeting sequence with sufficient complementarity to a target RNA, capable of hybridizing to a target RNA, or combinations thereof; (b) an engineered SmOPT variant sequence, and (c) an engineered U7 hairpin variant sequence, where when the engineered RNA comprises an engineered SmOPT variant sequence of (b), the engineered SmOPT variant sequence has about 9%-90.9% (e.g., 10%-90.8%; 11%-90%; 12%-89%; 13%-88%; 14%-87%; 15%-86%; 16%-85%; 17%-84%; 18%-83%; 19%-82%; 20%-81%; 21%-80%; 22%-79%; 23%-78%; 24%-77%; 25%-76%; 26%-75%; 27%-74%; 28%-73%; 29%-72%; 30%-71%; 31%-70%; 32%-69%; 33%-68%; 34%-67%; 35%-66%; 36%-65%; 37%-64%; 38%-63%; 39%-62%; 40%-61%; 41%-60%; 42%-59%; 43%-58%; 44%-57%; 45%-56%; 46%-55%; 47%-54%; 48%-53%; 49%-52%; 50%-51%) sequence identity to AAUUUGUSKAG (SEQ ID NO: 1) or AAUUUUUGGAG (SEQ ID NO: 2); and where when the engineered RNA (e.g., engineered guide RNA, ASO) comprises an engineered U7 hairpin variant sequence of (c), the engineered U7 hairpin variant sequence has about 3%-96.8% (e.g., 4%-96.7%; 5%-96%; 6%-95%; 7%-94%; 8%-93%; 9%-92%; 10%-91%; 11%-90%; 12%-89%; 13%-88%; 14%-87%; 15%-86%; 16%-85%; 17%-84%; 18%-83%; 19%-82%; 20%-81%; 21%-80%; 22%-79%; 23%-78%; 24%-77%; 25%-76%; 26%-75%; 27%-74%; 28%-73%; 29%-72%; 30%-71%; 31%-70%; 32%-69%; 33%-68%; 34%-67%; 35%-66%; 36%-65%; 37%-64%; 38%-63%; 39%-62%; 40%-61%; 41%-60%; 42%-59%; 43%-58%; 44%-57%; 45%-56%; 46%-55%; 47%-54%; 48%-53%; 49%-52%; 50%-51%) sequence identity to CAGGUUUUCUGACUUCGGUCGGAAAACCCCU (SEQ ID NO: 3) or the engineered U7 hairpin variant sequence has from about 3%-96.9% (e.g., 4%-96.8%; 5%-96.7%; 6%-96%; 7%-95%; 8%-94%; 9%-93%; 10%-92%; 11%-91%; 12%-90%; 13%-89%; 14%-88%; 15%-87%; 16%-86%; 17%-85%; 18%-84%; 19%-83%; 20%-82%; 21%-81%; 22%-80%; 23%-79%; 24%-78%; 25%-77%; 26%-76%; 27%-75%; 28%-74%; 29%-73%; 30%-72%; 31%-71%; 32%-70%; 33%-69%; 34%-68%; 35%-67%; 36%-66%; 37%-65%; 38%-64%; 39%-63%; 40%-62%; 41%-61%; 42%-60%; 43%-59%; 44%-58%; 45%-57%; 46%-56%; 47%-55%; 48%-54%; 49%-53%; 50%-52%) sequence identity to UAGGCUUUCUGGCUUUUUACCGGAAAGCCCCU (SEQ ID NO: 4).

In some aspects, the disclosed engineered RNA (e.g., engineered guide RNA, ASO) comprises (a) a targeting sequence with sufficient complementarity to a target RNA, capable of hybridizing to a target RNA, or combinations thereof; (b) an engineered SmOPT variant sequence; and (c) an engineered U7 hairpin variant sequence, where when the engineered RNA comprises an engineered SmOPT variant sequence, the engineered SmOPT variant sequence comprises at least one polynucleotide substitution (e.g., 1, 2, 3, 4, 5, 6, 7, 8, 9, 10, 11); 11 or fewer polynucleotide substitutions (e.g., 10, 9, 8, 7, 6, 5, 4, 3, 2, 1); or from 1-11 polynucleotide substitutions (e.g., 2-10, 3-9, 4-8, 5-7) of N₁N₂N₃N₄N₅N₆N₇N₈N₉N₁₀N₁₁ as compared to SEQ ID NO: 1 or SEQ ID NO: 2; and where when the engineered RNA (e.g., engineered guide RNA, ASO) comprises an engineered U7 hairpin variant sequence, the engineered U7 hairpin variant sequence comprises at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32); 32 or 31 or fewer polynucleotide substitutions (e.g., 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1); or from 1-32 polynucleotide substitutions or 1-31 polynucleotide substitutions (e.g., 2-31, 3-30, 4-29, 5-28, 6-27, 7-26, 8-25, 9-24, 10-23, 11-22, 12-21, 13-20, 14-19, 15-18, 16-17) of N₁N₂N₃N₄N₅N₆N₇N₈N₉N₁₀N₁₁N₁₂N₁₃N₁₄N₁₅N₁₆N₁₇N₁₈N₁₉N₂₀N₂₁N₂₂N₂₃N₂₄N₂₅N₂₆N₂₇N₂₈N₂₉N₃₀N₃₁N₃₂ as compared to SEQ ID NO: 3 or SEQ ID NO: 4, where N₁-N₃₁ is independently A, U, G, or C, and n₃₂ is A, U, G, C, or absent. In some embodiments of the disclosure when the engineered RNA (e.g., engineered guide RNA, ASO) comprises an engineered SmOPT variant sequence, where the engineered SmOPT variant sequence is not SEQ ID NO: 1 or SEQ ID NO: 2, or when the engineered RNA comprises an engineered U7 hairpin variant sequence, where the engineered U7 hairpin variant sequence is not SEQ ID NO: 3 or where the engineered U7 hairpin variant sequence is not SEQ ID NO: 4.

Some embodiments provide the engineered RNA (e.g., engineered guide RNA, ASO) of the disclosure comprising (b) the engineered SmOPT variant sequence, the engineered SmOPT variant sequence comprises at least one polynucleotide substitution (e.g., 1, 2, 3, 4, 5, 6, 7, 8, 9, 10, 11); 11 or fewer polynucleotide substitutions (e.g., 10, 9, 8, 7, 6, 5, 4, 3, 2, 1); or from 1-11 polynucleotide substitutions (e.g., 2-10, 3-9, 4-8, 5-7) of AAUUUN₁UN₂N₃AG, wherein each of N₁, N₂, and N₃ are independently A, U, G, or C, with the proviso that: when N₁ of SEQ ID NO: 7 is G, then N₂ is A, U, or G; or N₃ is A, G, or C; or when N₁ of SEQ ID NO:7 is U, then at least one of N₂ and N₃ is A, U, or C; when N₂ of SEQ ID NO: 7 is C, then N₁ is A, U, or C; or N₁ is A, G, or C; or when N₂ of SEQ ID NO: 7 is G, then N₁ is A, G, or C; or N₁ is A, U, or C; when N₃ of SEQ ID NO: 7 is U, then N₁ is A, U, or C; or N₂ is A, U, or G; or when N₃ of SEQ ID NO: 3 A is G, then N₁ is A, G, or C, or N₂ is A, U, or C, or when the engineered RNA comprises the engineered SmOPT variant sequence of, the engineered SmOPT variant sequence is not SEQ ID NO: 1 or SEQ ID NO: 2. In some cases, the engineered SmOPT variant can comprise a G to U substitution at nucleotide 6 of SEQ ID NO: 1. In some cases, the engineered SmOPT variant can comprise a G to U substitution at nucleotide 6 of SEQ ID NO: 1. In some cases, the engineered SmOPT variant comprises an A to C substitution at nucleotide 1 of SEQ ID NO: 1. In some cases, the engineered SmOPT variant comprises a G to A substitution at nucleotide 8 of SEQ ID NO: 2. In some cases, the engineered SmOPT variant comprises a G to A, C, or U substitution at nucleotide 9 of SEQ ID NO: 2. In some cases, the engineered SmOPT variant comprises an A to C substitution at nucleotide 10 of SEQ ID NO: 2. In some cases, the engineered SmOPT variant comprises a G to A, C, or U substitution at nucleotide 11 of SEQ ID NO: 2. In some cases, the 5′ end of SEQ ID NO: 2 comprises a U or an C insertion. In some cases, the 3′ end of SEQ ID NO: 2 comprises a U or an A insertion. In some cases, nucleotides 3, 4, 5, 6 and 7 of SEQ ID NO: 2 each comprise a U.

In some examples, the engineered RNA (e.g., engineered guide RNA, ASO) described here comprises (c) engineered U7 hairpin variant sequence the engineered U7 hairpin variant sequence comprising: at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32); 32 or fewer polynucleotide substitutions (e.g., 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1); or from 1-32 polynucleotide substitutions (e.g., 2-31, 3-30, 4-29, 5-28, 6-27, 7-26, 8-25, 9-24, 10-23, 11-22, 12-21, 13-20, 14-19, 15-18, 16-17) of N₁AGGN₂UUUCUGN₃CUUN₄N₅N₆N₇CN₈GN₉AAAN₁₀CCCN₁₁N₁₂ (SEQ ID NO: 8), wherein each of N₁, N₂, N₃, N₄, N₅, N₆, N₇, N₈, N₉, N₁₀, N₁₁, and N₁₂ are independently A, U, G, or C, with the proviso that: where if N₁ of SEQ ID NO: 8 is C, then at least one of N₂, N₇, and N₁₁ is A, G, or C; or at least one of N₃ and N₉ is U, G, or C; or at least one of N₄ and N₁₀ is A, U, or G; or at least one of N₅, N₆, and N₈ is A, U, or C; and where N₁₂ is A, U, G, C, or absent (none); or if N₁ of SEQ ID NO: 8 is U, then at least one of N₂, N₈, and N₁₁ is A, U, or G; or at least one of N₃, N₉, and N₁₀ is A, U, or C; or at least one of N₄, N₅, N₆, and N₁₂ is A, G, or C; or N₇ is U, G, or C; where if N₂ of SEQ ID NO: 8 is U, then at least one of N₁, N₄, and N₁₀ is A, U, or G; or at least one of N₃ and N₉ is U, G, or C; or at least one of N₅, N₆, and N₈ is A, U, or C; or at least one of N₇ and N₁ is A, G, or C; and N₁₂ is A, U, G, C, or absent; or if N₂ of SEQ ID NO: 8 is C, then at least one of N₁, N₄, N₅, N₆, and N₁₂ is A, G, or C; or at least one of N₃, N₉, and N₁₀ is A, U, or C; or N₇ is U, G, or C; or at least one of N₈ and N₁₁ is A, U, or G; or where if N₃ of SEQ ID NO: 8 is A, then at least one of N₁, N₄, and N₁₀ is A, U, or G; or at least one of N₂, N₇, and N₁₁ is A, G, or C; or at least one of N₅, N₆, and N₈ is A, U, or C; or N₉ is U, G, or C; and N₁₂ is A, U, G, C, or absent; or if N₃ of SEQ ID NO: 8 is G, then at least one of N₁, N₄, N₅, N₆, and N₁₂ is A, G, or C; or at least one of N₂, N₈, and N₁₁ is A, U, or G; or N₇ is U, G, or C; or at least one of N₉ and N₁₀ is A, U, or C; or where if N₄ of SEQ ID NO: 8 is C, then at least one of N₁ and N₁₀ is A, U, or G; or at least one of N₂, N₇, and N₁₁ is A, G, or C; or at least one of N₃ and N₉ is U, G, or C; or at least one of N₅, N₆, and N₈ is A, U, or C; and N₁₂ is A, U, G, C, or absent; or if N₄ of SEQ ID NO: 8 is U, then at least one of N₁, N₅, N₆, and N₁₂ is A, G, or C; or at least one of N₂, N₈, and N₁₁ is A, U, or G; or at least one of N₃, N₉, and N₁₀ is A, U, or C; or N₇ is U, G, or C; or where if N₅ of SEQ ID NO: 8 is G, then at least one of N₁, N₄, and N₁₀ is A, U, or G; or at least one of N₂, N₇, and N₁₁ is A, G, or C; or at least one of N₃ and N₉ is U, G, or C; or at least one of N₆ and N₈ is A, U, or C; and N₁₂ is A, U, G, C, or absent; or if N₅ of SEQ ID NO: 8 is U, then at least one of N₁, N₄, N₆, and N₁₂ is A, G, or C; or at least one of N₂, N₈, and N₁₁ is A, U, or G; or at least one of N₃, N₉, and N₁₀ is A, U, or C; or N₇ is U, G, or C; or where if N₆ of SEQ ID NO: 8 is G, then at least one of N₁, N₄, and N₁₀ is A, U, or G; or at least one of N₂, N₇, and N₁₁ is A, G, or C; or at least one of N₃ and N₉ is U, G, or C; or at least one of N₅ and N₈ is A, U, or C; and N₁₂ is A, U, G, C, or absent; or if N₆ of SEQ ID NO: 8 is U, then at least one of N₁, N₄, N₅, and N₁₂ is A, G, or C; or at least one of N₂, N₈, and N₁₁ is A, U, or G; or at least one of N₃, N₉, and N₁₀ is A, U, or C; or N₇ is U, G, or C; or where if N₇ of SEQ ID NO: 8 is U, then at least one of N₁, N₄, and N₁₀ is A, U, or G; or at least one of N₂ and N₁₁ is A, G, or C; or at least one of N₃ and N₉ is U, G, or C; or at least one of N₅, N₆, and N₈ is A, U, or C; and N₁₂ is A, U, G, C, or absent; or if N₇ of SEQ ID NO: 8 is A, then at least one of N₁, N₄, N₅, N₆, and N₁₂ is A, G, or C; or at least one of N₂, N₈, and N₁₁ is A, U, or G; or at least one of N₃, N₉, and N₁₀; or where if N₈ of SEQ ID NO: 8 is G, then at least one of N₁, N₄, and N₁₀ is A, U, or G; or at least one of N₂, N₇, and N₁₁ is A, G, or C; or at least one of N₃ and N₉ is U, G, or C; or at least one of N₅ and N₆ is A, U, or C; and N₁₂ is A, U, G, C, or absent; or if N₈ of SEQ ID NO: 8 is C, then at least one of N₁, N₄, N₅, N₆, and N₁₂ is A, G, or C; or at least one of N₂ and N₁₁ is A, U, or G; or at least one of N₃, N₉, and N₁₀ is A, U, or C; or N₇ is U, G, or C; or where if N₉ of SEQ ID NO: 8 is A, then at least one of N₁, N₄, and N₁₀ is A, U, or G; or at least one of N₂, N₇, and N₁₁ is A, G, or C; or N₃ is U, G, or C; or at least one of N₅, N₆, and N₈ is A, U, or C; and N₁₂ is A, U, G, C, or absent; or if N₉ of SEQ ID NO: 8 is G, then at least one of N₁, N₄, N₅, N₆, and N₁₂ is A, G, or C; or at least one of N₂, N₈, and N₁₁ is A, U, or G; or N₇ is U, G, or C; or at least one of N₃ and N₁₀ is A, U, or C; or where if N₁₀ of SEQ ID NO: 8 is C, then at least one of N₁ and N₄ is A, U, or G; or at least one of N₂, N₇, and N₁₁ is A, G, or C; or at least one of N₃ and N₉ is U, G, or C; or at least one of N₅, N₆, and N₈ is A, U, or C; and where N₁₂ is A, U, G, C, or absent; or if N₁₀ of SEQ ID NO: 8 is G, then at least one of N₁, N₄, N₅, N₆, and N₁₂ is A, G, or C; or at least one of N₂, N₈, and N₁₁ is A, U, or G; or at least one of N₃ and N₉ is A, U, or C; or N₇ is U, G, or C; or where if N₁₁ of SEQ ID NO: 8 is U, then at least one of N₁, N₄, and N₁₀ is A, U, or G; or at least one of N₂ and N₇ is A, G, or C; or at least one of N₃ and N₉ is U, G, or C; or at least one of N₅, N₆, and N₈ is A, U, or C; and N₁₂ is A, U, G, C, or absent; or if N₁₁ of SEQ ID NO: 8 is C, then at least one of N₁, N₄, N₅, N₆, and N₁₂ is A, G, or C; or at least one of N₂ and N₈ is A, U, or G; or at least one of N₃, N₉, and N₁₀ is A, U, or C; or N₇ is U, G, or C; or where if N₁₂ of SEQ ID NO: 8 is absent, then at least one of N₁, N₄, and N₁₀ is A, U, or G; or at least one of N₂, N₇, and N₁₁ is A, G, or C; or at least one of N₃ and N₉ is U, G, or C; or at least one of N₅, N₆, and N₈ is A, U, or C; or if N₁₂ of SEQ ID NO: 8 is U, then at least one of N₁, N₄, N₅, and N₆; or at least one of N₂, N₈, and N₁₁ is A, U, or G; or at least one of N₃, N₉, and N₁₀ is A, U, or C; or N₇ is U, G, or C.

In some embodiments, when the engineered RNA (e.g., engineered guide RNA, ASO) comprises an engineered SmOPT variant sequence of (b), the engineered SmOPT variant sequence comprising an engineered Sm or Sm-like protein binding domain sequence comprises at least one polynucleotide substitution as compared to the wild-type or the unmodified Sm or Sm-like protein binding domain polynucleotide sequence of SEQ ID NO: 1 or SmOPT sequence of SEQ ID NO: 2. One type of spliceosomal protein includes Sm proteins that are commonly found in small nuclear ribonucleoproteins (snRNPs) and are found in the nucleus of eukaryotic cells. snRNPs are associated with several different functions including pre-mRNA splicing, rRNA processing; histone mRNA 3′ end processing, telomere replication, tRNA maturation, and the like. Sm and Sm-like proteins are members of family of polypeptides in eukaryotes that not only are small proteins (˜8-28 kDa), but also share a common domain known as the Sin domain. The sequence or spliceosomal sequence of the engineered RNA disclosed here can in some examples, comprise an Sm or Sm-like protein binding domain from a spliceosomal small nuclear RNA (snRNA) or a non-spliceosomal snRNA; and a hairpin from a spliceosomal snRNA or a non-spliceosomal snRNA. In some embodiments, SEQ ID NO: 1 can comprise a U1Sm sequence (AAUUUGUGGAG SEQ ID NO: 20) or a U7Sm sequence (AAUUUGUCUAG SEQ ID NO: 21).

In some embodiments, the engineered RNA (e.g., engineered guide RNA, ASO) described here, when comprising an RNA element of an engineered SmOPT variant sequence of (b) (such as an RNA element having a polynucleotide sequence of any one of SEQ ID NO: 49, SEQ ID NO: 50, SEQ ID NO: 51, SEQ ID NO: 60, SEQ ID NO: 61, or SEQ ID NO: 62), the engineered SmOPT variant sequence comprises at least one polynucleotide substitution (e.g., 1, 2, 3, 4, 5, 6, 7, 8, 9, 10, 11); 11 or fewer polynucleotide substitutions (e.g., 10, 9, 8, 7, 6, 5, 4, 3, 2, 1); or from 1-11 polynucleotide substitutions (e.g., 2-10, 3-9, 4-8, 5-7), where a polynucleotide substitution of the sequence of (b) as used here means one or more nucleoside or base changes of: AAUUUGUSKAG (SEQ ID NO: 1; Sm binding domain sequence), AAUUUUUGGAG (SEQ ID NO: 2; SmOPT sequence), or a wild-type or unmodified Sm or Sm-like protein binding domain sequence, and where the engineered guide RNA comprising the described engineered SmOPT variant sequence, the engineered SmOPT variant sequence, having at least one polynucleotide substitution, maintains sufficient functionality and activity to facilitate an increase in RNA levels or increase in an amount of editing of a base of a nucleotide of the target RNA by an RNA editing entity, relative to an otherwise comparable RNA lacking the engineered SmOPT variant sequence or the engineered Sm or Sm-like protein binding domain sequence as determined by an in vitro assay, such as but not limited to, RNA sequencing.

Some embodiments of the disclosure provide for an engineered RNA (e.g., engineered guide RNA, ASO) described here, where when the engineered RNA (e.g., engineered guide RNA, ASO) comprises an RNA element of engineered U7 hairpin variant sequence (such as an RNA element having a polynucleotide sequence of any one of SEQ ID NO: 49, SEQ ID NO: 50, SEQ ID NO: 51, SEQ ID NO: 60, SEQ ID NO: 61, or SEQ ID NO: 62), the engineered U7 hairpin variant sequence comprises at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30); 32 or fewer polynucleotide substitutions (e.g., 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1); or from 1-30 polynucleotide substitutions (e.g., 2-29, 3-28, 4-27, 5-26, 6-25, 7-24, 8-23, 9-22, 10-21, 11-20, 12-19, 13-18, 14-17, 15-16), where a polynucleotide substitution of the hairpin of (c) as used here means one or more nucleoside or base changes of: for example, CAGGUUUUCUGACUUCGGUCGGAAAACCCCU (SEQ ID NO: 3) or the wild-type or unmodified mouse U7 hairpin; or where when the engineered RNA (e.g., engineered guide RNA, ASO) comprises an RNA element of an engineered U7 hairpin variant sequence, the engineered U7 hairpin variant sequence comprises at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31); 31 or fewer polynucleotide substitutions (e.g., 30, 29, 28, 27, 26, 25, 24, 23, 22, 21, 20, 19, 18, 17, 16, 15, 14, 13, 12, 11, 10, 9, 8, 7, 6, 5, 4, 3, 2, 1); or from 1-31 polynucleotide substitutions (e.g., 2-29, 3-28, 4-27, 5-26, 6-25, 7-24, 8-23, 9-22, 10-21, 11-20, 12-19, 13-18, 14-17, 15-16), where a polynucleotide substitution of the hairpin of (c) as used here means one or more nucleoside or base changes of: UAGGCUUUCUGGCUUUUUACCGGAAAGCCCCU (SEQ ID NO: 4) or the wild-type or unmodified human U7 hairpin sequence. In some embodiments, the engineered guide RNA comprising the RNA element of the described engineered U7 hairpin variant sequence having at least one polynucleotide substitution maintains sufficient functionality and activity to facilitate an increase in RNA levels or increase in an amount of editing of a base of a nucleotide of the target RNA by an RNA editing entity, relative to an otherwise comparable guide RNA lacking engineered U7 hairpin variant sequence the engineered U7 hairpin variant sequence as determined by an in vitro assay, such as but not limited to RNA sequencing.

In some embodiments, the engineered U7 hairpin variant sequence comprises a G insertion at nucleotide 3 of SEQ ID NO: 3. In some cases, the engineered U7 hairpin variant sequence comprises an A to U substitution at nucleotide 2 of SEQ ID NO: 3. In some cases, the engineered U7 hairpin variant sequence comprises a U to G, C, or A substitution at nucleotide 5 of SEQ ID NO: 3. In some cases, the engineered U7 hairpin variant sequence comprises a U to C substitution at nucleotide 6 of SEQ ID NO: 3. In some cases, the engineered U7 hairpin variant sequence comprises a U to G substitution at nucleotide 8 of SEQ ID NO: 3. In some cases, the engineered U7 hairpin variant sequence comprises a U to C or A substitution at nucleotide 10 of SEQ ID NO: 3. In some cases, the engineered U7 hairpin variant sequence comprises a G to C substitution at nucleotide 11 of SEQ ID NO: 3. In some cases, the engineered U7 hairpin variant sequence comprises an A to C substitution at nucleotide 12 of SEQ ID NO: 3.

In some examples, the engineered guide RNA of the description provides (a) a targeting sequence; (b) an engineered Sm or Sm-like protein binding domain sequence, e.g., engineered SmOPT variant sequence; and (c) an engineered U7 hairpin variant sequence, and the engineered SmOpt variant sequence, the engineered U7 hairpin variant, facilitate an increase in RNA levels or an increase in an amount of editing of a base of a nucleotide of the target RNA by an RNA editing entity, relative to an otherwise comparable guide RNA lacking: the engineered SmOPT variant sequence, the engineered U7 hairpin variant sequence, or both, as determined by an in vitro assay, such as but not limited to, RNA sequencing.

In some examples, the engineered RNA (e.g., engineered guide RNA, ASO) comprising the engineered Sm or Sm-like protein binding domain. In some embodiments, the engineered RNA (e.g., engineered guide RNA, ASO) comprising (b) the engineered Sm or Sm-like protein binding domain can have at least one polynucleotide substitution as compared to an unmodified Sm or Sm-like protein binding domain polynucleotide sequence of SEQ ID NO: 1 or SEQ ID NO: 2. Some embodiments can provide for the engineered RNA (e.g., engineered guide RNA, ASO) comprising the engineered Sm or Sm-like protein binding domain having two, three, or four polynucleotide substitutions as compared to the unmodified Sm or Sm-like protein binding domain polynucleotide sequence of SEQ ID NO: 1 or SEQ ID NO: 2.

Some embodiments can provide for an engineered RNA (e.g., engineered guide RNA, ASO) comprising the engineered U7 hairpin variant sequence. In some examples, the engineered U7 hairpin variant sequence can comprise at least one polynucleotide substitution as compared to an unmodified hairpin polynucleotide sequence of SEQ ID NO: 3 or SEQ ID NO: 4. Some embodiments can provide for the engineered U7 hairpin variant sequence having 2-15 polynucleotide substitutions as compared to the unmodified hairpin polynucleotide sequence of SEQ ID NO: 3 or SEQ ID NO: 4. In yet some examples, the engineered U7 hairpin variant sequence can have two, three, five, or ten polynucleotide substitutions as compared to the unmodified hairpin polynucleotide sequence of SEQ ID NO: 3 or SEQ ID NO: 4.

In some embodiments, the engineered RNA of the disclosure comprises, from 5′ to 3′ (a) a targeting sequence (b) the engineered Sm or Sm-like protein binding domain variant of the disclosure; and (c) the engineered U7 hairpin variant sequence of the disclosure. In some embodiments, the engineered RNA of the disclosure can comprise a wildtype Sm or Sm-like protein binding domain and the engineered U7 hairpin variant sequence of the disclosure. In some embodiments, the engineered RNA of the disclosure can comprise the engineered Sm or Sm-like protein binding domain variant of the disclosure and a wildtype U7 hairpin sequence.

In some embodiments of the disclosure, the engineered RNAs comprising the targeting sequence and RNA elements of the engineered SmOPT variant sequence (e.g., modified or variant Sm or Sm-like protein binding domain) and the engineered U7 hairpin variant sequence (e.g., modified or variant U7 hairpin, modified or variant mouse U7 snRNA hairpin, modified or variant human U7 snRNA hairpin) can be located 3′ of the at least one mismatch, which occurs when the targeting sequence and target RNA are hybridized.

### Engineered RNAs

The engineered RNAs (e.g., engineered guide RNAs, antisense oligonucleotides (ASOs)) described here comprise a targeting sequence that can be operably linked to RNA elements (e.g., engineered SmOPT variant sequence, engineered U7 hairpin variant sequence and combinations thereof such as an element having a polynucleotide sequence of any one of SEQ ID NO: 49, SEQ ID NO: 50, SEQ ID NO: 51, SEQ ID NO: 60, SEQ ID NO: 61, or SEQ ID NO: 62). In some embodiments, engineered RNAs described here comprise a targeting sequence that allows the engineered RNA to hybridize to a region of a target RNA or target RNA molecule. In some embodiments, the engineered RNAs (e.g., engineered guide RNAs, antisense oligonucleotides) described here comprise a targeting sequence with sufficient complementary to a target RNA for hybridization of the engineered RNA and target RNA. Various embodiments comprise engineered RNAs (e.g., engineered guide RNAs, antisense oligonucleotides) comprising a targeting sequence operably linked to an RNA element, such as an engineered SmOPT variant sequence. In other embodiments, the engineered RNAs (e.g., engineered RNAs, antisense oligonucleotides) of the disclosure comprise a targeting sequence operably linked to an RNA element, such as an engineered U7 hairpin variant sequence. Some embodiments of the disclosure provide engineered RNAs (e.g., engineered guide RNAs, antisense oligonucleotide) comprising a targeting sequence operably linked to an engineered SmOPT variant sequence and an engineered U7 hairpin variant sequence.

### Targeting Sequence

Engineered RNAs disclosed herein can be engineered or designed in any way suitable for RNA editing or altering RNA interactions, processing, or expression. In some examples, an engineered RNA generally comprises at least a targeting sequence that is capable of hybridizing to or, in some embodiments, a targeting sequence with target complementarity to a region of a target RNA or target RNA molecule, used interchangeably here. A targeting sequence can also be referred to as a “targeting domain” or a “targeting region” and used interchangeably here.

In some cases, a targeting sequence of an engineered RNA described here allows the engineered RNA to target an RNA sequence through base pairing, such as Watson Crick base pairing. In some examples, the targeting sequence can be located at either the N-terminus or C-terminus of the engineered RNA. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered RNA (e.g., engineered guide RNA, antisense oligonucleotide) of the disclosure comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered RNA described here comprises a targeting sequence that can be about 100 nucleotides in length.

In some cases, a targeting domain comprises 95%, 96%, 97%, 98%, 99%, or 100% sequence complementarity to a target RNA, of sufficient complementarity to a target RNA to hybridize. In some cases, a targeting sequence comprises less than 100% complementarity to a target RNA sequence and can hybridize in part to the target RNA. For example, a targeting sequence and a region of a target RNA that can be bound by the targeting sequence can have a single base mismatch.

Some embodiments of the disclosure provide an engineered RNA (e.g., engineered guide RNA, antisense oligonucleotide) described here comprising a targeting sequence with complementarity or sufficient complementarity to a target RNA thereby providing partial hybridization or complete hybridization of the targeting sequence and the target RNA that form guide-target RNA scaffold. A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more features selected from a bulge, mismatch, internal loop, hairpin, or wobble base pair. Various aspects of the disclosure provide targeting sequences having complete complementarity to a target RNA. In some examples, the engineered RNAs (e.g., engineered guide RNAs, antisense oligonucleotides) of the disclosure comprise a targeting sequence that is substantially complementary to a target RNA. Useful targeting sequences of the disclosure can have sufficient complementarity to a target RNA.

“Sufficient complementarity” as used here, can mean at least 5 nucleotides (e.g., 6, 7, 8, 9, 10, 11, 12, 13, 14, 15, 16, 17, 18, 19, 20, 30, 40, 50, 60, 70, 80, 90, 100, 200, 300, 400, 500, 600); or can mean 600 nucleotides or fewer (e.g., 550, 450, 350, 250, 150, 105, 95, 85, 75, 65, 55, 45, 35, 25, 15, 5) of the engineered RNA are complementary to or has base pairing to a target RNA; can mean 5-600 nucleotides (e.g., 10-550, 15-450, 20-350, 25-250, 30-150, 35-105, 40-95, 45-85, 50-75, 55-65) of the engineered RNA that are complementary to or has base pairing to a target RNA; can mean at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) of the engineered RNA nucleotides are complementary to the target RNA; can mean 100% or less (e.g., 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%); or can mean 70%-100% (e.g., 71%-99%, 72%-98%, 73%-97%, 74%-96%, 75%-95%, 76%-94%, 77%-93%, 78%-92%, 79%-91%, 80%-90%, 81%-89%, 82%-88%, 83%-87%, 84%-86) of the engineered RNA nucleotides are complementary to the target RNA.

In some examples, the described engineered RNA (e.g., engineered guide RNA, ASO) of the disclosure can comprise a targeting sequence that has target complementarity to a splice signal proximal to an exon within the target RNA.

Some embodiments can provide for the described engineered RNA of the disclosure, wherein the targeting sequence has (a) target complementarity to a branch point upstream of an exon within the target RNA; or (b) target complementarity to a donor splice site downstream of an exon within the target RNA. The engineered RNAs (e.g., engineered guide RNA, ASO) of the disclosure, in some embodiments, can comprise a targeting sequence having target complementarity to (a) a 3′ or 5′ untranslated region (UTR) of the target RNA; (b) a translation initiation site; (c) an intronic region of the target RNA; or (d) an exonic region of the target RNA.

In some embodiments, a double stranded RNA (dsRNA) substrate is formed upon hybridization of an engineered guide RNA of the present disclosure to a target RNA. The resulting dsRNA substrate is also referred to herein as a “guide-target RNA scaffold.” Described herein are “structural features” that can be present in a guide-target RNA scaffold of the present disclosure. Examples of structural features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. The term “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited. In some embodiments, structural features are not formed from latent structure and are, instead, pre-formed structures (e.g., a GluR2 recruitment hairpin or a hairpin from U7 snRNA).

In some embodiments, upon hybridization of the described engineered RNAs (e.g., engineered guide RNA, antisense oligonucleotide) and the described target RNAs, or at least a portion thereof such as the targeting sequence described here, can have one or more structural features (e.g., 2, 3, 4, 5, 6, 7, 8, 9, 10, 11, 12, 13, 14, 15, 16, 17, 18, 19, 20, 21, 22, 23, 24, 25); 50 or fewer structural features (e.g., 49, 48, 47, 46, 45, 40, 35, 30, 25, 20, 15, 10, 5, 1); or from 1-50 structural features (e.g., 2-49; 3-48; 4-47; 5-46; 6-45; 7-44; 8-43; 9-42; 10-41; 11-40; 12-39; 13-38; 14-37; 15-36; 16-35; 17-34; 18-33; 19-32; 20-31; 21-30; 22-29; 23-28; 24-27; 25-26; 1-5; 10-15; 15-20; 20-25; 25-30; 30-35; 35-40; 40-45; 45-50; 5-20; 1-3; 4-5; 2-10; 20-40; 10-40; 20-50; 30-50; 4-7; 8-10), where the one or more structural features is selected from the group consisting of: a bulge, a mismatch, an internal loop, a hairpin, a wobble base pair, and any of combinations thereof.

In some embodiments, the targeting sequence described here can be formed or configured to have at least one mismatched nucleotide (e.g., 2, 3, 4, 5) when hybridized to a target RNA. In some examples, a structural feature of the guide-target RNA scaffold formed upon hybridization of the engineered RNA and the target RNA of the disclosure, where the structural feature can comprise a wobble base pair, where the wobble base pair refers to two bases that weakly pair. For example, a wobble base pair of the present disclosure may refer to a G paired with a U.

Some examples of the disclosure provide the described engineered RNA (e.g., engineered guide RNA, antisense oligonucleotide) comprising a targeting sequence that where, upon hybridization of the targeting sequence to the target RNA, at least one mismatch forms, and the at least one mismatch comprises at least one adenosine-guanosine (A-G) mismatch, at least one adenosine-adenosine (A-A) mismatch, or at least one adenosine-cytidine (A-C), and where the adenosine (A) in the mismatch can be present in the target RNA. In examples of the disclosure, the engineered RNA described can have at least one mismatch comprising an A-C mismatch, where the adenosine in the mismatch can be present in the target RNA. Some embodiments provide for at least one mismatch that can be located from about 1 base to about 200 bases from either end of the targeting sequence.

Some aspects of the disclosure provide an engineered RNA comprising a targeting sequence capable of hybridizing entirely or in part, under, for example, stringent, moderate, or weak hybridization conditions, to a target RNA, thereby forming a guide-target RNA scaffold. In some examples, the guide-target RNA scaffold comprises at least one structural feature. “Stringent hybridization conditions” refers to, for example, an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 g/ml denatured, sheared salmon sperm DNA, followed by washing in 0.1×SSC at about 65° C. In some embodiments, engineered RNAs of the disclosure can hybridize to the target RNAs of the disclosure at lower stringency hybridization conditions. Changes in the stringency of hybridization can be accomplished through the manipulation of formamide concentration (lower percentages of formamide result in lowered stringency), salt conditions, or temperature. For example, lower stringency conditions include an overnight incubation at 37° C. in a solution comprising, for example, 6×SSPE (20×SSPE=3 M NaCl; 0.2 M NaH₂PO₄; 0.02 M EDTA, pH 7.4), 0.5% SDS, 30% formamide, 100 μg/ml salmon sperm blocking DNA, followed by washes at 50° C. with 1×SSPE, 0.1% SDS. In addition, to achieve even lower stringency, washes performed following stringent hybridization can be performed at higher salt concentrations (e.g., 5×SSC).

### Antisense Oligonucleotide Engineered RNAs

In some embodiments, the engineered RNAs of the disclosure can be antisense oligonucleotides (also referred to as ASOs) comprising short, chemically modified or synthesized single-stranded nucleotides. Some examples are directed to an engineered RNA that can be an antisense oligonucleotide substantially complementary to a target RNA. ASOs are chemically modified or synthesized DNA or RNA that can be substantially or fully complementary to a target sequence and designed or configured to inhibit, cover, mask, or block a target sequence. ASOs can be chemically modified to avoid degradation in view of their short length. ASOs can be DNA or RNA, but the DNA does not encode for the RNA. In some embodiments of the disclosure, the antisense oligonucleotides that are delivered can be RNA itself or DNA encoding for the RNA. Described herein are engineered RNA antisense oligonucleotides modified or altered from RNA found in nature that can modulate or alter RNA interactions, processing, expression, or combinations thereof. In some instances, an ASO designed or configured to inhibit, cover, mask, or block a target sequence of a target RNA promotes exon skipping of an exon in the target sequence. Methods have been used to induce exon skipping of a protein coding transcript. In many cases, a number of proteins such as alpha-synuclein and DMD can be expressed as different splice variants, some of which can be implicated in disease. It is thought that by promoting exon skipping events, exons containing a mutation implicated in a disease can be bypassed, or a codon reading frame can be restored, thereby facilitating the translation of variants that are sufficient to correct a disease or disorder, or alleviate symptoms of a disease or disorder.

Some examples described here provide antisense oligonucleotides of the disclosure comprising a single strand of 5 or more nucleotides (e.g., 6, 7, 8, 9, 10, 11, 12, 13, 14, 15, 16, 17, 18, 19, 20, 25, 30, 35, 40, 45, 50); 50 or fewer nucleotides (e.g., 49, 48, 47, 46, 45, 44, 43, 42, 41, 40, 39, 38, 37, 36, 35, 34, 33, 32, 31, 30, 25, 20, 15, 10, 5); or 5-50 nucleotides (e.g., 6-49, 7-48, 8-47, 9-46, 10-45, 11-44, 12-43, 13-42, 14-41, 15-40, 16-39, 17-38, 18-37, 19-36, 20-35, 21-34, 22-33, 23-32, 24-31, 25-30), which inhibit or block gene expression by hybridizing to a target RNA. In some embodiments, an ASO is from about 10 to about 200 nucleotides in length (for example, from about 10 to about 180 nucleotides, from about 15 to about 100 nucleotides, from about 15 to about 60 nucleotides, from about 20 to about 50 nucleotides, or from about 20 to about 40 nucleotides in length).

In some embodiments, an antisense oligonucleotide, described herein can comprise modifications. A modification can be a substitution, insertion, deletion, chemical modification, physical modification, stabilization, purification, or any combination thereof. In some cases, a modification can be a chemical modification. Antisense oligonucleotides, for example, can comprise chemical modifications to avoid degradation in view of their short length. Suitable chemical modifications comprise any one of: 5′ adenylate, 5′ guanosine-triphosphate cap, 5′ N₇-Methylguanosine-triphosphate cap, 5′ triphosphate cap, 3′ phosphate, 3′ thiophosphate, 5′ phosphate, 5′ thiophosphate, Cis-Syn thymidine dimer, trimers, C₁₂ spacer, C₃ spacer, C₆ spacer, dSpacer, PC spacer, rSpacer, Spacer 18, Spacer 9, 3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C₂, psoralen C₆, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, sugar modified analogs, wobble/universal bases, fluorescent dye label, 2′ fluoro RNA, 2′ O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3-phosphorothioate or any combinations thereof. In some embodiments, an antisense oligonucleotide does not comprise chemical modifications.

A chemical modification can be made at any location of the ASO, engineered guide RNA, or other RNA payload. In some cases, a modification may be located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. More than one modification can be made to the ASO, engineered guide RNA, or other RNA payload. In some cases, a modification can be permanent. In other cases, a modification can be transient. In some cases, multiple modifications may be made to the engineered RNA (ASO, engineered guide RNA, or other RNA payload). The modification to the engineered RNA can alter physio-chemical properties of a nucleotide, such as their conformation, polarity, hydrophobicity, chemical reactivity, base-pairing interactions, or any combination thereof.

A chemical modification can also be a phosphorothioate substitution. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some embodiments, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered RNA (ASO, engineered guide RNA, or other RNA payload) comprises at least one chemically modified base. In some instances, the engineered RNA comprises 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, the at least one chemical modification of the engineered RNA comprises a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered RNA include any modification contained herein, while some exemplary modifications are recited in Table 1.

TABLE 1
Exemplary Chemical Modification

| Modification of | |
|---|---|
| Engineered RNA | Examples |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or | Nucleic acid analog (examples of nucleotide analogs can be found |
| replacement of a naturally | in PCT/US2015/025175, PCT/US2014/050423, |
| occurring nucleobase | PCT/US2016/067353, PCT/US2018/041503, PCT/US18/041509, |
| | PCT/US2004/011786, or PCT/US2004/011833, all of which are |
| | expressly incorporated by reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

### Modification of Phosphate Backbone

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” may be meant to refer to a saturated hydrocarbon group which may be straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” may refer to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” may refer to an aliphatic group containing at least one double bond. As used herein, “alkynyl” may refer to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” may refer to an alkyl moiety in which an alkyl hydrogen atom may be replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” may refer to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” may refer to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” may refer to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way may be a stereogenic center. The stereogenic phosphorous atom can possess either the “R” configuration (herein Rp) or the “S” configuration (herein Sp). In some cases, the ASO can comprise stereopure nucleotides comprising S conformation of phosphorothioate or R conformation of phosphorothioate. In some embodiments, the chiral phosphate product may be present in a diastereomeric excess of 50%, 60%, 70%, 80%, 90%, or more. In some embodiments, the chiral phosphate product may be present in a diastereomeric excess of 95%. In some embodiments, the chiral phosphate product may be present in a diastereomeric excess of 96%. In some embodiments, the chiral phosphate product may be present in a diastereomeric excess of 97%. In some embodiments, the chiral phosphate product may be present in a diastereomeric excess of 98%. In some embodiments, the chiral phosphate product may be present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithionate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) may be attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetyl and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It may be also understood in a nucleotide substitute that both the sugar and the phosphate moieties of the nucleotide can be replaced, by for example an amide type linkage (aminoethylglycine) (PNA). It may be also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the engineered RNA described herein (ASO, engineered guide RNA, or other RNA payload) comprises at least one chemically modified phosphate backbone. Exemplary chemically modification of the phosphate group or backbone can include replacing one or more of the oxygens with a different substituent. Furthermore, the modified nucleotide present in the engineered RNA can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group may be achiral. However, replacement of one of the non-bridging oxygens with one of the above atoms or groups of atoms can render the phosphorous atom chiral; that may be to say that a phosphorous atom in a phosphate group modified in this way may be a stereogenic center. The stereogenic phosphorous atom can possess either the “R” configuration (herein Rp) or the “S” configuration (herein Sp). In such case, the chemically modified ASO can be stereopure (e.g. S or R confirmation). In some cases, the chemically modified engineered RNA comprises stereopure phosphate modification. For example, the chemically modified engineered RNA can comprise S conformation of phosphorothioate or R conformation of phosphorothioate.

Phosphorodithioates have both non-bridging oxygens replaced by sulfur. The phosphorus center in the phosphorodithioates may be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered RNA of the present disclosure (ASO, engineered guide RNA, or other RNA payload) can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetyl, oxime, methyleneimino, methylenemethylimino, methylenehydrazono, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, the chemical modification described herein comprises modification by replacement of a phosphate group. In some embodiments, the engineered RNA described herein (ASO, engineered guide RNA, or other RNA payload) comprises at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetyl, oxime, methyleneimino, methylenemethylimino, methylenehydrazono, methylenedimethylhydrazo and methyleneoxymethylimino.

### Modification of the Ribophosphate Backbone

In some embodiments, the chemical modification described herein comprises modifying ribophosphate backbone of the engineered RNA. In some embodiments, the engineered RNA described herein (ASO, engineered guide RNA, or other RNA payload) comprises at least one chemically modified ribophosphate backbone. Exemplary chemically modified ribophosphate backbone can include scaffolds that can mimic nucleic acids can also be constructed wherein the phosphate linker and ribose sugar may be replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein comprises modifying of sugar. In some embodiments, the engineered RNA described herein (ASO, engineered guide RNA, or other RNA payload) comprises at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)˜CH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a C₁₋₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein),NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the F position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that may be in the L form, e.g. L-nucleosides. In some aspects, the engineered RNA described herein (ASO, engineered guide RNA, or other RNA payload) includes the sugar group ribose, which may be a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose may be replaced by glycol units attached to phosphodiester bonds), threose nucleic acid. In some embodiments, the modifications to the sugar of the engineered RNA comprises modifying the engineered RNA to include locked nucleic acid (LNA), unlocked nucleic acid (UNA), or bridged nucleic acid (BNA).

### Modification of a Constituent of the Ribose Sugar

In some embodiments, the engineered RNA described herein (ASO, engineered guide RNA, or other RNA payload) comprises at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position may be unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the engineered RNA described herein ASO, engineered guide RNA, or other RNA payload) comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m may be from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′—(CH₂)—O-2′ (LNA); 4′—(CH₂)—S-2′; 4′—(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some embodiments, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the engineered RNA described herein (ASO, engineered guide RNA, or other RNA payload). The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein comprises modifying an uracil. In some embodiments, the engineered RNA described herein (ASO, engineered guide RNA, or other RNA payload) comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, l-taurinomethyl-4-thio-pseudouridine, 5-methyl-uridine, 1 methyl-pseudouridine, 5-methyl-2-thio-uridine, l-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroindene, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropyl pseudouridine, 5-(isopentenylaminomethyl) uridine, 5-(isopentenylaminomethyl])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein comprises modifying a cytosine. In some embodiments, the engineered RNA described herein (ASO, engineered guide RNA, or other RNA payload) comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying an adenine. In some embodiments, the engineered RNA described herein (ASO, engineered guide RNA, or other RNA payload) comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′—O-dimethyl-adenosine, N6-Methyl-2′-deoxyadenosine, N6, N6, 2′—O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the engineered RNA described herein (ASO, engineered guide RNA, or other RNA payload) comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2,7-dimethyl-guanosine, N2, N2,7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1, 2′—O-dimethyl-inosine, 6-O-phenyl-2′-deoxyinosine, 2′-O-ribosylguanosine, 1-thio-guanosine, 6-O-methylguanosine, O⁶-Methyl-2′-deoxyguanosine, 2′-F-ara-guanosine, and 2′-F-guanosine.

In some cases, the chemical modification of the engineered RNA can include introducing or substituting a nucleic acid analog or an unnatural nucleic acid into the engineered RNA. In some embodiments, nucleic acid analog can be any one of the chemically modified nucleic acid described herein. Exemplary nucleic acid analog can be found in PCT/US2021/034272, PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-azaadenosine, 2′-aracytidine, 2′-azauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopentenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylaminomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but may be not limited to, uracil-5-yl, hypoxanthin-9-yl (I), 2-aminoadenine-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base may be replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered RNA. In some embodiments, the engineered RNA can comprise a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group may be replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

### Engineered Guide RNAs

As disclosed herein, an engineered guide RNA of the present disclosure having RNA elements described herein (an SmOPT variant sequence, a U7 hairpin variant sequence, or both such as an element having a polynucleotide sequence of any one of SEQ ID NO: 49, SEQ ID NO: 50, SEQ ID NO: 51, SEQ ID NO: 60, SEQ ID NO: 61, or SEQ ID NO: 62) can be utilized for editing of a base of a nucleotide of a target RNA. The engineered guide RNAs described comprise a targeting sequence with sufficient complementarity to a target RNA operably linked to an RNA element described here, where the RNA element is an engineered SmOPT variant sequence, an engineered U7 hairpin variant sequence, or both. In some embodiments, the engineered guide RNAs described here can also comprise a promoter, a terminator, and additional elements disclosed here, for RNA editing.

In some examples of the disclosure, the engineered guide RNAs described here can have a length from about 80 nucleotides to about 600 nucleotides (e.g., 90-500, 100-400, 200-300); can have a length of at least about 80 nucleotides or greater (e.g., 85, 95, 150, 250, 350, 450, 550, 600, 650); or can have a length of about 600 nucleotides or fewer (e.g., 575, 525, 475, 425, 375, 325, 275, 225, 175, 125, 115, 110, 105, 95, 90, 85, 80, 75, 70).

### Circularized Guide RNA

In some instances, an engineered RNA can be circularized. A circularized engineered guide RNA can be produced from a precursor engineered polynucleotide. In some cases, a precursor engineered polynucleotide can be a precursor engineered linear polynucleotide. In some cases, a precursor engineered polynucleotide can be linear. For example, a precursor engineered polynucleotide can be a linear mRNA transcribed from a plasmid. In another example, a precursor engineered polynucleotide can be constructed to be a linear polynucleotide with domains such as a ribozyme domain and a ligation domain that allow for circularization in a cell. The linear polynucleotide with the ligation and ribozyme domains can be transfected into a cell where it can circularize via endogenous cellular enzymes. In some cases, a precursor engineered polynucleotide can be circular. In some cases, a precursor engineered polynucleotide can comprise DNA, RNA or both. In some cases, a precursor engineered polynucleotide can comprise a precursor engineered guide RNA. In some cases, a precursor engineered guide RNA can be used to produce an engineered guide RNA.

A circular or looped engineered guide polynucleotide, such as an engineered guide RNA can be formed directly or indirectly by forming a linkage (such as a covalent linkage) between more than one end of a RNA sequence, such as a 5′ end and a 3′ end. An RNA sequence can comprise an engineered guide RNA (such as a recruiting domain, targeting domain, or both). A linkage can be formed by employing an enzyme, such as a ligase. A suitable ligase (or synthetase) can include a ligase that forms a covalent bond. A covalent bond can include a carbon-oxygen bond, a carbon-sulfur bond, a carbon-nitrogen bond, a carbon-carbon bond, a phosphoric ester bond, or any combination thereof. A linkage can also be formed by employing a recombinase. An enzyme can be recruited to an RNA sequence to form a linkage. A circular or looped RNA can be formed by ligating more than one end of an RNA sequence using a linkage element. In some embodiments, a linkage can be formed by a ligation reaction. In some instances, a linkage can be formed by a homologous recombination reaction. A linkage element can employ click chemistry to form a circular or looped RNA. A linkage element can be an azide-based linkage. A circular or looped RNA can be formed by genetically encoding or chemically synthesizing the circular or looped RNA.

A circular or looped RNA can be formed by employing a self-cleaving entity, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor engineered RNA. In another example, a ribozyme, a tRNA, an aptamer, a catalytically active fragment of any of these, or any combination thereof can be added to a 3′ terminal end, a 5′ terminal end, or both of a precursor engineered RNA. A self-cleaving ribozyme can comprise, for example, an RNase P RNA a Hammerhead ribozyme (e.g. a Schistosoma mansoni ribozyme), a glmS ribozyme, an HDV-like ribozyme, an R2 element, a peptidyl transferase 23S rRNA, a GIR1 branching ribozyme, a leadzyme, a group II intron, a hairpin ribozyme, a VS ribozyme, a CPEB3 ribozyme, a CoTC ribozyme, or a group I intron. In some cases, the self-cleaving ribozyme can be a trans-acting ribozyme that joins one RNA end on which it is present to a separate RNA end. In some embodiments, an aptamer can be added to each end of the engineered guide RNA. A ligase can be contacted with the aptamers at each end of the engineered guide RNA to form a covalent linkage between the aptamers thereby forming a circular engineered guide RNA. In some cases, a self-cleaving element or an aptamer can be configured to facilitate self-circularization of an engineered polynucleotide or a pro-polynucleotide (e.g. from a precursor engineered polypeptide) after transcription in a cell. In some instances, circularization of a guide RNA can be shown by PCR. For example, primers can by developed that bind to the end of a guide RNA and are directed outward such that a product is only formed when guides are circularized.

In some cases, circularization can occur by back-slicing and ligation of an exon. For example, an RNA can be engineered from 5′ to 3′ to comprise a forward complementary sequence intron, an exon (which can comprise the guide sequence), followed by a reverse complementary sequence intron. Once transcribed, the complementary sequence introns can hybridize and form dsRNA. The internal exon containing the guide sequence can be removed by splicing and ligated by an endogenous ligase to form a circular guide. In one example, an engineered guide RNA can initiate circularization in a cell by autocatalytic reactions of encoded ribozymes. After cleavage by one or more ribozymes, the linear polynucleotide will undergo intracellular RNA ligation of the 5′ and the 3′ end of ligation sequences by an endogenous ligase to circularize the guide RNA.

A suitable self-cleaving molecule can include a ribozyme. For example, a ribozyme domain can create an autocatalytic RNA. A ribozyme can comprise an RNase P, an rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, an HDV ribozyme, a Twister ribozyme, a Twister sister ribozyme, a VS ribozyme, a Pistol ribozyme, a Hatchet ribozyme, a viroid, or any combination thereof. A ribozyme can include a P3 twister U2A ribozyme. A ribozyme can comprise 5′ GCCATCAGTCGCCGGTCCCAAGCCCGGATAAAATGGGAGGGGGCGGGAAACCGCCT 3′ (SEQ ID NO: 105). A ribozyme can comprise 5′ GCCAUCAGUCGCCGGUCCCAAGCCCGGAUAAAAUGGGAGGGGGCGGGAAACCGCCU 3′ (SEQ ID NO: 106). A ribozyme can comprise at least about: 70%, 75%, 80%, 85%, 90%, 95%, or 100% sequence homology to 5′ GCCATCAGTCGCCGGTCCCAAGCCCGGATAAAATGGGAGGGGGCGGGAAACCGCCT 3′ (SEQ ID NO: 105). A ribozyme can comprise at least about: 70%, 75%, 80%, 85%, 90%, 95%, or 100% sequence homology to 5′ GCCAUCAGUCGCCGGUCCCAAGCCCGGAUAAAAUGGGAGGGGGCGGGAAACCGCCU 3′ (SEQ ID NO: 106). A ribozyme can include a P1 Twister Ribozyme. A ribozyme can include 5′ AACACTGCCAATGCCGGTCCCAAGCCCGGATAAAAGTGGAGGGTACAGTCCACGC 3′ (SEQ ID NO: 107). A ribozyme can include 5′ AACACUGCCAAUGCCGGUCCCAAGCCCGGAUAAAAGUGGAGGGUACAGUCCACGC 3′ (SEQ ID NO: 108). A ribozyme can comprise at least about: 70%, 75%, 80%, 85%, 90%, 95%, or 100% sequence homology to 5′ AACACTGCCAATGCCGGTCCCAAGCCCGGATAAAAGTGGAGGGTACAGTCCACGC 3′ (SEQ ID NO: 107). A ribozyme can comprise at least about: 70%, 75%, 80%, 85%, 90%, 95%, or 100% sequence homology to 5′ AACACUGCCAAUGCCGGUCCCAAGCCCGGAUAAAAGUGGAGGGUACAGUCCACGC 3′ (SEQ ID NO: 108).

A ligation domain can facilitate a linkage, covalent or non-covalent, of a first nucleotide to a second nucleotide. In some embodiments, a ligation domain can recruit a ligating entity to facilitate a ligation reaction. In some cases, a ligation domain can recruit a recombining entity to facilitate a homologous recombination. In some instances, a first ligation domain can facilitate a linkage, covalent or non-covalent, to a second ligation domain. In some embodiments, a first ligation domain can facilitate the complementary pairing of a second ligation domain. In some cases, a ligation domain can comprise 5′ AACCATGCCGACTGATGGCAG 3′ (SEQ ID NO: 109). In some embodiments, a ligation domain can comprise 5′ GATGTCAGGTGCGGCTGACTACCGTC 3′ (SEQ ID NO: 110). In some cases, a ligation domain can comprise 5′ AACCAUGCCGACUGAUGGCAG 3′ (SEQ ID NO: 111). In some cases, a ligation domain can comprise 5′ GAUGUCAGGUGCGGCUGACUACCGUC 3′ (SEQ ID NO: 112). In some cases, a ligation domain can comprise at least about: 70%, 75%, 80%, 85%, 90%, 95%, or 100% sequence homology to 5′AACCATGCCGACTGATGGCAG 3′ (SEQ ID NO: 109). In some cases, a ligation domain can comprise at least about: 70%, 75%, 80%, 85%, 90%, 95%, or 100% sequence homology to 5′ GATGTCAGGTGCGGCTGACTACCGTC 3′ (SEQ ID NO: 110). In some cases, a ligation domain can comprise at least about: 70%, 75%, 80%, 85%, 90%, 95%, or 100% sequence homology to 5′ AACCAUGCCGACUGAUGGCAG 3′ (SEQ ID NO: 111). In some cases, a ligation domain can comprise at least about: 70%, 75%, 80%, 85%, 90%, 95%, or 100% sequence homology to 5′ GAUGUCAGGUGCGGCUGACUACCGUC 3′ (SEQ ID NO: 112).

### Engineered Guide RNAs Having a Recruiting Domain

In some examples, an engineered guide RNA can comprise a recruiting domain that is formed and present in the absence of hybridization of the engineered guide RNA to a target RNA, where the recruiting domain recruits an RNA editing entity (e.g., ADAR, APOBEC, or both). A “recruiting domain” can be referred to herein interchangeably as a “recruiting sequence” or a “recruiting region.” In some examples, an engineered guide RNA can be configured to facilitate editing of a base of a nucleotide of a polynucleotide of a region of a target RNA, modulation expression of a polypeptide encoded by the target RNA, or both. In some cases, an engineered guide RNA can be configured to facilitate an editing of a base of a nucleotide or polynucleotide of a region of an RNA by an RNA editing entity. In order to facilitate editing, an engineered guide RNA of the disclosure can be configured to recruit an RNA editing entity. Some embodiments provide for an RNA editing entity comprising an ADAR protein, where the ADAR protein can be selected from the group consisting of an ADAR1 (e.g., human or mouse), an ADAR2 (e.g., human or mouse), and any combination thereof. Various RNA editing entity recruiting domains can be utilized. In some examples, a recruiting domain comprises: Glutamate ionotropic receptor AMPA type subunit 2 (GluR2), APOBEC, or Alu. In some embodiments of the disclosure, the RNA editing entity can have an ADAR protein, an APOBEC protein, or both. When the RNA editing entity is an ADAR protein, the ADAR protein can be selected from the group consisting of: an ADAR1, an ADAR2, and a combination of ADAR1 and ADAR2. Other embodiments can be directed to an RNA editing entity selected from the group consisting of: a human ADAR1, a mouse ADAR1, a human ADAR2, a mouse ADAR2, and any combination thereof.

In some examples, more than one recruiting domain can be included in an engineered guide RNA of the disclosure. In examples where a recruiting domain can be present, the recruiting domain can be utilized to position the RNA editing entity to effectively react with a target RNA after the targeting sequence, for example an antisense sequence, hybridizes to a target RNA. In some cases, a recruiting domain can allow for transient binding of the RNA editing entity to the engineered guide RNA. In some examples, the recruiting domain allows for permanent binding of the RNA editing entity to the engineered guide RNA. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity to GUGGAAUAGUAUAACAAUAUGCUAAAUGUUGUUAUAGUAUCCCAC (SEQ ID NO: 9), a length to: GUGGAAUAGUAUAACAAUAUGCUAAAUGUUGUUAUAGUAUCCCAC (SEQ ID NO: 9), or both. In some cases, a recruiting domain can comprise at least about 80% sequence homology to at least about 10, 15, 20, 25, or 30 nucleotides of SEQ ID NO: 9. In some examples, a recruiting domain can comprise at least about 90%, 95%, 96%, 97%, 98%, or 99% sequence homology to SEQ ID NO: 9, length to SEQ ID NO: 9, or combinations thereof.

Additional RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered RNA of the present disclosure. In some examples, at least about 1, 2, 3, 4, 5, 6, 7, 8, 9, or up to about 10 recruiting domains can be included in an engineered RNA. Recruiting domains can be located at any position of an engineered guide RNA. In some cases, a recruiting domain can be on an N-terminus, middle, or C-terminus of a polynucleotide. A recruiting domain can be upstream or downstream of a targeting sequence. In some cases, a recruiting domain flanks a targeting sequence of a guide. A recruiting sequence can comprise all ribonucleotides or deoxyribonucleotides, although a recruiting domain comprising both ribo- and deoxy-ribonucleotides can in some cases not be excluded.

Engineered Guide RNAs with Latent Structure

In some examples, the engineered guide RNAs disclosed herein can lack a recruiting domain that is formed and present in the absence of hybridization of the engineered guide RNA to the target RNA. In some cases, recruitment of the RNA editing entity can be effectuated by the guide-target RNA scaffold formed by hybridization of the engineered guide RNA and a target RNA. In some examples, the engineered guide RNA, when present in an aqueous solution and not bound to the target RNA molecule, does not comprise structural features that recruit the RNA editing entity (e.g., ADAR, APOBEC, or both). Some embodiments provide for an RNA editing entity comprising an ADAR protein, where the ADAR protein can be selected from the group consisting of an ADAR1 (e.g., human or mouse), an ADAR2 (e.g., human or mouse), and any combination thereof. The engineered guide RNA, upon hybridization to a target RNA, forms with the target RNA, one or more structural features that recruits an RNA editing entity (e.g., ADAR).

In cases where a recruiting sequence can be absent, an engineered guide RNA can still be capable of associating with an RNA editing entity (e.g., ADAR) to facilitate editing of a target RNA, modulate expression of a polypeptide encoded by a target RNA, or combinations thereof. This can be achieved through features constructed in the guide-target RNA scaffold formed upon hybridization of the engineered guide RNA and the target RNA described here. The term “latent structure” refers to a structural feature that substantially forms only upon hybridization of an engineered guide RNA to a target RNA. For example, the sequence of an engineered guide RNA provides one or more structural features, but these structural features substantially form only upon hybridization to the target RNA, and thus the one or more latent structural features manifest as structural features upon hybridization to the target RNA. Upon hybridization of the engineered guide RNA to the target RNA, the structural feature is formed, and the latent structure provided in the engineered guide RNA is, thus, unmasked. A latent guide RNA as described here refers to an engineered guide RNA that comprises a portion of sequence that, upon hybridization to a target RNA, forms at least a portion of a structural feature, other than a single A/C mismatch feature at the target adenosine to be edited. In some embodiments, the targeting sequence structural features can comprise any one of a: mismatch, symmetrical bulge, asymmetrical bulge, symmetrical internal loop, asymmetrical internal loop, hairpins, wobble base pairs, or any combination thereof.

Provided herein are engineered guide RNAs and compositions comprising said engineered guide RNAs. In some examples, the engineered guide RNA can be an engineered polynucleotide. For example, in some embodiments, the present disclosure provides for engineered polynucleotides encoding for engineered RNAs (e.g., engineered guide RNAs, antisense oligonucleotides). In some embodiments, the engineered RNA comprises DNA. In some examples, the engineered RNA comprises modified RNA bases or unmodified RNA bases. In some embodiments, the engineered RNA comprises modified DNA bases or unmodified DNA bases. In some examples, the engineered RNA comprises both DNA and RNA bases.

In some examples, the engineered RNAs (e.g., engineered guide RNAs, antisense oligonucleotides) provided here comprise an engineered RNA that can be configured, upon hybridization to a target RNA or at least a portion of the target RNA, to form, at least in part, a guide-target RNA scaffold. In some embodiments, a guide-target RNA scaffold is formed upon hybridization of an engineered RNA (e.g., engineered guide RNA, antisense oligonucleotide) of the present disclosure to a target RNA. A guide-target RNA scaffold can have structural features formed within a double stranded RNA duplex. For example, the guide-target RNA scaffold can have at least one structural feature, or two or more structural features selected from the group consisting of a mismatch, a bulge (e.g., symmetrical bulge or asymmetrical bulge), an internal loop (e.g., symmetrical internal loop or asymmetrical internal loop), a hairpin (e.g., a recruiting hairpin or a hairpin comprising a non-targeting domain), a wobble base pair, and any combination thereof, where the guide-target RNA scaffold recruits an RNA editing entity and facilitates a chemical modification of a base of a nucleotide in the target RNA by the RNA editing entity.

Described herein can be a structural feature that can be present in a guide-target RNA scaffold of the present disclosure. The guide-target RNA scaffold can be formed upon hybridization of an engineered guide RNA and a target RNA, and the scaffold can have at least one or two or more structural features. Examples of structural features include a mismatch, a bulge (symmetrical bulge or asymmetrical bulge), an internal loop (symmetrical internal loop or asymmetrical internal loop), or a hairpin (a hairpin comprising a non-targeting domain), or wobble base pair. Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. As disclosed herein, a “structured motif” refers to a combination of or comprises two or more features in a guide-target RNA scaffold.

In some examples, a double stranded RNA (dsRNA) substrate (a guide-target RNA scaffold) is formed upon hybridization of an engineered guide RNA of the present disclosure to a target RNA. As disclosed herein, a “mismatch,” as used here, refers to a single nucleotide in an engineered RNA (e.g., engineered guide RNA, ASO) of the disclosure that is unpaired to an opposing single nucleotide in a target RNA within the guide-target RNA scaffold formed upon hybridization of the engineered guide RNA of the present disclosure and the target RNA. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch can be an A/C mismatch, an A/G mismatch, or an A/A mismatch. For example, an A/C mismatch can comprise a C in an engineered RNA (e.g., engineered guide RNA, ASO) of the present disclosure opposite an A in a target RNA. An A/C mismatch can comprise an A in an engineered RNA (e.g., engineered guide RNA, ASO) of the present disclosure opposite a C in a target RNA. For example, a G/G mismatch can comprise a G in an engineered RNA (e.g., engineered guide RNA, ASO) of the present disclosure opposite a G in a target RNA. In some embodiments, a mismatch positioned 5′ of the edit site can facilitate base-flipping of the A of the target RNA (or target A) to be edited. A mismatch can also assist in conferring sequence specificity. Thus, a mismatch can be a structural feature formed from a latent structure provided by an engineered latent guide RNA. In some embodiments, a mismatch comprises an A/C mismatch, wherein the A can be in the target RNA and the C can be in the targeting sequence of the engineered RNA (e.g., engineered guide RNA, antisense oligonucleotide). In another embodiment, the A in the A/C mismatch can be the base of the nucleotide in the target RNA edited by an RNA editing entity.

A double stranded RNA (dsRNA) substrate (a guide-target RNA scaffold) is formed upon hybridization of an engineered guide RNA of the present disclosure to a target RNA. In some aspects, a “bulge” refers to the structure substantially formed only upon formation of the guide-target RNA scaffold, where contiguous nucleotides in either the engineered guide RNA of the disclosure or the target RNA are not complementary to their positional counterparts on the opposite strand. A bulge can change the secondary or tertiary structure of the guide-target RNA scaffold. A bulge can have from 0 to 4 contiguous nucleotides on the engineered guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the engineered guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the engineered guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some examples, the presence of a bulge in a guide-target RNA scaffold can recruit or help recruit additional amounts of ADAR proteins (e.g., mouse or human ADAR1, mouse or human ADAR2, or any combination thereof). Bulges in guide-target RNA scaffolds disclosed here can recruit other proteins, such as other RNA editing entities (e.g., an Apolipoprotein B mRNA Editing Catalytic Polypeptide-like (APOBEC), or both an ADAR and an APOBEC). In some embodiments, a bulge positioned 5′ of the edit site can facilitate base-flipping of the target “A” of the target RNA to be edited. A bulge can also assist in conferring sequence specificity for the A of the target RNA to be edited, relative to other A(s) present in the target RNA. For example, a bulge can assist in directing ADAR editing by constraining it in an orientation that yields selective editing of the target A of the target RNA.

In some aspects, a guide-target RNA scaffold is formed upon hybridization of an engineered guide RNA of the present disclosure to a target RNA. A bulge of the disclosure can be a symmetrical bulge or an asymmetrical bulge. A “symmetrical bulge” is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold and 2 nucleotides on the target RNA side of the guide-target RNA scaffold. A symmetrical bulge of the present disclosure can be formed by 3 nucleotides on the engineered guide RNA side of the guide-target RNA scaffold and 3 nucleotides on the target RNA side of the guide-target RNA scaffold. A symmetrical bulge of the present disclosure can be formed by 4 nucleotides on the engineered guide RNA side of the guide-target RNA scaffold and 4 nucleotides on the target RNA side of the guide-target RNA scaffold. Thus, a symmetrical bulge can be a structural feature formed from latent structure provided by an engineered latent guide RNA.

In some examples, a double stranded RNA (dsRNA) substrate (a guide-target RNA scaffold) is formed upon hybridization of an engineered guide RNA of the present disclosure to a target RNA. A bulge can be a symmetrical or an asymmetrical bulge. An “asymmetrical bulge” is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. In some embodiments, an asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some examples, a double stranded RNA (dsRNA) substrate (a guide-target RNA scaffold) is formed upon hybridization of an engineered guide RNA of the present disclosure to a target RNA. As disclosed here, an “internal loop” refers to the structure substantially formed upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the engineered guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can assist with base flipping of the target A in the target RNA to be edited.

In some aspects, a guide-target RNA scaffold can be formed upon hybridization of an engineered RNA (e.g., engineered guide RNA, antisense oligonucleotides) of the present disclosure to a target RNA. One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1,000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1,000 nucleotides. Thus, an internal loop can be a structural feature formed from latent structure provided by an engineered latent guide RNA.

In some embodiments, a double stranded RNA (dsRNA) substrate (a guide-target RNA scaffold) is formed upon hybridization of an engineered guide RNA of the present disclosure to a target RNA. An internal loop can be a symmetrical internal loop or an asymmetrical internal loop. A “symmetrical internal loop” can be formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. In some examples described here, a symmetrical internal loop of the present disclosure can be formed from 5 to 150 nucleotides on the engineered guide RNA side of the guide-target RNA scaffold and from 5 to 150 nucleotides on the target RNA side of the guide-target RNA scaffold, where the number of nucleotides is the same on the engineered guide RNA side of the guide-target RNA scaffold and on the target RNA side of the guide-target RNA scaffold. A symmetrical internal loop of the present disclosure can be formed from 5 to 1000 nucleotides on the engineered guide RNA side of the dsRNA target and from 5 to 1000 nucleotides on the target RNA side of the guide-target RNA scaffold, wherein the number of nucleotides is the same on the engineered guide RNA side of the guide-target RNA scaffold and on the target RNA side of the guide-target RNA scaffold. A symmetrical internal loop of the present disclosure can be formed by 5 nucleotides on the engineered guide RNA side of the guide-target RNA scaffold and 5 nucleotides on the target RNA side of the guide-target RNA scaffold. A symmetrical internal loop of the present disclosure can be formed by 6 nucleotides on the engineered guide RNA side of the guide-target RNA scaffold and 6 nucleotides on the target RNA side of the guide-target RNA scaffold. A symmetrical internal loop of the present disclosure can be formed by 7 nucleotides on the engineered guide RNA side of the guide-target RNA scaffold and 7 nucleotides on the target RNA side of the guide-target RNA scaffold. A symmetrical internal loop of the present disclosure can be formed by 8 nucleotides on the engineered guide RNA side of the guide-target RNA scaffold and 8 nucleotides on the target RNA side of the guide-target RNA scaffold. A symmetrical internal loop of the present disclosure can be formed by 9 nucleotides on the engineered guide RNA side of the guide-target RNA scaffold and 9 nucleotides on the target RNA side of the guide-target RNA scaffold. A symmetrical internal loop of the present disclosure can be formed by 10 nucleotides on the engineered guide RNA side of the guide-target RNA scaffold and 10 nucleotides on the target RNA side of the guide-target RNA scaffold. A symmetrical internal loop of the present disclosure can be formed by 15 nucleotides on the engineered guide RNA side of the guide-target RNA scaffold and 15 nucleotides on the target RNA side of the guide-target RNA scaffold. A symmetrical internal loop of the present disclosure can be formed by 20 nucleotides on the engineered guide RNA side of the guide-target RNA scaffold and 20 nucleotides on the target RNA side of the guide-target RNA scaffold. A symmetrical internal loop of the present disclosure can be formed by 30 nucleotides on the engineered guide RNA side of the guide-target RNA scaffold and 30 nucleotides on the target RNA side of the guide-target RNA scaffold. A symmetrical internal loop of the present disclosure can be formed by 40 nucleotides on the engineered guide RNA side of the guide-target RNA scaffold and 40 nucleotides on the target RNA side of the guide-target RNA scaffold. A symmetrical internal loop of the present disclosure can be formed by 50 nucleotides on the engineered guide RNA side of the guide-target RNA scaffold and 50 nucleotides on the target RNA side of the guide-target RNA scaffold. A symmetrical internal loop of the present disclosure can be formed by 60 nucleotides on the engineered guide RNA side of the guide-target RNA scaffold and 60 nucleotides on the target RNA side of the guide-target RNA scaffold. A symmetrical internal loop of the present disclosure can be formed by 70 nucleotides on the engineered guide RNA side of the guide-target RNA scaffold and 70 nucleotides on the target RNA side of the guide-target RNA scaffold. A symmetrical internal loop of the present disclosure can be formed by 80 nucleotides on the engineered guide RNA side of the guide-target RNA scaffold and 80 nucleotides on the target RNA side of the guide-target RNA scaffold. A symmetrical internal loop of the present disclosure can be formed by 90 nucleotides on the engineered guide RNA side of the guide-target RNA scaffold and 90 nucleotides on the target RNA side of the guide-target RNA scaffold. A symmetrical internal loop of the present disclosure can be formed by 100 nucleotides on the engineered guide RNA side of the guide-target RNA scaffold and 100 nucleotides on the target RNA side of the guide-target RNA scaffold. A symmetrical internal loop of the present disclosure can be formed by 110 nucleotides on the engineered guide RNA side of the guide-target RNA scaffold and 110 nucleotides on the target RNA side of the guide-target RNA scaffold. A symmetrical internal loop of the present disclosure can be formed by 120 nucleotides on the engineered guide RNA side of the guide-target RNA scaffold and 120 nucleotides on the target RNA side of the guide-target RNA scaffold. A symmetrical internal loop of the present disclosure can be formed by 130 nucleotides on the engineered guide RNA side of the guide-target RNA scaffold and 130 nucleotides on the target RNA side of the guide-target RNA scaffold. A symmetrical internal loop of the present disclosure can be formed by 140 nucleotides on the engineered guide RNA side of the guide-target RNA scaffold and 140 nucleotides on the target RNA side of the guide-target RNA scaffold. A symmetrical internal loop of the present disclosure can be formed by 150 nucleotides on the engineered guide RNA side of the guide-target RNA scaffold and 150 nucleotides on the target RNA side of the guide-target RNA scaffold. A symmetrical internal loop of the present disclosure can be formed by 200 nucleotides on the engineered guide RNA side of the guide-target RNA scaffold and 200 nucleotides on the target RNA side of the guide-target RNA scaffold. A symmetrical internal loop of the present disclosure can be formed by 250 nucleotides on the engineered guide RNA side of the guide-target RNA scaffold and 250 nucleotides on the target RNA side of the guide-target RNA scaffold. A symmetrical internal loop of the present disclosure can be formed by 300 nucleotides on the engineered guide RNA side of the guide-target RNA scaffold and 300 nucleotides on the target RNA side of the guide-target RNA scaffold. A symmetrical internal loop of the present disclosure can be formed by 350 nucleotides on the engineered guide RNA side of the guide-target RNA scaffold and 350 nucleotides on the target RNA side of the guide-target RNA scaffold. A symmetrical internal loop of the present disclosure can be formed by 400 nucleotides on the engineered guide RNA side of the guide-target RNA scaffold and 400 nucleotides on the target RNA side of the guide-target RNA scaffold. A symmetrical internal loop of the present disclosure can be formed by 450 nucleotides on the engineered guide RNA side of the guide-target RNA scaffold and 450 nucleotides on the target RNA side of the guide-target RNA scaffold. A symmetrical internal loop of the present disclosure can be formed by 500 nucleotides on the engineered guide RNA side of the guide-target RNA scaffold and 500 nucleotides on the target RNA side of the guide-target RNA scaffold. A symmetrical internal loop of the present disclosure can be formed by 600 nucleotides on the engineered guide RNA side of the guide-target RNA scaffold and 600 nucleotides on the target RNA side of the guide-target RNA scaffold. A symmetrical internal loop of the present disclosure can be formed by 700 nucleotides on the engineered guide RNA side of the guide-target RNA scaffold and 700 nucleotides on the target RNA side of the guide-target RNA scaffold. A symmetrical internal loop of the present disclosure can be formed by 800 nucleotides on the engineered guide RNA side of the guide-target RNA scaffold and 800 nucleotides on the target RNA side of the guide-target RNA scaffold. A symmetrical internal loop of the present disclosure can be formed by 900 nucleotides on the engineered guide RNA side of the guide-target RNA scaffold and 900 nucleotides on the target RNA side of the guide-target RNA scaffold. A symmetrical internal loop of the present disclosure can be formed by 1,000 nucleotides on the engineered guide RNA side of the guide-target RNA scaffold and 1,000 nucleotides on the target RNA side of the guide-target RNA scaffold. Thus, a symmetrical internal loop can be a structural feature formed from latent structure provided by an engineered latent guide RNA.

Some examples of the disclosure provide a guide-target RNA scaffold that can be formed upon hybridization of an engineered guide RNA of the present disclosure to a target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold. In some aspects, an asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, where the number of nucleotides is different on the engineered guide RNA side of the guide-target RNA scaffold than the number of nucleotides on the target RNA side of the guide-target RNA scaffold. An asymmetrical internal loop of the present disclosure can be formed from 5 to 1,000 nucleotides on the engineered guide RNA side of the guide-target RNA scaffold and from 5 to 1,000 nucleotides on the target RNA side of the guide-target RNA scaffold, where the number of nucleotides is different on the engineered guide RNA side of the guide-target RNA scaffold than the number of nucleotides on the target RNA side of the guide-target RNA scaffold. In some examples, an asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

Some embodiments provide a guide-target RNA scaffold formed upon hybridization of an engineered guide RNA of the present disclosure and a target RNA, where a structural feature can be present in the guide-target RNA scaffold of the present disclosure, and the structural feature can be a hairpin. In some cases, an engineered guide RNA of the disclosure can lack a hairpin domain. In other cases, an engineered guide RNA described here can contain a hairpin domain or more than one hairpin domain. A “hairpin” as described here includes an RNA duplex, where a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base-paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed here. The engineered guide RNAs disclosed here can have from 1 to 10 hairpins. In some embodiments, the engineered guide RNAs disclosed here can have 1 hairpin. In some embodiments, the engineered guide RNAs disclosed here can have 2 hairpins. As disclosed here, a hairpin can refer to a recruitment hairpin or a hairpin or a non-recruitment hairpin. A hairpin can be located anywhere within the engineered guide RNAs of the present disclosure. In some embodiments, one or more hairpins is proximal to or present at the 3′ end of an engineered RNA of the present disclosure, proximal to or at the 5′ end of an engineered guide RNAs of the present disclosure, or any combination thereof.

In some examples, a “recruitment hairpin,” as disclosed here, can recruit at least in part an RNA editing entity, such as ADAR. In some embodiments, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined here, can include a naturally occurring ADAR substrate or truncations thereof. Thus, a recruitment hairpin such as GluR2 is a pre-formed structural feature that may be present in constructs comprising an engineered guide RNA, not a structural feature formed by latent structure provided in an engineered latent guide RNA

In some aspects, a “non-recruitment hairpin,” as disclosed here, does not have a primary function of recruiting an RNA editing entity. A non-recruitment hairpin, in some instances, does not recruit an RNA editing entity. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that may be present in constructs comprising engineered guide RNA constructs, not a structural feature formed by latent structure provided in an engineered latent guide RNA.

A hairpin of the present disclosure can be of any length. In some aspects, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

In other aspects, a structural feature described here comprises a wobble base. A “wobble base pair” refers to two bases that weakly base pair. For example, a wobble base pair of the present disclosure can refer to a G paired with a U. Thus, a wobble base pair can be a structural feature formed from latent structure provided by an engineered latent guide RNA.

### RNA Editing

RNA editing can refer to a process by which RNA can be enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from cytidine to give rise to uridine, or C-to-U editing or from adenosine to inosine, or A-to-I editing). RNA editing can be used to introduce mutations, correct missense mutations, or edit coding or non-coding regions of RNA to inhibit RNA translation and effect protein knockdown. In some instances, inhibiting, covering, masking, or blocking a target sequence in a target RNA using an engineered RNA of the present disclosure can be used to knock down expression of protein translated from the target RNA.

The engineered RNAs (e.g., engineered guide RNA or ASO) of the present disclosure comprising a targeting sequence can be linked to a heterologous engineered RNA element (e.g., an engineered SmOPT variant, an engineered U7 hairpin variant, or both such as an element having a polynucleotide sequence of any one of SEQ ID NO: 49, SEQ ID NO: 50, SEQ ID NO: 51, SEQ ID NO: 60, SEQ ID NO: 61, or SEQ ID NO: 62). The engineered guide RNAs described here are longer in length than the antisense oligonucleotides. Accordingly, the engineered guide RNAs are capable of much higher degrees of structure as compared to shorter chemically modified guides or antisense oligonucleotides that cannot generate the same structures as those by the engineered guide RNAs. In some embodiments, the engineered RNAs (e.g., engineered guide RNA or ASO) of the disclosure comprising a targeting sequence substantially complementary to a target RNA can be operably linked to an engineered SmOPT variant sequence described here. In other examples, the engineered RNAs (e.g., engineered guide RNA or ASO) of the disclosure comprising a targeting sequence substantially complementary to a target RNA can be operably linked to an engineered U7 hairpin variant sequence described here. Yet in further embodiments of the disclosure, the described engineered RNAs (e.g., guide RNA or ASO) comprising a targeting sequence substantially complementary to a target RNA can be operably linked to an engineered SmOPT variant sequence and an engineered U7 hairpin variant sequence described here.

In some instances, RNA editing entities, such as but not limited to ADARs, can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of human ADAR genes (e.g., ADARs 1-3). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. The engineered guide RNAs of the present disclosure facilitate RNA editing by endogenous ADAR enzymes. In some embodiments, exogenous ADAR can be delivered alongside the engineered guide RNAs disclosed herein.

As provided herein, an engineered guide RNA can comprise a targeting sequence with target complementarity to a target RNA of interest. Hybridization of the target RNA to the targeting sequence of an engineered guide RNA can provide a structural feature that is a substrate for ADAR-editing. Accordingly, engineered guide RNA as provided herein can site-specifically bind targets and facilitate targeted editing of target RNA. Further, an engineered guide RNA as described herein can comprise two additional sequences that, when present, increase an amount or efficiency of editing. First, an engineered guide RNA can comprise a Sm or Sm-like binding domain sequence. Second, an engineered guide RNA can comprise a hairpin from an snRNA. Without wishing to be bound by theory, the presence of one of both of these features can improve localization of the engineered guide RNA to the target RNA. Further, an engineered guide RNA of the present disclosure can include engineered Sm or Sm-like binding domains in which at least one nucleotide is substituted relative to a naturally-occurring Sm or Sm-like binding domain. Further, an engineered RNA of the present disclosure can include snRNA hairpins in which at least one nucleotide is substituted relative to a naturally-occurring snRNA hairpin. For example, engineered guide RNAs of the disclosure can comprise a targeting sequence with sufficient complementarity to a target RNA allowing for hybridization of the engineered guide RNA and the target RNA, where the engineered guide RNA is operably linked to an RNA element, such as, an engineered SmOPT variant sequence comprising an altered or variant of an Sm binding domain sequence of SEQ ID NO: 1 (AAUUUGUSKAG) or an SmOPT sequence of SEQ ID NO: 2 (AAUUUUUGGAG); an engineered U7 hairpin variant sequence comprising an altered or variant of a U7 hairpin sequence of SEQ ID NO: 3 (mouse: CAGGUUUUCUGACUUCGGUCGGAAAACCCCU) or SEQ ID NO: 4 (human: UAGGCUUUCUGGCUUUUUACCGGAAAGCCCCU) as described here, or combinations thereof.

The described engineered guide RNAs of the disclosure can further be operably linked to an RNA polymerase II-type promoter in some embodiments. Non-limiting examples of useful RNA polymerase II-type promoters of the disclosure include: a U1 promoter (e.g., SEQ ID NO: 10; human); a U7 promoter (e.g., SEQ ID NO: 11—mouse; SEQ ID NO: 12—human), and any combination thereof. In some embodiments, the described engineered guide RNA can be operably linked to a U7 promoter. Some examples can provide for the described engineered guide RNA comprising: a targeting sequence of (a); an engineered SmOPT variant sequence of (b), where the engineered SmOPT variant sequence is an engineered Sm or Sm-like protein binding domain, where the engineered Sm or Sm-like protein binding domain variant sequence can be an engineered SmOPT variant sequence; an engineered U7 hairpin variant sequence of (c), where the engineered U7 hairpin variant sequence is an engineered mouse U7 snRNA hairpin variant sequence or an engineered human U7 snRNA hairpin variant sequence, or both the engineered SmOPT variant sequence of (b) and the engineered U7 hairpin variant sequence of (c).

In some embodiments, the described engineered guide RNA further comprises a terminator. An exemplary terminator includes, but is not limited to, a murine U7 terminator (SEQ ID NO: 13), a human U7 terminator (SEQ ID NO: 14), or a U7 box terminator, where the terminator can be operably linked to the engineered guide RNA. In some embodiments, a terminator comprises a 3′ box. In some embodiments, a terminator is a 3′ box. A 3′ box can be, but is not limited to, a mouse U7 3′ box (mU7 3′ box; SEQ ID NO: 15) or a human U7 3′ box (hU7 3′ box; SEQ ID NO: 16). In some embodiments, the disclosed engineered RNA further comprises a terminator that is 3′ of the at least one mismatch formed upon hybridization of the targeting sequence and the target RNA. In some embodiments, the terminator is 3′ to the hairpin as disclosed herein. In some embodiments, the terminator comprises a 3′ box, one or more nucleotides positioned between the 3′ box and the hairpin, and one or more nucleotides that are 3′ to the 3′ box. In some embodiments, the terminator comprises a 3′ box and one or more nucleotides positioned between the 3′ box and the hairpin.

In some embodiments, the terminator is a truncated terminator. A truncated terminator can be a terminator having at least one nucleotide less than the reference terminator. A reference terminator for a truncated terminator is, for example, a murine U7 terminator (SEQ ID NO: 13), a human U7 terminator (SEQ ID NO: 14), or a U7 box terminator. In some embodiments, a truncation of the truncated terminator is from 1 to 150 nucleotides less than the reference terminator. In some embodiments, the truncation is 50 nucleotides less than the reference terminator. In some embodiments, the truncation is 79 nucleotides less than the reference terminator. In some embodiments, the truncation is 92 nucleotides less than the reference terminator. In some embodiments, the truncation is of one or more nucleotides positioned between a hairpin and the 3′ box as compared to the reference terminator. In some embodiments, the truncation is of one or more nucleotides positioned 3′ of the 3′ box as compared to the reference terminator. In some embodiments, the truncation is of one or more nucleotides positioned between a hairpin and the 3′ box as compared to the reference terminator and one or more nucleotides positioned 3′ of the 3′ box as compared to the reference terminator. For example, a truncated terminator has a deletion of 50 nucleotides that are 3′ to the 3′ box as compared to the reference terminator (e.g., SEQ ID NO: 14). As another example, a truncated terminator has a deletion of 50 nucleotides that are 3′ to the 3′ box as compared to the reference terminator (e.g., SEQ ID NO: 14) and a deletion of 28 nucleotides that are positioned between the hairpin and the 3′ box as compared to the reference terminator (e.g., SEQ ID NO: 14). In some embodiments, the hairpin is truncated compared to reference hairpin, such as a sequence of a hairpin as disclosed herein. In some embodiments, the truncated hairpin has a deletion of from 1 to 15 nucleotides as compared to a reference hairpin. In some embodiments, the truncated hairpin has a deletion of 7 nucleotides as compared to a reference hairpin. Various elements of the engineered guide RNAs are illustrated in TABLE 2.

In some embodiments, the engineered guide RNA of the disclosure targets (a) a RAB7A 3′UTR that can be expressed using a U7 or U1 promoter, such as but not limited to, a mouse U7 (mU7) promoter, a human U7 (hU7) promoter, or human U1 (hU1) promoter), with (b) an engineered SmOPT variant sequence and (c) an engineered U7 hairpin variant sequence, and operably linked to a mouse U7 (mU7) terminator sequence or human U7 (hU7) terminator sequence. In some embodiments, the mouse U7 or human U7 terminator is a truncated terminator.

Other aspects described here provide for an engineered guide RNA of the disclosure, which targets (a) a RAB7A exon 1 that can be expressed using a mU7 promoter or a RAB7A exon 3 human U7 promoter, with (b) an engineered SmOPT variant sequence, (c) an engineered U7 hairpin variant sequence, or both the engineered SmOPT variant sequence of (b) and the engineered U7 hairpin variant sequence of (c), and operably linked to a mouse U7 or human U7 terminator sequence. In some embodiments, the mouse U7 or human U7 terminator is a truncated terminator.

Other examples can provide for an engineered RNA of the disclosure, where the engineered RNA is an engineered guide RNA, which targets (a) an LRRK2 gene that can be expressed using any promoter (e.g., U1, U6, or U7), with (b) an engineered SmOPT variant sequence, (c) an engineered U7 hairpin variant sequence, or both the engineered SmOPT variant sequence of (b) and the engineered U7 hairpin variant sequence of (c), and operably linked to a mouse U7 terminator sequence or a human U7 terminator sequence. In some embodiments, the mouse U7 terminator sequence or human U7 terminator sequence is a truncated terminator.

In some embodiments, the engineered RNA comprises an engineered guide RNA, which targets (a) an ABCA4 gene that can be expressed using any promoter, such as for example, a U6 promoter or a U1 promoter, with (b) a sequence or an engineered SmOPT variant sequence, (c) a hairpin or an engineered U7 hairpin variant sequence, or both the engineered SmOPT variant sequence of (b) and the engineered U7 hairpin variant sequence of (c), where at least one of (b) and (c) is engineered, and operably linked to a terminator sequence (e.g., mouse U7 or human U7 terminator sequence). In some embodiments, the mouse U7 or human U7 terminator is a truncated terminator.

In some examples, an engineered RNA of the disclosure comprises an engineered guide RNA, which targets (a) a RAB7A 3′UTR that can be expressed using, for example, a human U1 promoter with a 5′ double hnRNP A1 binding domain, with (b) an engineered SmOPT variant sequence, (c) an engineered U7 hairpin variant sequence, or both the engineered SmOPT variant sequence of (b) and the engineered U7 hairpin variant sequence of (c), and operably linked to a terminator sequence, for example, a mouse U7 or human U7 terminator sequence. In some embodiments, the mouse U7 or human U7 terminator is a truncated terminator.

Some embodiments provide for in vitro assays where an editing efficiency can be determined. In some aspects, the in vitro assay for determining RNA levels or an amount of editing of a base of a nucleotide of the target RNA by an RNA editing entity with the engineered guide RNAs described here includes RNA sequencing. For example, an engineered guide RNA comprising an engineered SmOPT variant sequence, an engineered U7 hairpin variant sequence, or both can facilitate an increase in an amount of editing of a base of a nucleotide of a target RNA by an RNA editing entity, relative to an otherwise comparable RNA lacking the engineered SmOPT variant sequence, the engineered U7 hairpin variant sequence, or both the engineered SmOPT variant sequence and the engineered U7 hairpin variant sequence, as determined by RNA sequencing. Some examples provide for in vitro assays where an editing efficiency can be determined by (i) transfecting a target RNA into a primary cell line, (ii) transfecting an engineered polynucleotide and an otherwise comparable polynucleotide into a primary cell line, and (iii) sequencing the target RNA. In some embodiments, an editing efficiency can be determined by (i) transfecting a target RNA into a primary cell line, (ii) transfecting an engineered polynucleotide and an otherwise comparable polynucleotide into a primary cell line, and (iii) mass spectroscopy of the target RNA. In some embodiments, an edit of a base of a nucleotide of a target RNA by an RNA editing entity can be determined in an in vitro assay comprising: (i) directly or indirectly introducing (e.g., transfecting) the target RNA into a primary cell line, (ii) directly or indirectly introducing (e.g., transfecting) the engineered polynucleotide into a primary cell line, and (iii) sequencing the target RNA. In some cases, transfecting the target RNA into a primary cell line can comprise transfecting a plasmid encoding for the target RNA into a primary cell line. In some instances, transfecting an engineered polynucleotide into a primary cell line can comprise transfecting a precursor engineered polynucleotide, or a polynucleotide (e.g., plasmid) that encodes for a precursor engineered polynucleotide into a primary cell line. In some cases, sequencing can comprise Sanger sequencing of a target RNA after the target RNA has been converted to cDNA by reverse transcriptase.

In some embodiments, the engineered guide RNAs of the present disclosure comprising a targeting sequence sufficiently complementary to a target RNA of interest, and an engineered SmOPT variant sequence, an engineered U7 hairpin variant sequence, or both the engineered SmOPT variant sequence and the engineered U7 hairpin variant sequence, facilitated ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

TABLE 2
Components of the Engineered RNAs of the Description

| | | Sequence (where T is present, |
|---|---|---|
| SEQ ID | | sequences are represented as |
| NO | Name | DNA sequences; K = G or U; S = G or C) |
| SEQ ID | Sm binding domain | AAUUUGUSKAG |
| NO: 1 | sequence | |
| SEQ ID | SmOPT sequence | AAUUUUUGGAG |
| NO: 2 | | |
| SEQ ID | Mouse U7 Hairpin | CAGGUUUUCUGACUUCGGUCGGAAAACCCCU |
| NO: 3 | Sequence | |
| SEQ ID | Human U7 Hairpin | UAGGCUUUCUGGCUUUUUACCGGAAAGCCCCU |
| NO: 4 | Sequence | |
| | Consensus sequence | N₁N₂N₃NAN₅N₆N₇N₈N₉N₁₀N₁₁N₁N₂N₃NAN₅N₆N₇N₈N₉N₁₀N₁₁ |
| | Consensus hairpin | N₁₂N₃N₁₄N₁₅N₁₆N₁₇N₁₈N₁₉N₂₀N₂₁N₂₂N₂₃N₂₄N₂₅N₂₆N₂₇N₂₈ |
| | Consensus Sm | N₂₉N₃₀N₃₁N₃₂ |
| | sequence | |
| SEQ ID | Consensus hairpin | N₁AGGN₂UUUCUGN₃CUUN₄N₅N₆N₇CN₈GN₉AAAN₁₀CCCN₁₁ |
| NO: 8 | sequence | N₁₂ |
| SEQ ID | Human U1 promoter | TAAGGACCAGCTTCTTTGGGAGAGAACAGACGCAGGGG |
| NO: 10 | | CGGGAGGGAAAAAGGGAGAGGCAGACGTCACTTCCTCT |
| | | TGGCGACTCTGGCAGCAGATTGGTCGGTTGAGTGGCAGA |
| | | AAGGCAGACGGGGACTGGGCAAGGCACTGTCGGTGACA |
| | | TCACGGACAGGGCGACTTCTATGTAGATGAGGCAGCGC |
| | | AGAGGCTGCTGCTTCGCCACTTGCTGCTTCGCCACGAAG |
| | | GGAGTTCCCGTGCCCTGGGAGCGGGTTCAGGACCGCTGA |
| | | TCGGAAGTGAGAATCCCAGCTGTGTGTCAGGGCTGGAA |
| | | AGGGCTCGGGAGTGCGCGGGGCAAGTGACCGTGTGTGT |
| | | AAAGAGTGAGGCGTATGAGGCTGTGTCGGGGCAGAGCC |
| | | CGAAGATCTC |
| SEQ ID | Mouse U7 promoter | TTAACAACATAGGAGCTGTGATTGGCTGTTTTCAGCCAA |
| NO: 11 | | TCAGCACTGACTCATTTGCATAGCCTTTACAAGCGGTCA |
| | | CAAACTCAAGAAACGAGCGGTTTTAATAGTCTTTTAGAA |
| | | TATTGTTTATCGAACCGAATAAGGAACTGTGCTTTGTGA |
| | | TTCACATATCAGTGGAGGGGTGTGGAAATGGCACCTTGA |
| | | TCTCACCCTCATCGAAAGTGGAGTTGATGTCCTTCCCTG |
| | | GCTCGCTACAGACGCACTTCCGC |
| SEQ ID | Human U7 promoter | TTAACAACAACGAAGGGGCTGTGACTGGCTGCTTTCTCA |
| NO: 12 | | ACCAATCAGCACCGAACTCATTTGCATGGGCTGAGAACA |
| | | AATGTTCGCGAACTCTAGAAATGAATGACTTAAGTAAGT |
| | | TCCTTAGAATATTATTTTTCCTACTGAAAGTTACCACATG |
| | | CGTCGTTGTTTATACAGTAATAGGAACAAGAAAAAAGTC |
| | | ACCTAAGCTCACCCTCATCAATTGTGGAGTTCCTTTATAT |
| | | CCCATCTTCTCTCCAAACACATACGCA |
| SEQ ID | Mouse U7 terminator | CCCAATTTCACTGGTCTACAATGAAAGCAAAACAGTTCT |
| NO: 13 | | CTTCCCCGCTCCCCGGTGTGTGAGAGGGGCTTTGATCCTT |
| | | CTCTGGTTTCCTAGGAAACGCGTATGTG |
| SEQ ID | Human U7 | CTTATGATGTTTGTTGCCAATGATAGATTGTTTTCACTGT |
| NO: 14 | terminator | GCAAAAATTATGGGTAGTTTTGGTGGTCTTGATGCAGTT |
| | | GTAAGCTTGGGGTATG |
| SEQ ID | Mouse U7 3′box | GTCTACAATGAAAGC |
| NO: 15 | | |
| SEQ ID | Human U7 3′box | TGTTTGTTGCCAATGATAGAT |
| NO: 16 | | |
| SEQ ID | SNCA 3′UTR | AACATCGTAGATTGAAGCCACAAAATCCACAGCACACA |
| NO: 17 | 100@50 | AAGACCCTGCCACCATGTATTCACTTCAGTGAAAGGGAA |
| | | GCACCGAAATGCTGAGTGGGGGC |
| SEQ ID | RAB7A 3′UTR | TATGATAGGGACTTAGGGTGTGATAAAAGGCGTACATA |
| NO: 18 | hnRNPA1 100@50 | ATTCTTGTGTCTACTGTACAGAATACTGCCGCCAGCTGG |
| | | ATTTCCCAATTCTGAGTAACACTCTGCAATCCAAACAGG |
| | | GTTC |
| SEQ ID | GAPDH 115@80 4 | TGGCAGTGATGGCATGGACTGTGGTCATGAGTCCTTCCA |
| NO: 19 | loops (−5, +30) | CGATACGTTCGTTGTCATGGATGACCTTGGCCAGGGGTG |
| | | CCAAGCACAACGTGGTGCAGGAGGCATTGCTGATGAT |
| SEQ ID | U1Sm RNA | AAUUUGUGGAG |
| NO: 20 | sequence | |
| SEQ ID | U7Sm RNA | AAUUUGUCUAG |
| NO: 21 | sequence | |
| SEQ ID | SmOPT DNA | AATTTTTGGAG |
| NO: 22 | sequence | |
| SEQ ID | U1Sm DNA | AATTTGTGGAG |
| NO: 23 | Sequence | |
| SEQ ID | U7Sm DNA | AATTTGTCTAG |
| NO: 24 | sequence | |
| SEQ ID | Partial mU7 hairpin | CAGGTTTTCTGAC |
| NO: 25 | DNA sequence | |
| SEQ ID | Partial mU7 hairpin | CAGGUUUUCUGAC |
| NO: 26 | RNA sequence | |
| SEQ ID | Partial hU7 hairpin | TAGGCTTTCTGG |
| NO: 27 | DNA sequence | |
| SEQ ID | Partial hU7 hairpin | UAGGCUUUCUGG |
| NO: 28 | RNA sequence | |
| SEQ ID | SmOPT-OT with | TAATTTTTGGAGCAGGTTTTCTGACTTCGGTCGGAA |
| NO: 29 | mU7 hairpin | AACCCCT |
| SEQ ID | SmOPT-11A with | AATTTTTGGAACAGGTTTTCTGACTTCGGTCGGAAA |
| NO: 30 | mU7 hairpin | ACCCCT |
| SEQ ID | SmOPT-11C with | AATTTTTGGACCAGGTTTTCTGACTTCGGTCGGAAA |
| NO: 31 | mU7 hairpin | ACCCCT |
| SEQ ID | SmOPT-11T with | AATTTTTGGATCAGGTTTTCTGACTTCGGTCGGAAA |
| NO: 32 | mU7 hairpin | ACCCCT |
| SEQ ID | SmOPT with mU7- | AATTTTTGGAGCTGGTTTTCTGACTTCGGTCGGAAA |
| NO: 33 | 2T hairpin | ACCCCT |
| SEQ ID | SmOPT with mU7- | AATTTTTGGAGCAGGGTTTTCTGACTTCGGTCGGAA |
| NO: 34 | 3GG hairpin | AACCCCCT |
| SEQ ID | SmOPT with mU7- | AATTTTTGGAGCAGGATTTCTGACTTCGGTCGGAAA |
| NO: 35 | 5A hairpin | TCCCCT |
| SEQ ID | SmOPT with mU7- | AATTTTTGGAGCAGGTCTTCTGACTTCGGTCGGAAG |
| NO: 36 | 6C hairpin | ACCCCT |
| SEQ ID | SmOPT with mU7- | AATTTTTGGAGCAGGTTTTCAGACTTCGGTCTGAAA |
| NO: 37 | 10A hairpin | ACCCCT |
| SEQ ID | SmOPT with mU7- | AATTTTTGGAGCAGGTTTTCTGCCTTCGGGCGGAAA |
| NO: 38 | 12C hairpin | ACCCCT |
| SEQ ID | SmOPT-OU with | UAAUUUUUGGAGCAGGUUUUCUGACUUCGGUCGG |
| NO: 39 | mU7 hairpin RNA | AAAACC CCU |
| | sequence | |
| SEQ ID | SmOPT-11A with | AAUUUUUGGAACAGGUUUUCUGACUUCGGUCGGA |
| NO: 40 | mU7 hairpin RNA | AAACCCCU |
| | sequence | |
| SEQ ID | SmOPT-11C with | AAUUUUUGGACCAGGUUUUCUGACUUCGGUCGGA |
| NO: 41 | mU7 hairpin RNA | AAACCCCU |
| | sequence | |
| SEQ ID | SmOPT-11U with | AAUUUUUGGAUCAGGUUUUCUGACUUCGGUCGGA |
| NO: 42 | mU7 hairpin RNA | AAACCCCU |
| | sequence | |
| SEQ ID | SmOPT with mU7- | AAUUUUUGGAGCUGGUUUUCUGACUUCGGUCGGA |
| NO: 43 | 2U hairpin RNA | AAACCCCU |
| | sequence | |
| SEQ ID | SmOPT with mU7- | AAUUUUUGGAGCAGGGUUUUCUGACUUCGGUCGG |
| NO: 44 | 3GG hairpin RNA | AAAACCCCCU |
| | sequence | |
| SEQ ID | SmOPT with mU7- | AAUUUUUGGAGCAGGAUUUCUGACUUCGGUCGGA |
| NO: 45 | 5A hairpin RNA | AAUCCCCU |
| | sequence | |
| SEQ ID | SmOPT with mU7- | AAUUUUUGGAGCAGGUCUUCUGACUUCGGUCGGA |
| NO: 46 | 6C hairpin RNA | AGACCCCU |
| | sequence | |
| SEQ ID | SmOPT with mU7- | AAUUUUUGGAGCAGGUUUUCAGACUUCGGUCUGA |
| NO: 47 | 10A hairpin RNA | AAACCCCU |
| | sequence | |
| SEQ ID | SmOPT with mU7- | AAUUUUUGGAGCAGGUUUUCUGCCUUCGGGCGGA |
| NO: 48 | 12C hairpin RNA | AAACCCCU |
| | sequence | |
| SEQ ID | SmOPT-11A with | AATTTTTGGAACAGGGTTTTCTGACTTCGGTCGGAA |
| NO: 49 | mU7-3GG hairpin | AACCCCCT |
| SEQ ID | SmOPT-11A with | AATTTTTGGAACAGGTTTTCTGCCTTCGGGCGGAAA |
| NO: 50 | mU7-12C hairpin | ACCCCT |
| SEQ ID | SmOPT-11A with | AATTTTTGGAACAGGGTTTTCTGCCTTCGGGCGGAA |
| NO: 51 | mU7-3GG-12C | AACCCCCT |
| | hairpin | |
| SEQ ID | SmOPT-11A with | AATTTTTGGAACAGGTTTTCAGACTTCGGTCTGAAA |
| NO: 52 | mU7-10A hairpin | ACCCCT |
| SEQ ID | SmOPT with mU7- | AATTTTTGGAGCAGGGTTTTCAGACTTCGGTCTGAA |
| NO: 53 | 3GG-10A hairpin | AACCCCCT |
| SEQ ID | SmOPT with mU7- | AATTTTTGGAGCAGGGTTTTCTGCCTTCGGGCGGAA |
| NO: 54 | 3GG-12C hairpin | AACCCCCT |
| SEQ ID | SmOPT with mU7- | AATTTTTGGAGCAGGTTTTCAGCCTTCGGGCTGAAA |
| NO: 55 | 10A-12C hairpin | ACCCCT |
| SEQ ID | SmOPT-11A with | AATTTTTGGAACAGGGTTTTCAGACTTCGGTCTGAA |
| NO: 56 | mU7-3GG-10A | AACCCCCT |
| | hairpin | |
| SEQ ID | SmOPT-11A with | AATTTTTGGAACAGGTTTTCAGCCTTCGGGCTGAAA |
| NO: 57 | mU7-10A-12C | ACCCCT |
| | hairpin | |
| SEQ ID | SmOPT with mU7- | AATTTTTGGAGCAGGGTTTTCAGCCTTCGGGCTGA |
| NO: 58 | 3GG-10A-12C | AAACCCCCT |
| | hairpin | |
| SEQ ID | SmOPT-11A with | AATTTTTGGAACAGGGTTTTCAGCCTTCGGGCTGA |
| NO: 59 | mU7-3GG-10A-12C | AAACCCCCT |
| | hairpin | |
| SEQ ID | SmOPT-11A with | AAUUUUUGGAACAGGGUUUUCUGACUUCGGUCGG |
| NO: 60 | mU7-3GG hairpin | AAAACCCCCU |
| | RNA sequence | |
| SEQ ID | SmOPT-11A with | AAUUUUUGGAACAGGUUUUCUGCCUUCGGGCGGA |
| NO: 61 | mU7-12C hairpin | AAACCCCU |
| | RNA sequence | |
| SEQ ID | SmOPT-11A with | AAUUUUUGGAACAGGGUUUUCUGCCUUCGGGCGG |
| NO: 62 | mU7-3GG-12C | AAAACCCCCU |
| | hairpin RNA | |
| | sequence | |
| SEQ ID | SmOPT-11A with | AAUUUUUGGAACAGGUUUUCAGACUUCGGUCUGA |
| NO: 63 | mU7-10A hairpin | AAACCCCU |
| | RNA sequence | |
| SEQ ID | SmOPT with mU7- | AAUUUUUGGAGCAGGGUUUUCAGACUUCGGUCUG |
| NO: 64 | 3GG-10A hairpin | AAAACCCCCU |
| | RNA sequence | |
| SEQ ID | SmOPT with mU7- | AAUUUUUGGAGCAGGGUUUUCUGCCUUCGGGCGG |
| NO: 65 | 3GG-12C hairpin | AAAACCCCCU |
| | RNA sequence | |
| SEQ ID | SmOPT with mU7- | AAUUUUUGGAGCAGGUUUUCAGCCUUCGGGCUGA |
| NO: 66 | 10A-12C hairpin | AAACCCCU |
| | RNA sequence | |
| SEQ ID | SmOPT-11A with | AAUUUUUGGAACAGGGUUUUCAGACUUCGGUCUG |
| NO: 67 | mU7-3GG-10A | AAAACCCCCU |
| | hairpin RNA | |
| | sequence | |
| SEQ ID | SmOPT-11A with | AAUUUUUGGAACAGGUUUUCAGCCUUCGGGCUGA |
| NO: 68 | mU7-10A-12C | AAACCCCU |
| | hairpin RNA | |
| | sequence | |
| SEQ ID | SmOPT with mU7- | AAUUUUUGGAGCAGGGUUUUCAGCCUUCGGGCUG |
| NO: 69 | 3GG-10A-12C | AAAACCCCCU |
| | hairpin RNA | |
| | sequence | |
| SEQ ID | SmOPT-11A with | AAUUUUUGGAACAGGGUUUUCAGCCUUCGGGCUGAAA |
| NO: 70 | mU7-3GG-10A-12C | ACCCCCU |
| | hairpin RNA | |
| | sequence | |
| SEQ ID | 5′ hnRNP A1 domain | TATGATAGGGACTTAGGGTG |
| NO: 71 | | |
| SEQ ID | RAB7A 3′UTR | GTACATAATTCTTGTGTCTACTGTACAGAATACTGCCGC |
| NO: 72 | 80@40 | CAGCTGGATTTCCCAATTCTGAGTAACACTCTGCAATCC |
| | | AA |
| SEQ ID | SNCA 3′UTR 80@40 | GATTGAAGCCACAAAATCCACAGCACACAAAGACCCTG |
| NO: 73 | | CCACCATGTATTCACTTCAGTGAAAGGGAAGCACCGAAA |
| | | TGC |
| SEQ ID | GAPDH 80@40 | GATACCAAAGTTGTCATGGATGACCTTGGCCAGGGGTGC |
| NO: 74 | | CAAGCAGTTGGTGGTGCAGGAGGCATTGCTGATGATCTT |
| | | GA |
| SEQ ID | SOD1 100@50 | GCCCTGCACTGGGCCGTCGCCCTTCAGCACGCACACGGC |
| NO: 75 | | CTTCGTCGCCACAACTCGCTAGGCCACGCCGAGGTCCTG |
| | | GTTCCGAGGACTGCAACGGAAA |
| SEQ ID | FANC 100@50 | GATTGTCCCAAGATGACATCAGCTCATTCTCACAGCCCA |
| NO: 76 | | GCGAGGGCACCTACTCGTGTAATGCGTGGCCACAGGTCA |
| | | TCACCTGTCCTGTGGCCCTGGC |
| SEQ ID | SMAD4 100@50 | AGTCTAAAGGTTGTGCCAGTGCAATCGGCATGGTATGAA |
| NO: 77 | | GTACTTCGTCCAGGAGGACCAGGGCCCGGTGTAAGTGA |
| | | ATTTCAATCCAGCAAGGTGTTTC |
| SEQ ID | DMD exon 71 splice | AAGTACTCACGCAGAATCTACTGGCCAGAAGTTGATCAG |
| NO: 78 | acceptor 100@50 | AGTAACGGGACCGCAAAACAAAAAATGAGGTGGTGAAG |
| | | GAGACACACGCAAACTCAGCCGC |
| SEQ ID | DMD exon 74 splice | CTGGTTCAAACTTTGGCAGTAATGCTGGATTAACAAATG |
| NO: 79 | acceptor 100@50 | TTCATCATCTCCGGAAAATAAAATCAAAGGTTTTGGTTT |
| | | TTTCCCCCCCTTATTTTGCTTT |
| SEQ ID | DMD exon 71 24 mer | AAGTTGATCAGTGTAACGGGACTG |
| NO: 80 | ASO | |
| SEQ ID | DMD exon 74 25 mer | CGAGGCTGGCTCAGGGGGGAGTCCT |
| NO: 81 | ASO | |
| SEQ ID | SmOPT variant | N₀AAUUUUUGN₉GAN₁₁ |
| NO: 114 | consensus sequence | wherein N₀ is absent or U and N₉ or N₁₁ is G, A, C or U |
| | (SmOPT-OU, | |
| | SmOPT-11A, | |
| | SmOPT-11C, and | |
| | SmOPT-11U) | |
| SEQ ID | SmOPT variant | AAUUUUUGN₉AN₁₁ |
| NO: 83 | consensus sequence | wherein N₉ or N₁₁ is A, C or U |
| | (SmOPT-11A, | |
| | SmOPT-11C, and | |
| | SmOPT-11U) | |
| SEQ ID | SmOPT-OU | UAAUUUUUGGAG |
| NO: 84 | | |
| SEQ ID | SmOPT-11C | AAUUUUUGGAC |
| NO: 85 | | |
| SEQ ID | SmOPT-11U | AAUUUUUGGAU |
| NO: 86 | | |
| SEQ ID | SmOPT-11A | AAUUUUUGGAA |
| NO: 87 | | |
| SEQ ID | mU7 variant | CN₂GGN₅N₆N₇UUCN₁₁GN₁₃CUUCGGN₂₀CN₂₂GAAN₂₆N₂₇CCC |
| NO: 88 | consensus sequence | CN₃₂U |
| | | wherein N₂ is A or U, N₅ is either absent or G, |
| | | N₆ is U or A, N₇ is U or C, |
| | | N₁₁ is A or U, N₁₃ is A or C, |
| | | N₂₀ is U or G, N₂₂ U or G, N₂₆ is A or G, |
| | | N₂₇ is A or U, and N₃₂ is either U or absent |
| SEQ ID | mU7 variant | CAGGN₅UUUUCUGN₁₃CUUCGGN₂₀CGGAAAACCCCN₃₂U |
| NO: 89 | consensus sequence | wherein N₅ is either absent or G, N₁₃ is either A or C, |
| | (mU7-3GG, mU7- | N₂₀ is either U or G and N₃₂ is either U or absent |
| | 12C, mU7-3GG-12C) | |
| SEQ ID | mU7-2U hairpin | CUGGUUUUCUGACUUCGGUCGGAAAACCCCU |
| NO: 90 | RNA sequence | |
| SEQ ID | mU7-3GG hairpin | CAGGGUUUUCUGACUUCGGUCGGAAAACCCCCU |
| NO: 91 | RNA sequence | |
| SEQ ID | mU7-5A hairpin | CAGGAUUUCUGACUUCGGUCGGAAAUCCCCU |
| NO: 92 | RNA sequence | |
| SEQ ID | mU7-6C hairpin | CAGGUCUUCUGACUUCGGUCGGAAGACCCCU |
| NO: 93 | RNA sequence | |
| SEQ ID | mU7-10A hairpin | CAGGUUUUCAGACUUCGGUCUGAAAACCCCU |
| NO: 94 | RNA sequence | |
| SEQ ID | mU7-12C hairpin | CAGGUUUUCUGCCUUCGGGCGGAAAACCCCU |
| NO: 95 | RNA sequence | |
| SEQ ID | mU7-3GG-12C | CAGGGUUUUCUGCCUUCGGGCGGAAAACCCCCU |
| NO: 96 | hairpin RNA | |
| | sequence | |
| SEQ ID | mU7-3GG-10A | CAGGGUUUUCAGACUUCGGUCUGAAAACCCCCU |
| NO: 97 | hairpin RNA | |
| | sequence | |
| SEQ ID | mU7-10A-12C | CAGGUUUUCAGCCUUCGGGCUGAAAACCCCU |
| NO: 98 | hairpin RNA | |
| | sequence | |
| SEQ ID | mU7-3GG-10A-12C | CAGGGUUUUCAGCCUUCGGGCUGAAAACCCCCU |
| NO: 99 | hairpin RNA | |
| | sequence | |
| SEQ ID | DMD exon 2 “A” | GTTTTCTTTTGAACATCTTCTCTTTCATCTA |
| NO: 100 | ASO | |
| SEQ ID | DMD exon 2 “C” | ATTCTTACCTTAGAAAATTGTGC |
| NO: 101 | ASO | |
| SEQ ID | DMD exon 2 | CCATTCTTACCTTAGAAAATTGTGCATTTACCCATTTIGT |
| NO: 102 | “A” + “C” ASO | GAATGTTTTCTTTTGAACATCTTCTCTTTCATCTA |
| SEQ ID | DMD exon 51 | GCAGGTACCTCCAACATCAAGGAAGATGGCATTTCTAGT |
| NO: 103 | “long1” ASO | TTGGAG |
| SEQ ID | DMD exon 51 “dt” | CCTCTGTGATTTTATAACTTGAT |
| NO: 104 | ASO | TCAAGGAAGATGGCATTTCT |

### Target RNAs and Mutations

In various embodiments described here, engineered RNAs provided here (engineered guide RNA, ASO) can comprise a targeting sequence having substantial complementarity to a target RNA, and an engineered SmOPT variant sequence, an engineered U7 hairpin variant sequence, or both the engineered SmOPT variant sequence and the engineered U7 hairpin variant sequence, where the engineered guide RNAs of the disclosure are useful as therapeutics for treating subjects suffering from a disease or condition, where the subject can have a target RNA comprising a mutation or a target RNA in need of a mutation. In some examples of the disclosure, a method of treating (including preventing, reducing, or ameliorating) a subject suffering from a disease or a condition or symptoms of the disease or the condition, comprises administering to the subject, a therapeutic that facilitates editing of a target RNA. In some embodiments, editing the target RNA can facilitate correction of a mutation. For example, an engineered RNA of the present disclosure can facilitate an edit of an adenosine present in the mutation to an inosine (read as guanosine). By targeting RNAs with a G to A point mutations, engineered RNAs of the present disclosure can correct the mutation by utilizing ADAR1 or ADAR2-mediated editing of the mutation directly. In some cases, a mutation can be corrected through exon skipping as described herein. An engineered RNA as described herein (engineered guide RNA, ASO) can facilitate an edit of an adenosine via ADAR1 or ADAR2 that facilitates skipping of an exon harboring a particular mutation, thereby restoring functional protein. In some instances, an engineered RNA of the present disclosure (engineered guide RNA, ASO) can be designed or configured to inhibit, cover, mask, or block a target sequence harboring a mutation in a target RNA, thereby promoting exon skipping of the mutation in the target RNA in the absence of ADAR meditated editing. In some instances, an engineered RNA of the present disclosure can facilitate editing a target adenosine and can inhibit, cover, mask, or block a target sequence, both of which inducing exon skipping of a mutation in a target RNA.

The mutation may be a missense mutation or a nonsense mutation. In some embodiments, the engineered guide RNAs of the present disclosure can facilitate multiple RNA edits of a target RNA. By “mutation” as used herein, refers to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” refers to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample is benign. As an alternative, absence of a mutation may not indicate that a tissue or sample is benign. Methods as described herein can comprise identifying a presence of a mutation in a sample.

In some instances, an engineered RNA of the present disclosure (engineered guide RNA, ASO) can be designed or configured to inhibit, cover, mask, or block a target sequence in a target RNA, thereby knocking down expression of protein translated from the target RNA. In some embodiments, an engineered RNA described herein (engineered guide RNA or ASO) can introduce a mutation in order to produce protein knockdown. Protein knockdown can also be referred to as reduced expression of wild-type protein. Engineered guide RNAs of the present disclosure can target one or any combination of a translation initiation site (TIS), an untranslated region such as a 5′ UTR, a polyadenylation (polyA) signal site, or a splice site.

TIS. In some embodiments, the engineered RNAs of the present disclosure target the adenosine at a translation initiation site (TIS). The engineered guide RNAs facilitate ADAR-mediated RNA editing of the TIS (AUG) to GUG. This results in inhibition of RNA translation and, thereby, protein knockdown.

5′UTR. In some embodiments, the engineered guide RNAs of the present disclosure target one or more adenosines in the 5′ untranslated region (5′ UTR). In some embodiments, an engineered guide RNA of the present disclosure can target a Kozak sequence of the 5′ UTR. In some embodiments, an engineered guide RNA of the present disclosure can target an internal ribosomal entry site (IRES) of the 5′ UTR. In some embodiments, an engineered guide RNA of the present disclosure can target an iron response element (IRE) of the 5′ UTR. In some embodiments, an engineered guide RNA facilitates ADAR-mediated RNA editing of one or more adenosines the 5′UTR (including one or more adenosines present in one or more structures of the 5′ UTR). In some instances, extensive or hyper editing of a plurality of adenosines can be facilitated via an engineered guide RNA of the present disclosure, which can result in ribosomal stalling of the mRNA transcript, thereby resulting in protein knockdown.

Splice site. In some embodiments, the engineered RNAs of the present disclosure target an adenosine at a splice site. The engineered guide RNAs facilitate ADAR-mediated RNA editing of an A at a splice site. This can result in mistranslation and/or truncation of a protein encoded by the pre-mRNA molecule and, thereby, protein knockdown.

PolyA Signal Sequence. In some embodiments, the engineered RNAs of the present disclosure target one or more adenosines in the polyA signal sequence. In some embodiments, an engineered guide RNA facilitates ADAR-mediated RNA editing of the one or more adenosines in the polyA signal sequence, thereby resulting in disruption of RNA processing and degradation of the target mRNA and, thereby, protein knockdown. In some embodiments, a target can have one or more polyA signal sequences. In these instances, one or more engineered RNAs, varying in their respective sequences, of the present disclosure can be multiplexed to target adenosines in the one or more polyA signal sequences. In both cases, the engineered RNAs of the present disclosure facilitated ADAR-mediated RNA editing of adenosines to inosines (read as guanosines by cellular machinery) in the polyA signal sequence, resulting in protein knockdown.

ABCA4. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets ABCA4 RNA. In particular cases, an engineered RNA payload such as an ASO or guide RNA targeting ABCA4, is operably linked to any one of SEQ ID NO: 49 (RNA sequence of SEQ ID NO: 60). In some cases, an engineered RNA payload such as an ASO or guide RNA targeting ABCA4, is operably linked to any one of SEQ ID NO: 50 (RNA sequence of SEQ ID NO: 61). In particular cases, an engineered RNA payload such as an ASO or guide RNA targeting ABCA4, is operably linked to any one of SEQ ID NO: 51 (RNA sequence of SEQ ID NO: 62). Some examples provide the engineered RNAs (e.g., engineered guide RNA, ASO) of the disclosure, which can comprise a targeting sequence with target complementarity to a target RNA that is an ATP binding cassette subfamily A member 4 (ABCA4). In some embodiments of the disclosure, the engineered guide RNAs or therapeutics described here, comprising an engineered SmOPT variant sequence, an engineered U7 hairpin variant sequence, or both the engineered SmOPT variant sequence and the engineered U7 hairpin variant sequence, can facilitate RNA editing of an ABCA4 target RNA, which can have a mutation selected from the group consisting of: G6320A; G5714A; G5882A; and any combination thereof. The engineered guide RNAs described here comprising the engineered SmOPT variant sequence, the engineered U7 hairpin variant sequence, or both the engineered SmOPT variant sequence and the engineered U7 hairpin variant sequence among other RNA elements, can correct the G to A mutations of the ABCA4 gene. The engineered guide RNAs of the disclosure can comprise an engineered SmOPT variant sequence having up to 90.9% sequence identity to SEQ ID NO: 1 or SEQ ID NO: 2; an engineered U7 hairpin variant sequence having up to 96.8% sequence identity to SEQ ID NO: 3 or having up to 96.9% sequence identity to SEQ ID NO: 4, or both the engineered SmOPT variant sequence and the engineered U7 hairpin variant sequence. In other embodiments, the engineered guide RNAs described here can comprise an engineered SmOPT variant sequence having at least one polynucleotide substitution (e.g., 1, 2, 3, 4, 5, 6, 7, 8, 9, 10, 11) as compared to SEQ ID NO: 1 or SEQ ID NO: 2. Some embodiments provide the engineered guide RNAs described here, where the engineered guide RNAs comprise an engineered U7 hairpin variant sequence having at least one polynucleotide substitution (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32) as compared to SEQ ID NO: 3 or SEQ ID NO: 4. Further embodiments can provide engineered guide RNAs comprising a targeting sequence having substantial complementarity to an ABCA4 target, and an engineered SmOPT variant sequence, an engineered U7 hairpin variant sequence, or both the engineered SmOPT variant sequence and the engineered U7 hairpin variant sequence, incorporated with any promoter (e.g., U1, U6, or U7) disclosed here, which can drive expression of the engineered guide RNAs. In some examples, the ABCA4 mutation causes or contributes to macular degeneration in a subject in need thereof to whom the described engineered guide RNA can be administered for treatment. In some examples, the macular degeneration can be Stargardt macular degeneration. In some embodiments, the human subject can be at risk of developing or has developed Stargardt macular degeneration (or Stargardt's disease), which could be caused, at least in part, by one of the indicated mutations of ABCA4. Some embodiments of the disclosure provide for the engineered guide RNAs comprising a targeting sequence having substantial complementarity to an ABCA4 target RNA, and an engineered SmOPT variant sequence, an engineered U7 hairpin variant sequence, or both the engineered SmOPT variant sequence and the engineered U7 hairpin variant sequence, for facilitating editing thereby correcting the mutation in ABCA4 and reducing the incidence of Stargardt's disease in the subject. In some examples the target RNA molecule comprises an adenosine with a 5′G. In some examples, the adenosine with the 5′G can be the base intended for chemical modification by the RNA editing entity. In some examples, the RNA editing entity can be an ADAR, and the ADAR chemically modifies the adenosine with the 5′G after recruitment by the guide-target RNA scaffold. Accordingly, such engineered guide RNAs can be used in methods of treating a subject suffering from Stargardt macular degeneration.

APP. Other examples of the disclosure can be directed to engineered guide RNAs, where the target RNA is amyloid precursor protein (APP), which can be targeted for editing. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets APP RNA. In particular cases, an engineered RNA payload such as an ASO or guide RNA targeting APP, is operably linked to any one of SEQ ID NO: 49 (RNA sequence of SEQ ID NO: 60). In some cases, an engineered RNA payload such as an ASO or guide RNA targeting APP, is operably linked to any one of SEQ ID NO: 50 (RNA sequence of SEQ ID NO: 61). In particular cases, an engineered RNA payload such as an ASO or guide RNA targeting APP, is operably linked to any one of SEQ ID NO: 51 (RNA sequence of SEQ ID NO: 62). In some embodiments, a specific residue can be targeted utilizing the engineered RNAs (e.g., engineered guide RNA, ASO) comprising an engineered SmOPT variant sequence, an engineered U7 hairpin variant sequence, or both the engineered SmOPT variant sequence and the engineered U7 hairpin variant sequence and methods described here. In some examples, the engineered RNAs described here (e.g., engineered guide RNA, ASO) are configured to facilitate an edit of a base of a nucleotide of the target RNA by an RNA editing entity forming an edited target RNA, such that a protein translated from the edited target RNA comprises at least one alteration or mutation selected from the group consisting of: K670E, K670R, K670G, M671V, A673V, A673T, D672G, E682G, H684R, K687R, K687E, K687G, I712X, T714X, and any combination thereof. In some embodiments of the disclosure, the target RNA encodes for an unmodified APP polypeptide that comprises at least one amino acid residue difference as compared to a modified APP polypeptide generated from editing a base of a nucleotide of the APP target RNA, where the at least one amino acid residue difference is selected from the group consisting of: K670E, K670R, K670G, M671V, A673V, A673T, D672G, E682G, H684R, K687R, K687E, K687G, I712X, T714X of the APP polypeptide, and any combination thereof. In some examples, the target RNA molecule encodes, at least in part, an amyloid precursor protein (APP); an APP start site; an APP cleavage site; or a beta secretase (BACE) or gamma secretase cleavage site of an APP protein. In some examples, cleavage of the APP protein at the cleavage site causes or contributes to Amyloid beta (Aβ or Abeta) peptide deposition in the brain or blood vessels. In some examples, the Abeta deposition causes or contributes to a neurodegenerative disease. In some examples, the disease comprises Alzheimer's disease, Parkinson's disease, corticobasal degeneration, dementia with Lewy bodies, Lewy body variant of Alzheimer's disease, Parkinson's disease with dementia, Pick's disease, progressive supranuclear palsy, dementia, fronto-temporal dementia with Parkinsonism linked to tau mutations on chromosome 17, or any combination thereof. The engineered RNAs of the disclosure (e.g., engineered guide RNA, ASO) comprising a targeting sequence having substantial complementarity to an APP target RNA and comprising an engineered SmOPT variant sequence, an engineered U7 hairpin variant sequence, or both the engineered SmOPT variant sequence and the engineered U7 hairpin variant sequence can be used to facilitate an edit of a base of a nucleotide of the APP target RNA by an RNA editing entity forming an edited APP target RNA, such that a protein translated from the edited APP target RNA comprises at least one alteration or mutation described here. Accordingly, such engineered RNAs can be used in methods of treating a subject suffering from a neurodegenerative disease, such as but not limited to, Alzheimer's disease, Parkinson's disease, dementia, and the like.

DMPK. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets DMPK RNA. In particular cases, an engineered RNA payload such as an ASO or guide RNA targeting DMPK, is operably linked to any one of SEQ ID NO: 49 (RNA sequence of SEQ ID NO: 60). In some cases, an engineered RNA payload such as an ASO or guide RNA targeting DMPK, is operably linked to any one of SEQ ID NO: 50 (RNA sequence of SEQ ID NO: 61). In particular cases, an engineered RNA payload such as an ASO or guide RNA targeting DMPK, is operably linked to any one of SEQ ID NO: 51 (RNA sequence of SEQ ID NO: 62). In some embodiments, the present disclosure provides engineered RNAs (e.g., engineered guide RNA, ASO) comprising a targeting sequence sufficiently complementary to a DMPK target RNA, and an engineered SmOPT variant sequence, an engineered U7 hairpin variant sequence, or both the engineered SmOPT variant sequence and the engineered U7 hairpin variant sequence, that facilitate RNA editing of DMPK to knockdown expression of myotonic dystrophy protein kinase. In some instances, an engineered RNA of the present disclosure (engineered guide RNA, ASO) can be designed or configured to inhibit, cover, mask, or block a target sequence in a target DMPK RNA, thereby knocking down expression of myotonic dystrophy protein kinase. Myotonic dystrophy (DM1) is a rare neuromuscular disease characterized by progressive muscular weakness and an inability to relax muscles (myotonia), predominantly distal skeletal muscles. In some embodiments, the engineered RNAs of the present disclosure (e.g., engineered guide RNA, ASO) comprising an engineered SmOPT variant sequence, an engineered U7 hairpin variant sequence, or both the engineered SmOPT variant sequence and the engineered U7 hairpin variant sequence target DMPK and compositions comprising such engineered guide RNAs facilitate ADAR-mediated RNA editing of DMPK to knockdown expression of myotonic dystrophy protein kinase.

DUX4. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets DUX4 RNA. In particular cases, an engineered RNA payload such as an ASO or guide RNA targeting DUX4, is operably linked to any one of SEQ ID NO: 49 (RNA sequence of SEQ ID NO: 60). In some cases, an engineered RNA payload such as an ASO or guide RNA targeting DUX4, is operably linked to any one of SEQ ID NO: 50 (RNA sequence of SEQ ID NO: 61). In particular cases, an engineered RNA payload such as an ASO or guide RNA targeting DUX4, is operably linked to any one of SEQ ID NO: 51 (RNA sequence of SEQ ID NO: 62). The present disclosure provides engineered RNAs (e.g., engineered guide RNA, ASO) comprising an engineered SmOPT variant sequence an engineered U7 hairpin variant sequence, or both the engineered SmOPT variant sequence and the engineered U7 hairpin variant sequence, and a targeting sequence sufficiently complementary to a DUX4 target RNA that facilitate RNA editing DUX4 to knockdown expression of DUX4 protein. In some instances, an engineered RNA of the present disclosure (engineered guide RNA, ASO) can be designed or configured to inhibit, cover, mask, or block a target sequence in a target DUX4 RNA, thereby knocking down expression of DUX4 protein. Facioscapulohumeral muscular dystrophy (FSHD), an autosomal dominant neuromuscular disorder, is a rare neuromuscular disease characterized by progressive skeletal muscle weakness with significant heterogeneity in phenotypic severity and age of onset. Genetic causes of FSHD include mutations in the D4Z4 repeat region on chromosome 4 that lead to hypomethylation and dysregulated expression of the DUX4 gene (a germline transcription factor). In some embodiments, the present disclosure provides compositions of engineered RNAs (e.g., engineered guide RNA, ASO) that target DUX4 and comprise an engineered SmOPT variant sequence, an engineered U7 hairpin variant sequence, or both the engineered SmOPT variant sequence and the engineered U7 hairpin variant sequence, and facilitate ADAR-mediated RNA editing of DUX4, specifically, DUX4-FL to mediate DUX4-FL knockdown.

In some embodiments, the engineered RNAs of the present disclosure (e.g., engineered guide RNA, ASO) facilitate ADAR-mediated RNA editing of target genes (e.g., DMPK, DUX4-FL), which results in knockdown of protein levels. In some instances, an engineered RNA of the present disclosure (engineered guide RNA, ASO) can be designed or configured to inhibit, cover, mask, or block a target sequence in a target RNA, thereby knocking down expression of the protein translated from the target RNA. The knockdown in protein levels is quantitated as a reduction in expression of the protein (e.g., DMPK protein: myotonic dystrophy protein kinase; DUX4-FL protein). The engineered RNAs of the present disclosure (e.g., engineered guide RNA, ASO) comprising a targeting sequence sufficiently complementary to, for example, a DMPK or DUX4 target of interest, and an engineered SmOPT variant sequence, an engineered U7 hairpin variant sequence, or both the engineered SmOPT variant sequence and the engineered U7 hairpin variant sequence can facilitate from 1% to 100% DMPK protein knockdown or DUX4-FL protein knockdown. The engineered RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% DMPK protein knockdown or DUX4-FL protein knockdown. In some embodiments, the engineered RNAs of the present disclosure (e.g., engineered guide RNA, ASO) facilitate from 30% to 60% DMPK protein knockdown or DUX4-FL protein knockdown. DMPK protein knockdown or DUX4-FL protein knockdown can be measured by an assay comparing a sample or subject treated with the engineered RNA of the present disclosure (e.g., engineered guide RNA, ASO) comprising an engineered SmOPT variant sequence, an engineered U7 hairpin variant sequence, or both the engineered SmOPT variant sequence and the engineered U7 hairpin variant sequence to a control sample or subject not treated with the engineered guide RNA comprising an engineered SmOPT variant sequence an engineered U7 hairpin variant sequence, or both the engineered SmOPT variant sequence and the engineered U7 hairpin variant sequence.

DMD. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets DMD RNA. In particular cases, an engineered RNA payload such as an ASO or guide RNA targeting DMD, is operably linked to any one of SEQ ID NO: 49 (RNA sequence of SEQ ID NO: 60). In some cases, an engineered RNA payload such as an ASO or guide RNA targeting DMD, is operably linked to any one of SEQ ID NO: 50 (RNA sequence of SEQ ID NO: 61). In particular cases, an engineered RNA payload such as an ASO or guide RNA targeting DMD, is operably linked to any one of SEQ ID NO: 51 (RNA sequence of SEQ ID NO: 62). In some embodiments, the present disclosure provides engineered RNAs (e.g., engineered guide RNA, ASO) comprising a targeting sequence sufficiently complementary to a DMD target RNA, and an engineered SmOPT variant sequence, an engineered U7 hairpin variant sequence, or both the engineered SmOPT variant sequence and the engineered U7 hairpin variant sequence, that facilitate exon skipping of a DMD pre-RNA in order to produce functional dystrophin protein. Duchenne Muscular Dystrophy (DMD) is a rare neuromuscular disease typically characterized by a loss of one or more exons of the dystrophin protein. Progression of DMD results in a weakening of muscles over time in an irreversible manner. In some embodiments, an engineered RNA of the present disclosure (engineered guide RNA, ASO) comprising an engineered SmOPT variant sequence, an engineered U7 hairpin variant sequence, or both the engineered SmOPT variant sequence and the engineered U7 hairpin variant sequence target DMPK and compositions comprising such engineered guide RNAs can restore functional dystrophin protein from a DMD transcript harboring a mutation that results in the progression of DMD by facilitating skipping of an exon harboring the mutation. In some cases, the engineered RNA can induce the skipping of the exon 2 in the DMD pre-RNA in the subject. In some cases, the engineered RNA can induce the skipping of the exon 51 DMD pre-RNA in the subject. In some cases, the engineered RNA can induce the skipping of the exon 45 in the DMD pre-RNA in the subject. In some cases, the engineered RNA can induce the skipping of the exon 53 in the DMD pre-RNA in the subject. In some cases, the engineered RNA can induce the skipping of the exon 44 in the DMD pre-RNA in the subject. In some cases, the engineered RNA can induce the skipping of the exon 52 in the DMD pre-RNA in the subject. In some cases, the engineered RNA can induce the skipping of the exon 50 in the DMD pre-RNA in the subject. In some cases, the engineered RNA can induce the skipping of the exon 71 in the DMD pre-RNA in the subject. In some cases, the engineered RNA can induce the skipping of the exon 74 in the DMD pre-RNA in the subject.

LRRK2. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets LRRK2 RNA. In particular cases, an engineered RNA payload such as an ASO or guide RNA targeting LRRK2, is operably linked to any one of SEQ ID NO: 49 (RNA sequence of SEQ ID NO: 60). In some cases, an engineered RNA payload such as an ASO or guide RNA targeting LRRK2, is operably linked to any one of SEQ ID NO: 50 (RNA sequence of SEQ ID NO: 61). In particular cases, an engineered RNA payload such as an ASO or guide RNA targeting LRRK2, is operably linked to any one of SEQ ID NO: 51 (RNA sequence of SEQ ID NO: 62). The described engineered RNAs of the disclosure (e.g., engineered guide RNA, ASO) can comprise a targeting sequence with target complementarity to a leucine-rich repeat kinase 2 (LRRK2) target RNA, further comprising an engineered SmOPT variant sequence, an engineered U7 hairpin variant sequence, or both the engineered SmOPT variant sequence and the engineered U7 hairpin variant sequence. In some embodiments, such engineered RNAs can facilitate RNA editing of LRRK2 encoded mutations associated with a disease or condition, where a LRRK2 encoded mutation can be selected from the group consisting of: E10L, A30P, S52F, E46K, A53T, L119P, A211V, C₂₂₈S, E334K, N363S, V366 M, A419V, R506Q, N544E, N551K, A716V, M712V, I723V, P755L, R793 M, I810V, K871E, Q923H, Q930R, R1067Q, S1096C, Q1111H, I1122V, A1151T, L1165P, 11192V, H1216R, S1228T, P1262A, R1325Q, I1371V, R1398H, T1410 M, D1420N, N1437H, R1441C, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141 M, R2143H, Y2189C, T2356I, G2385R, V2390 M, E2395K, M2397T, L2466H, Q2490N, and any combination thereof. In some embodiments, such engineered RNAs that target LRRK2 can be used for treating a disease or condition such as a neurodegenerative disease (Parkinson's) by producing an edit, a knockdown or both of a pathogenic variant of LRRK2. In some aspects, a pathogenic variant of LRRK2 can comprise a G2019S mutation. The engineered RNAs targeting LRRK2 (e.g., engineered guide RNA, ASO) and comprising an engineered SmOPT variant sequence, an engineered U7 hairpin variant sequence, or both the engineered SmOPT variant sequence and the engineered U7 hairpin variant sequence can be used to treat a LRRK2 associated disease or condition such as but not limited to a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof.

MAPT. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets MAPT RNA. In particular cases, an engineered RNA payload such as an ASO or guide RNA targeting MAPT, is operably linked to any one of SEQ ID NO: 49 (RNA sequence of SEQ ID NO: 60). In some cases, an engineered RNA payload such as an ASO or guide RNA targeting MAPT, is operably linked to any one of SEQ ID NO: 50 (RNA sequence of SEQ ID NO: 61). In particular cases, an engineered RNA payload such as an ASO or guide RNA targeting MAPT, is operably linked to any one of SEQ ID NO: 51 (RNA sequence of SEQ ID NO: 62). In some embodiments, the engineered RNA of the present disclosure (engineered guide RNA, ASO) comprising an engineered SmOPT variant sequence, an engineered U7 hairpin variant sequence, or both target a coding sequence of an RNA (e.g., a TIS such as the c.1 TIS, the c.31 TIS, the c.91 TIS, or the c.379 TIS of MAPT). In some embodiments, the engineered RNA of the present disclosure (engineered guide RNA, ASO) comprising an engineered SmOPT variant sequence, an engineered U7 hairpin variant sequence, or both target a non-coding sequence of an RNA (e.g., a polyA sequence). The present disclosure provides engineered RNAs (e.g., engineered guide RNA, ASO) comprising an engineered SmOPT variant sequence an engineered U7 hairpin variant sequence, or both the engineered SmOPT variant sequence and the engineered U7 hairpin variant sequence, and a targeting sequence sufficiently complementary to a MAPT target RNA that facilitate RNA editing MAPT to knockdown expression of Tau protein. In some instances, an engineered RNA of the present disclosure (engineered guide RNA, ASO) can be designed or configured to inhibit, cover, mask, or block a target sequence in a target MAPT RNA, thereby knocking down expression of Tau protein. Tau pathology can be a key driver of a broad spectrum of neurodegenerative diseases, collectively known as Tauopathies. For example, diseases where Tau can play a primary role include, but are not limited to, Alzheimer's disease (AD), frontotemporal dementia (FTD), Parkinson's disease, progressive supranuclear palsy (PSP), corticobasal degeneration (CBD), and chronic traumatic encephalopathy. Tauopathies are characterized by the intracellular accumulation of neurofibrillary tangles (NFTs) composed of aggregated, misfolded Tau (MAPT gene). Thus, engineered RNAs of the present disclosure (engineered guide RNA, ASO) comprising an engineered SmOPT variant sequence, an engineered U7 hairpin variant sequence, or both and targeting MAPT RNA for ADAR-mediated editing to knockdown Tau protein can be capable of preventing or ameliorating disease progression in a number of diseases, including, but not limited to, AD, FTD, autism, traumatic brain injury, Parkinson's disease, and Dravet syndrome.

PMP22. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets PMP22 RNA. In particular cases, an engineered RNA payload such as an ASO or guide RNA targeting PMP22, is operably linked to any one of SEQ ID NO: 49 (RNA sequence of SEQ ID NO: 60). In some cases, an engineered RNA payload such as an ASO or guide RNA targeting PMP22, is operably linked to any one of SEQ ID NO: 50 (RNA sequence of SEQ ID NO: 61). In particular cases, an engineered RNA payload such as an ASO or guide RNA targeting PMP22, is operably linked to any one of SEQ ID NO: 51 (RNA sequence of SEQ ID NO: 62). The present disclosure provides for engineered RNAs (e.g., engineered guide RNA, ASO) targeting PMP22 and comprising an engineered SmOPT variant sequence, an engineered U7 hairpin variant sequence, or both the engineered SmOPT variant sequence and the engineered U7 hairpin variant sequence that facilitate RNA editing of PMP22 to knockdown expression of peripheral myelin protein-22 (PMP22). In some instances, an engineered RNA of the present disclosure (engineered guide RNA, ASO) can be designed or configured to inhibit, cover, mask, or block a target sequence in a target PMP22 RNA, thereby knocking down expression of PMP22 protein. Charcot-Marie-Tooth Syndrome (CMT1A) is the most common genetically-driven peripheral neuropathy, characterized by progressive distal muscle atrophy, sensory loss and foot/hand deformities. In some embodiments, the present disclosure provides compositions of engineered RNAs (e.g., engineered guide RNA, ASO) that target PMP22 and comprise an engineered SmOPT variant sequence, an engineered U7 hairpin variant sequence, or both the engineered SmOPT variant sequence and the engineered U7 hairpin variant sequence, which facilitate ADAR-mediated RNA editing of PMP22. In some embodiments, the engineered RNAs of the present disclosure (e.g., engineered guide RNA, ASO) target a coding sequence in PMP22. For example, the coding sequence can be a translation initiation site (TIS) (AUG) of PMP22 and the engineered RNA can facilitate ADAR-mediated RNA editing of AUG to GUG. The engineered RNAs of the present disclosure (e.g., engineered guide RNA, ASO) that target PMP22 and comprise an engineered SmOPT variant sequence, an engineered U7 hairpin variant sequence, or both the engineered SmOPT variant sequence and the engineered U7 hairpin variant sequence can facilitate ADAR-mediated RNA editing of PMP22, thereby, effecting its protein knockdown.

SERPINA1. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets SERPINA1 RNA. In particular cases, an engineered RNA payload such as an ASO or guide RNA targeting SERPINA1, is operably linked to any one of SEQ ID NO: 49 (RNA sequence of SEQ ID NO: 60). In some cases, an engineered RNA payload such as an ASO or guide RNA targeting SERPINA1, is operably linked to any one of SEQ ID NO: 50 (RNA sequence of SEQ ID NO: 61). In particular cases, an engineered RNA payload such as an ASO or guide RNA targeting SERPINA1, is operably linked to any one of SEQ ID NO: 51 (RNA sequence of SEQ ID NO: 62). In some embodiments, the disclosure is directed to an engineered RNA (e.g., engineered guide RNA, ASO) comprising a targeting sequence substantially complementary to the serpin family A member 1 (SERPINA1) target RNA, and an engineered SmOPT variant sequence, an engineered U7 hairpin variant sequence, or both the engineered SmOPT variant sequence and the engineered U7 hairpin variant sequence, where the engineered RNA can facilitate RNA editing of SERPINA1. For example, such engineered RNAs can correct a G to A mutation at nucleotide position 9989 of a SERPINA1 gene (G9989A) or the SERPINA1 target RNA encodes a mutation of E342K. In some examples, the mutation causes or contributes to an antitrypsin (AAT) deficiency, such as alpha-1 antitrypsin deficiency (AATD) in a subject to whom the engineered guide RNA of the disclosure can be administered. Some embodiments are directed to methods of treating a subject who can be human and at risk of developing or has developed alpha-1 antitrypsin deficiency. Such alpha-1 antitrypsin deficiency can be at least partially caused by a mutation of SERPINA1, for which an engineered RNA (e.g., engineered guide RNA, ASO) comprising an engineered SmOPT variant sequence, an engineered U7 hairpin variant sequence, or both the engineered SmOPT variant sequence and the engineered U7 hairpin variant sequence described here can facilitate editing of the mutation in, for example, a human subject, thus correcting the mutation in SERPINA1 and reducing the incidence of alpha-1 antitrypsin deficiency in the subject. Accordingly, the engineered RNAs of the present disclosure (e.g., engineered guide RNA, ASO) targeting SERPINA1 and having an engineered SmOPT variant sequence, an engineered U7 hairpin variant sequence, or both the engineered SmOPT variant sequence and the engineered U7 hairpin variant sequence can be used in a method of treating a subject suffering from an alpha-1 antitrypsin deficiency.

Some aspects provide engineered RNAs (e.g., engineered guide RNA, ASO) comprising exemplary targeting sequences that can target a SERPINA1 gene linked to any promoter (e.g., U1, U6, U7) disclosed herein that can be incorporated to drive expression of the engineered guide RNAs. Alpha-1 antitrypsin deficiency can be at least partially caused by a mutation of SERPINA1, for which the engineered RNA described herein (e.g., engineered guide RNA, ASO) can facilitate editing in, thus correcting the mutation in SERPINA1 and reducing the incidence of alpha-1 antitrypsin deficiency in the subject.

SNCA. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets SNCA RNA. In particular cases, an engineered RNA payload such as an ASO or guide RNA targeting SNCA, is operably linked to any one of SEQ ID NO: 49 (RNA sequence of SEQ ID NO: 60). In some cases, an engineered RNA payload such as an ASO or guide RNA targeting SNCA, is operably linked to any one of SEQ ID NO: 50 (RNA sequence of SEQ ID NO: 61). In particular cases, an engineered RNA payload such as an ASO or guide RNA targeting SNCA, is operably linked to any one of SEQ ID NO: 51 (RNA sequence of SEQ ID NO: 62). In some embodiments, the present disclosure provides engineered RNAs (e.g., engineered guide RNA, ASO), compositions, and methods of using the engineered RNAs (e.g., engineered guide RNA, ASO) comprising engineered SmOPT variant sequences, engineered U7 hairpin variant sequences, or both the engineered SmOPT variant sequence and the engineered U7 hairpin variant sequence that can facilitate RNA editing of SNCA. In some embodiments, such engineered RNAs described herein (e.g., engineered guide RNA, ASO) having engineered SmOPT variant sequences, engineered U7 hairpin variant sequences, or both the engineered SmOPT variant sequence and the engineered U7 hairpin variant sequence can knock down expression of SNCA, for example, by facilitating editing at a 3′ UTR of an SNCA RNA. In some instances, an engineered RNA of the present disclosure (engineered guide RNA, ASO) can be designed or configured to inhibit, cover, mask, or block a target sequence in a target SNCA RNA, thereby knocking down expression of SNCA protein. Such engineered RNAs comprising engineered SmOPT variant sequences and engineered U7 hairpin variant sequences targeting a site in SNCA can be encoded for by an engineered polynucleotide construct of the present disclosure. In some embodiments of the disclosure, the engineered RNAs described here (e.g., engineered guide RNA, ASO) comprising a targeting sequence with target complementarity to an alpha-synuclein (SNCA) target RNA, and an engineered SmOPT variant sequence, an engineered U7 hairpin variant sequence, or both the engineered SmOPT variant sequence and the engineered U7 hairpin variant sequence, can be used in the treatment of neurodegenerative disease, including but not limited to Alzheimer's disease or other diseases associated with an accumulation of Tau-p. In some examples, the engineered RNA comprises an engineered guide RNA, which targets (a) an SNCA start codon that can be expressed using a mouse U7 promoter or targets an SNCA 3′UTR expressed using a mouse U7 promoter, with (b) an engineered SmOPT variant sequence, (c) an engineered U7 hairpin variant sequence, or both the engineered SmOPT variant sequence of (b) and the engineered U7 hairpin variant sequence of (c), and operably linked to a mouse U7 terminator sequence or a human U7 terminator sequence. In some embodiments, the mouse U7 terminator or the human U7 terminator is a truncated terminator.

In some examples, polymorphisms in either LRRK2 (G2019S) or SNCA can be associated with an increased risk of idiopathic Parkinson's Disease, and the disease or condition can comprise idiopathic Parkinson's Disease. In some examples, administration of the engineered RNAs disclosed herein can edit LRRK2 G2019S (G>A conversion at the 6055^{th} nucleotide) and edit the start codon SNCA by editing any of the nucleotides of the ATG to decrease the expression of SNCA. Some examples of the disclosure provide for engineered RNAs (e.g., engineered guide RNA, ASO) comprising a targeting sequence with sufficient complementarity to an SNCA target RNA and further comprising an engineered SmOPT variant sequence, an engineered U7 hairpin variant sequence, or both the engineered SmOPT variant sequence and the engineered U7 hairpin variant sequence, where the SNCA comprises a mutation for RNA editing selected from the group consisting of: a translation initiation site (TIS) AUG to GTG in Codon 1, a TIS AUG in Codon 5, an AUG at position 265 in Exon 2, and any combination thereof.

SOD1. Provided herein are engineered RNA payloads, such as engineered guide RNAs or antisense oligonucleotides (ASOs), operably linked to any of or any combination of engineered SmOPT variant sequences or engineered U7 variant sequences disclosed herein, where the engineered RNA payload targets SOD1 RNA. In particular cases, an engineered RNA payload such as an ASO or guide RNA targeting SOD1, is operably linked to any one of SEQ ID NO: 49 (RNA sequence of SEQ ID NO: 60). In some cases, an engineered RNA payload such as an ASO or guide RNA targeting SOD1, is operably linked to any one of SEQ ID NO: 50 (RNA sequence of SEQ ID NO: 61). In particular cases, an engineered RNA payload such as an ASO or guide RNA targeting SOD1, is operably linked to any one of SEQ ID NO: 51 (RNA sequence of SEQ ID NO: 62). The present disclosure provides for engineered RNAs (e.g., engineered guide RNA, ASO) that facilitate RNA editing of SOD1 to knockdown expression of the superoxide dismutase enzyme. In some instances, an engineered RNA of the present disclosure (engineered guide RNA, ASO) can be designed or configured to inhibit, cover, mask, or block a target sequence in a target SOD1 RNA, thereby knocking down expression of SOD1 protein. Amyotrophic lateral sclerosis (ALS) is a rapidly progressing neurodegenerative disease characterized by death of motor neurons and loss of voluntary muscle movement. While the exact cause of ALS is unknown, gain-of-function mutations in SOD1 account for ˜20% of familiar ALS and 2% of spontaneous ALS. In some embodiments, the present disclosure provides compositions of engineered RNAs (e.g., engineered guide RNA, ASO) that target SOD1 and comprise an engineered SmOPT variant sequence, an engineered U7 hairpin variant sequence, or both the engineered SmOPT variant sequence and the engineered U7 hairpin variant sequence, and facilitate ADAR-mediated RNA editing of SOD1. The engineered RNAs of the present disclosure (e.g., engineered guide RNA, ASO) targeting SOD1 and comprising an engineered SmOPT variant sequence, an engineered U7 hairpin variant sequence, or both the engineered SmOPT variant sequence and the engineered U7 hairpin variant sequence facilitate ADAR-mediated RNA editing of SOD1, thereby, effecting protein knockdown.

In some embodiments, an engineered RNA of the disclosure (e.g., engineered guide RNA, ASO) comprising an engineered guide RNA, which targets (a) an SOD1 start codon that can be expressed using, for example, a U6 promoter (human), U7 promoter (mouse), or mouse U7 promoter with a 5′ double hnRNP A1(heterogeneous ribonucleoprotein A1) binding site, with (b) an engineered SmOPT variant sequence, (c) an engineered U7 hairpin variant sequence, or both the engineered SmOPT variant sequence of (b) and the engineered U7 hairpin variant sequence of (c), and operably linked to a terminator sequence, for example, a mouse U7 terminator sequence or human U7 terminator sequence. In some embodiments, the mouse U7 or human U7 terminator is a truncated terminator.

### Delivery Vehicles

Other embodiments of the disclosure can provide for a delivery vehicle comprising any of the engineered RNAs (e.g., engineered guide RNAs, ASOs) of the disclosure comprising a targeting sequence having substantial complementarity to a target RNA and an engineered SmOPT variant sequence, an engineered U7 hairpin variant sequence, or both the engineered SmOPT variant sequence and the engineered U7 hairpin variant sequence, or a polynucleotide encoding any of the engineered RNAs (e.g., engineered guide RNAs, ASOs) of the disclosure. Non-limiting examples of useful delivery vehicles include: a vector, a liposome, a particle, a dendrimer, and any combination thereof. An engineered RNA (e.g., engineered guide RNA, ASO) of the present disclosure or polynucleotide encoding such engineered RNA of the disclosure can be delivered to a subject or at least one cell of a subject via a delivery vehicle. In some embodiments, the delivery vehicle is a vector. A vector can facilitate delivery of an engineered guide RNA comprising an engineered SmOPT variant sequence, an engineered U7 hairpin variant sequence, or both the engineered SmOPT variant sequence and the engineered U7 hairpin variant sequence into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered RNA (e.g., engineered guide RNA, ASO) or polynucleotide encoding the engineered guide RNA of the disclosure can be used to deliver the engineered RNA (e.g., engineered guide RNA, ASO) to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or any combination thereof. In some embodiments, the viral vector can be a recombinant vector, a hybrid vector, a chimeric vector, a self-complementary vector, a single-stranded vector, or any combination thereof.

Some embodiments can provide for a viral vector delivery vehicle, where the viral vector can be an adeno-associated viral (AAV) vector or derivative thereof, where the AAV vector, derivative thereof, or a hybrid of the AAV vector or derivative thereof, can be selected from a group of viral vector serotypes consisting of: AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAVhu68, a derivative of any of these, and a hybrid of any of these. In some embodiments, the AAV vector or derivative thereof can be selected from a group consisting of: a recombinant AAV (rAAV) vector, a hybrid AAV vector, a chimeric AAV vector, a self-complementary AAV (scAAV) vector, and any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered RNA sequences, etc. In some examples, the viral vectors described herein can be engineered through synthetic or other suitable means by references to published sequences, such as those that may be available in the literature. For example, the genomic and protein sequences of various serotypes of AAV, as well as the sequences of the native terminal repeats (TRs), Rep proteins, and capsid subunits can be found in the literature or in public databases such as GenBank or Protein Data Bank (PDB).

In some examples, methods of producing delivery vectors herein comprising packaging an engineered RNA of the present disclosure (e.g., a polynucleotide encoding for an engineered RNA) in an AAV vector. In some examples, methods of producing the delivery vectors described herein comprise, (a) introducing into a cell: (i) a polynucleotide comprising a promoter and an engineered RNA payload disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the payload disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes may not be the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered RNA or polynucleotide encoding the engineered RNA is a non-viral vector system. In some embodiments, the non-viral vector system comprises cationic lipids, or polymers. For example, the non-viral vector system can be a liposome or polymeric nanoparticle. In some embodiments, the engineered RNA or polynucleotide encoding the engineered RNA of the disclosure or a non-viral vector comprising the engineered RNA or polynucleotide is delivered to a cell by hydrodynamic injection or ultrasound.

### Pharmaceutical Compositions

The pharmaceutical compositions described herein (e.g., compositions comprising the engineered RNAs (e.g., engineered guide RNA, ASO), the polynucleotides encoding the engineered RNAs (e.g., engineered guide RNA, ASO), or the delivery vehicles comprising the engineered RNAs or the polynucleotides encoding the engineered RNAs, all of which described here) can be formulated with a pharmaceutically acceptable carrier for administration to a subject (e.g., a human or a non-human animal) in need of the engineered RNA (e.g., engineered guide RNA, ASO), the polynucleotides, or the delivery vehicles described here, or in need of treatment of a disease or condition described here. In some embodiments, the pharmaceutical composition described here can be in a unit dose form or unit dosage form. Some embodiments of the disclosure can provide a pharmaceutical composition, comprising: (a) any of the engineered RNAs (e.g., engineered guide RNA, ASO) described here, any of the polynucleotides encoding for any of the engineered RNAs described here, or any of the delivery vehicles comprising the engineered RNAs (e.g., engineered guide RNA, ASO) or polynucleotides encoding the engineered RNAs described here; and (b) a pharmaceutically acceptable: excipient, diluent, or carrier.

As used herein, the phrase “pharmaceutically acceptable” means generally safe for ingestion or contact with biologic tissues at the levels employed. Pharmaceutically acceptable is used interchangeably with physiologically compatible.

A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. The compositions can be subjected to conventional pharmaceutical additives such as preservatives, stabilizing agents, wetting or emulsifying agents, salts for adjusting osmotic pressure, and buffers. Additional examples of carriers, stabilizers and adjuvants consistent with the compositions of the present disclosure can be found in, for example, Remington's Pharmaceutical Sciences, 21st Ed., Mack Publ. Co., Easton, Pa. (2005), incorporated herein by reference in its entirety. Such compositions as described here will, in any event, contain an effective amount of the engineered polynucleotide together with a suitable carrier so as to prepare an appropriate dosage form, unit dose form, or both for administration to a recipient subject. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form or unit dose form (e.g., a tablet, capsule, caplet, gel cap, etc.); for topical administration (e.g., as a cream, gel, lotion, or ointment); for intravenous administration (e.g., as a sterile solution free of particulate emboli and in a solvent system suitable for intravenous use); or in any other formulation described herein.

### Administration

Administration can refer to methods that can be used to enable the delivery of a composition described herein (e.g., an engineered guide RNA, ASO) to the desired site of biological action. For example, an engineered RNA (e.g., engineered guide RNA, ASO) comprising a targeting sequence sufficiently complementary to a target RNA of interest and an engineered SmOPT variant sequence, an engineered U7 hairpin variant sequence, or both the engineered SmOPT variant sequence and the engineered U7 hairpin variant sequence, or polynucleotide encoding such engineered RNA can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracorneal, intracoronary, intracorporus cavernosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method.

A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a particle, such as a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule.

In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the uses or methods of the disclosure can treat or prevent a disease or condition in the subject.

### Methods of Treatment

The engineered RNA (e.g., engineered guide RNA, ASO) described here comprising a targeting sequence with sufficient complementarity to a target RNA and operably linked to RNA elements (e.g., engineered SmOPT variant sequence, engineered U7 hairpin variant sequence, or both such as an element having a polynucleotide sequence of any one of SEQ ID NO: 49, SEQ ID NO: 50, SEQ ID NO: 51, SEQ ID NO: 60, SEQ ID NO: 61, or SEQ ID NO: 62) can be directed to a target RNA that is associated with or implicated in a disease or condition, where the target RNA associated in a disease can cause, or partially cause, or contribute to one or more symptoms of the associated disease or condition, or can be associated with or implicated in a pathway that manifests in the disease or condition. In some embodiments, the engineered guide RNAs of the disclosure comprising an engineered SmOPT variant sequence, an engineered U7 hairpin variant sequence, or both the engineered SmOPT variant sequence and the engineered U7 hairpin variant sequence, can be used to treat a disease or condition selected from: a neurodegenerative disease or disorder, a muscular disease or disorder, a metabolic disease or disorder, an ocular disease or disorder, a liver disease or disorder, a cancer, and any combination thereof. In some aspects, the disease or condition can be selected from: Rett syndrome, Huntington's disease, Parkinson's Disease, Alzheimer's disease, a muscular dystrophy, and Tay-Sachs Disease.

In some embodiments, the described engineered RNAs (e.g., engineered guide RNA, ASO) can comprise a targeting sequence with target complementarity to a target RNA of interest that can be operably linked to an engineered SmOPT variant sequence, an engineered U7 hairpin variant sequence, or both the engineered SmOPT variant sequence and the engineered U7 hairpin variant sequence, where the target RNA can be selected from the group consisting of: ABCA4, ALAS1, APP, ATP7B, CFTR, DMD, DMPK, DUX4, GAPDH, GBA, HEXA, HFE, LIPA, LRRK2, MAPT, GRN, PCSK9 start site, PINK1, PMP22, SERPINA1, SCNN1A start site, SNCA, or SOD1, a fragment of any one of these, and any combination thereof. In some embodiments, the engineered guide RNAs targeting any gene of interest in need of editing, and operably linked to an engineered SmOPT variant sequence, an engineered U7 hairpin variant sequence, or both the engineered SmOPT variant sequence and the engineered U7 hairpin variant sequence, and compositions comprising such engineered guide RNAs of the disclosure, can be useful in treating diseases or conditions associated with any of the target RNAs of interest.

Other embodiments of the disclosure can provide for a method of treating a disease or condition in a subject, the method comprising: administering to the subject an effective amount of any of the engineered RNAs (e.g., engineered guide RNA, ASO) comprising a targeting sequence with sufficient complementarity to a target RNA that can be operably linked to an engineered SmOPT variant sequence, an engineered U7 hairpin variant sequence, or both the engineered SmOPT variant sequence and the engineered U7 hairpin variant sequence described here; any polynucleotides encoding any of the engineered RNAs (e.g., engineered guide RNA, ASO) of the disclosure; any delivery vehicles described here; or any of the pharmaceutical compositions of the disclosure, to treat the disease or condition in the subject in need thereof. In some aspects, use of any of the described engineered RNAs (e.g., engineered guide RNA, ASO), any of the described polynucleotides encoding any of the engineered RNAs, any of the described delivery vehicles comprising any of the engineered RNAs or polynucleotides encoding any of the engineered RNAs, or pharmaceutical compositions comprising any of the described engineered RNAs, any of the described polynucleotides encoding any of the engineered RNAs, or any of the described delivery vehicles comprising any of the engineered RNAs or polynucleotides encoding any of the engineered RNAs described here can be as a medicament or can be used for treating a disease or condition in a subject. Such engineered RNA medicaments can be used to treat a disease or condition in a subject in need thereof. Other examples of the disclosure provide for any of the described engineered RNAs for use in the treatment of a disease or condition in a subject described here. The uses and methods disclosed here can be directed to treating a disease or condition, where treating a disease or condition can also include preventing a disease or condition, preventing or treating one or more symptoms of the disease or condition, or preventing or treating a pathway or component of a pathway that manifests in the disease or condition. In some embodiments, the uses and methods described here can be directed to treating a disease or condition selected from a group consisting of: a neurodegenerative disease or disorder, a muscular disease or disorder, a metabolic disease or disorder, an ocular disease or disorder, a liver disease or disorder, a cancer, and any combination thereof. Other diseases or conditions of the disclosure for treatment can be selected from a group consisting of: Duchenne's Muscular Dystrophy (DMD), Becker muscular dystrophy, myotonic dystrophy, Facioscapulohumeral muscular dystrophy, Rett's syndrome, Charcot-Marie-Tooth disease, Alzheimer's disease, a tauopathy, Parkinson's disease, alpha-1 antitrypsin deficiency, cystic fibrosis-like disease, Wilson disease, and Stargardt's disease. In some methods of the disclosure for treatment of a disease or condition with the disclosed engineered guide RNAs of the description, the disease or condition can be associated with a mutation in a gene, or RNA encoded by the gene, or the mutation is introduced into a gene, where the gene is selected from the group consisting of ABCA4, ALAS1, APP, ATP7B, ATP7B G1226R, CFTR, DMD, DMPK, DUX4, GAPDH, GBA, HEXA, HFE, HFE C282Y, LIPA, LIPA c.894 G>A, LRRK2, MAPT, PCSK9 start site, PINK1, PMP22, SERPINA1, SERPINA1 E342K, SCNN1A start site, SNCA, SOD1, a fragment of any of these, and any combination thereof.

In some embodiments of the disclosure, the uses or methods of treating as described here, can provide for various routes of administration, including but not limited to: inhalation, otic, buccal, conjunctival, dental, endocervical, endosinusial, endotracheal, enteral, epidural, extra-amniotic, extracorporeal, hemodialysis, infiltration, injection (e.g., parenchymal injection, intra-thecal injection, intra-ventricular injection, intra-cisternal injection, intravenous injection), interstitial, infraorbital, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracorneal, intracoronary, intracorporus cavernosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intranasal, intraocular, intraovarian, intrapericardial, intraperitoneal, intrapleural, intraprostatic, intrapulmonary, intrasinal, intraspinal, intraparenchymal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, stereotactic, or any combination thereof.

In some examples of the disclosure directed to any of the methods of treating a disease or condition in a subject, the subject can be a mammal, for example, a human. The subject of the disclosure can be a subject in need of treatment for a disease or condition, or the subject can be diagnosed with the disease or condition for treatment.

An engineered RNA (e.g., engineered guide RNA, ASO), a polynucleotide encoding the engineered RNA of the present disclosure, a delivery vehicle comprising an engineered RNA or a polynucleotide encoding the engineered RNA of the disclosure, or a pharmaceutical composition comprising any of these can be used in a method of treating a disease or condition or in uses for preparing a medicament or for treating a disease or condition in a subject in need thereof. Some embodiments described here are directed to the use of the engineered polynucleotide or the engineered RNA (e.g., engineered guide RNA, ASO) of the disclosure for treating a disease, disorder, or condition in a subject in need thereof. Other embodiments described here provide for a method of treating a disease or condition in a subject in need thereof, where the method comprises administering to the subject, an effective amount of: (a) any of the engineered RNAs RNA (e.g., engineered guide RNA, ASO) described here; any of the polynucleotides encoding any of the engineered RNAs RNA (e.g., engineered guide RNA, ASO) described here; any of the delivery vehicles comprising: any of the engineered RNAs described here or any of the polynucleotides encoding any of the engineered RNAs (e.g., engineered guide RNA, ASO) described here; or any of the pharmaceutical compositions comprising: any of the engineered RNAs described here, any of the polynucleotides comprising any of the engineered RNAs (e.g., engineered guide RNA, ASO) described here, or any of the delivery vehicles comprising any of the engineered RNAs (e.g., engineered guide RNA, ASO) described here or any of the polynucleotides encoding any of the engineered RNAs (e.g., engineered guide RNA, ASO) described here; and (b) a pharmaceutically acceptable: excipient, diluent, or carrier, where the method treats the disease or condition in the subject. A disorder can be a disease, a condition, a genotype, a phenotype, or any state associated with an adverse effect. In some embodiments, treating a disease, condition, or disorder can comprise preventing, slowing progression of, reversing, or alleviating symptoms of the disease, condition, or disorder. A method of treating a disease, disorder, or condition can comprise in some embodiments, administering or delivering: any of the engineered RNAs (e.g., engineered guide RNA, ASO) of the disclosure; any of the engineered polynucleotides encoding for any of the engineered RNAs (e.g., engineered guide RNA, ASO) of the disclosure; any of the delivery vehicles comprising any of the engineered RNAs (e.g., engineered guide RNA, ASO) or any of the polynucleotides encoding the engineered RNAs (e.g., engineered guide RNA, ASO) described here; or any of the pharmaceutical compositions comprising: any of the engineered RNAs of the disclosure; any of the engineered polynucleotides encoding for any of the engineered RNAs (e.g., engineered guide RNA, ASO) of the disclosure; any of the delivery vehicles comprising any of the engineered RNAs (e.g., engineered guide RNA, ASO) or any of the polynucleotides encoding the engineered RNAs (e.g., engineered guide RNA, ASO) described here, and a pharmaceutically acceptable: excipient, diluent, or carrier, where the method comprises administering or delivering any of the aforementioned to a subject or a cell of a subject in need thereof, to treat the disease, disorder, or condition in the subject. In some examples, the methods of treating a disease or condition

In some embodiments, a method of treating a disease, disorder, or condition can comprise, administering or delivering any of the engineered polynucleotides encoding for any of the engineered RNAs (e.g., engineered guide RNA, ASO) to a subject or a cell of a subject in need thereof expressing the engineered RNA in the subject or the cell of the subject in need thereof, to treat the disease, disorder, or condition in the subject. In some embodiments, an engineered RNA (e.g., engineered guide RNA, ASO) of the present disclosure can be used to treat a genetic disorder (e.g., FSHD, DM1, CMT1A, or ALS). In some embodiments, an engineered guide RNA of the present disclosure can be used to treat a condition associated with one or more mutations. For example, disclosed herein are methods of treating FSHD with engineered guide RNAs targeting DUX4 and further comprising an engineered SmOPT variant sequence, an engineered U7 hairpin variant sequence, or both the engineered SmOPT variant sequence and the engineered U7 hairpin variant sequence. Also disclosed herein are methods of treating DM1 with engineered guide RNAs targeting DMPK and further comprising an engineered SmOPT variant sequence, an engineered U7 hairpin variant sequence, or both the engineered SmOPT variant sequence and the engineered U7 hairpin variant sequence. Also disclosed herein are methods of treating CMT1A with engineered guide RNAs targeting PMP22 and further comprising an engineered SmOPT variant sequence, an engineered U7 hairpin variant sequence, or both the engineered SmOPT variant sequence and the engineered U7 hairpin variant sequence. Also disclosed herein are methods of treating ALS with engineered guide RNAs targeting SOD1 and further comprising an engineered SmOPT variant sequence, an engineered U7 hairpin variant sequence, or both the engineered SmOPT variant sequence and the engineered U7 hairpin variant sequence.

In some embodiments, the engineered RNA of the present disclosure (engineered guide RNA, ASO) having an engineered SmOPT variant sequence, an engineered U7 hairpin variant sequence, or both facilitates an RNA edit (e.g., by ADAR) that results in exon skipping. In some embodiments, the engineered RNA of the present disclosure (engineered guide RNA, ASO) having an engineered SmOPT variant sequence, an engineered U7 hairpin variant sequence, or both can bind to and mask a sequence of a target RNA, thereby promoting exon skipping. In some embodiments, the engineered RNA of the present disclosure (engineered guide RNA, ASO) having an engineered SmOPT variant sequence, an engineered U7 hairpin variant sequence, or both, can facilitate an RNA edit (e.g., by ADAR) and can bind to and mask a sequence of a target RNA; and thus exhibits an additive increase in exon skipping. The engineered RNA of the present disclosure can improve exon skipping as compared to polynucleotide constructs not containing an engineered SmOPT variant sequence or an engineered U7 hairpin variant sequence. The exon may contain a mutation that alters its function. The engineered RNA of the present disclosure can have improved exon skipping as compared to polynucleotide constructs not containing an engineered SmOPT variant sequence or an engineered U7 hairpin variant sequence when measured in vitro. Efficiency of exon skipping can be measured by quantitative PCR, droplet digital PCR, or RNA sequencing.

The exon skipping efficiency of the engineered RNA can be measured by an in vitro assay, such as a quantitative PCR assay or droplet digital PCR assay to detect the proportion of exon-skipped transcripts relative to the proportion of unskipped transcripts. In this assay, at least two fluorophore-conjugated probes are used, one which specifically anneals to an exon-skipped transcript and another which specifically anneals to an unskipped transcript. ddPCR amplification was performed.

The engineered RNA of the present disclosure can increase the efficiency of exon skipping by at least 1%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or up to 100%, as measured by an ddPCR. The engineered polynucleotides of the present disclosure can increase the efficiency of exon skipping by about 1% to about 50%. The engineered RNA of the present disclosure can increase the efficiency of exon skipping by at least about 1%. The engineered RNA of the present disclosure can increase the efficiency of exon skipping by at most about 50%. The engineered polynucleotides of the present disclosure can increase the efficiency of exon skipping by about 1% to about 5%, about 1% to about 10%, about 1% to about 20%, about 1% to about 30%, about 1% to about 40%, about 1% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50%.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

As used herein, the term “polynucleotide” can refer to a single or double-stranded polymer of deoxyribonucleotide (DNA) or ribonucleotide (RNA) bases read from the 5′ to the 3′ end. The term “RNA” is inclusive of dsRNA (double stranded RNA), guide-target RNA scaffolds formed upon hybridization of an engineered RNA and target RNA (where the guide-target RNA scaffold can have at least one, two, or more structural features, such as but not limited to, a bulge, a mismatch an internal loop, a hairpin, or a wobble base pair), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

As used herein, the term “facilitates RNA editing” by an engineered guide RNA refers to the ability of the engineered guide RNA, when associated with an RNA editing entity, and a target RNA to provide a targeted edit of the target RNA by the RNA edited entity. In some instances, the engineered RNA can directly recruit, position, orient, or any combinations thereof, the RNA editing entity to the proper location for editing of the target RNA. In other instances, the engineered guide RNA upon hybridization to the target RNA forms a guide-target RNA scaffold with one or more features as described herein, where the guide-target RNA scaffold with the features recruits, positions, orients, or any combinations thereof, the RNA editing entity to the proper location for editing of the target RNA.

Detailed embodiments are disclosed herein; however, it is to be understood that the disclosed embodiments are merely illustrative. In addition, each of the examples given in connection with the various embodiments is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 0.3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.10% to 2.5%, etc. It will be understood that the sum of all weight % of individual components will not exceed 100%.

A double stranded RNA (dsRNA) substrate (e.g., a guide-target RNA scaffold) is formed upon hybridization of an engineered guide RNA of the present disclosure to a target RNA. As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can have from 0 to 4 contiguous nucleotides on the guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

As described here, a double stranded RNA (dsRNA) substrate (e.g., a guide-target RNA scaffold) is formed upon hybridization of an engineered guide RNA of the present disclosure to a target RNA. A bulge can be a symmetrical bulge or an asymmetrical bulge. A “symmetrical bulge” is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

As disclosed here, a double stranded RNA (dsRNA) substrate (e.g., a guide-target RNA scaffold) is formed upon hybridization of an engineered guide RNA of the present disclosure to a target RNA. A bulge can be a symmetrical bulge or an asymmetrical bulge. An “asymmetrical bulge” is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

A “barbell” as described herein refers to a pair of internal loop latent structures that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint, which in turn comprises at least one structural feature that facilitates editing of a specific target RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (e.g., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

By “consist essentially” it is meant that the ingredients include only the listed components along with the normal impurities present in commercial materials and with any other additives present at levels which do not affect operation of embodiments provided in the present disclosure, for instance at levels less than 5% by weight or less than 1% or even 0.5% by weight.

The terms “determining,” “measuring,” “evaluating,” “assessing,” “assaying,” and “analyzing” can be used interchangeably herein to refer to forms of measurement. The terms include determining if an element can be present or not (for example, detection). These terms can include quantitative, qualitative or quantitative and qualitative determinations. Assessing can be relative or absolute. “Detecting the presence of” can include determining the amount of something present in addition to determining whether it can be present or absent depending on the context.

The term “effective amount” or “therapeutically effective amount” of an agent or a composition (e.g., compositions having an engineered polynucleotide, etc.), as used herein, is that amount sufficient to effect beneficial or desired results, such as clinical results, and, as such, an “effective amount” depends upon the context in which it is being applied. In some embodiments, the compositions are administered in an effective amount for the treatment or prophylaxis of a disease disorder or condition. In another embodiment, administering an effective amount of a composition is, for example, an amount sufficient to achieve alleviation or amelioration or prevention or prophylaxis of one or more symptoms or conditions; diminishment of the extent of the disease, disorder, or condition; stabilized (maintaining or not worsening) the state of the disease, disorder, or condition; preventing the spread of the disease, disorder, or condition; delaying or slowing the progress of the disease, disorder, or condition; amelioration or palliation of the disease, disorder, or condition; and remission (whether partial or total), whether detectable or undetectable, as compared to the response obtained without administration of the agent. “Palliating” a disease, disorder, or condition means that the extent of the disease, disorder, or condition, undesirable clinical manifestations of the disease, disorder, or condition, or both are lessened, time course of the progression is slowed or lengthened, or both, as compared to the extent or time course in the absence of treatment.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

As disclosed here, a double stranded RNA (dsRNA) substrate is formed upon hybridization of an engineered guide RNA of the present disclosure to a target RNA. The resulting dsRNA substrate is also referred to herein as a “guide-target RNA scaffold.” Described herein are “structural features” that can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more features selected from the group consisting of a bulge, mismatch, internal loop, hairpin, wobble base pair, and any combination thereof.

As disclosed herein, a “hairpin” is an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

As disclosed herein, a hairpin can refer to a recruitment hairpin, a non-recruitment hairpin, or any combination thereof. A “recruitment hairpin,” as disclosed herein, can recruit at least in part an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that can be present in constructs comprising an engineered guide RNA, not a structural feature formed by latent structure provided in an engineered latent guide RNA. A recruitment hairpin, as described herein, can be a naturally occurring ADAR substrate or truncations thereof.

A “non-recruitment hairpin,” as disclosed herein, does not have a primary function of recruiting an RNA editing entity. A non-recruitment hairpin, in some instances, does not recruit an RNA editing entity. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)) and incorporated herein by reference for its teachings in its entirety. Software for performing BLAST analyses is publicly available through the National Center for Biotechnology Information.

In some embodiments, a double stranded RNA (dsRNA) substrate (e.g., a guide-target RNA scaffold) is formed upon hybridization of an engineered guide RNA of the present disclosure to a target RNA. As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered polynucleotide side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

As described here, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. A “symmetrical internal loop” is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

As disclosed here, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered polynucleotide side of the guide-target RNA scaffold. An asymmetrical internal loop of the present disclosure can be formed by 5 nucleotides on the target RNA side of the guide-target RNA scaffold and 100 nucleotides on the engineered polynucleotide side of the guide-target RNA scaffold. An asymmetrical internal loop of the present disclosure can be formed by 5 nucleotides on the target RNA side of the guide-target RNA scaffold and 150 nucleotides on the engineered polynucleotide side of the guide-target RNA scaffold. An asymmetrical internal loop of the present disclosure can be formed by 5 nucleotides on the target RNA side of the guide-target RNA scaffold and 200 nucleotides on the engineered polynucleotide side of the guide-target RNA scaffold. An asymmetrical internal loop of the present disclosure can be formed by 5 nucleotides on the target RNA side of the guide-target RNA scaffold and 300 nucleotides on the engineered polynucleotide side of the guide-target RNA scaffold. An asymmetrical internal loop of the present disclosure can be formed by 5 nucleotides on the target RNA side of the guide-target RNA scaffold and 400 nucleotides on the engineered polynucleotide side of the guide-target RNA scaffold. An asymmetrical internal loop of the present disclosure can be formed by 5 nucleotides on the target RNA side of the guide-target RNA scaffold and 500 nucleotides on the engineered polynucleotide side of the guide-target RNA scaffold. An asymmetrical internal loop of the present disclosure can be formed by 5 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 5 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 5 nucleotides on the engineered polynucleotide side of the guide-target RNA scaffold. An asymmetrical internal loop of the present disclosure can be formed by 300 nucleotides on the target RNA side of the guide-target RNA scaffold and 5 nucleotides on the engineered polynucleotide side of the guide-target RNA scaffold. An asymmetrical internal loop of the present disclosure can be formed by 200 nucleotides on the target RNA side of the guide-target RNA scaffold and 5 nucleotides on the engineered polynucleotide side of the guide-target RNA scaffold. An asymmetrical internal loop of the present disclosure can be formed by 150 nucleotides on the target RNA side of the guide-target RNA scaffold and 5 nucleotides on the engineered polynucleotide side of the guide-target RNA scaffold. An asymmetrical internal loop of the present disclosure can be formed by 100 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 100 nucleotides on the engineered polynucleotide side of the guide-target RNA scaffold. An asymmetrical internal loop of the present disclosure can be formed by 50 nucleotides on the target RNA side of the guide-target RNA scaffold and 150 nucleotides on the engineered polynucleotide side of the guide-target RNA scaffold. An asymmetrical internal loop of the present disclosure can be formed by 50 nucleotides on the target RNA side of the guide-target RNA scaffold and 200 nucleotides on the engineered polynucleotide side of the guide-target RNA scaffold. An asymmetrical internal loop of the present disclosure can be formed by 50 nucleotides on the target RNA side of the guide-target RNA scaffold and 300 nucleotides on the engineered polynucleotide side of the guide-target RNA scaffold. An asymmetrical internal loop of the present disclosure can be formed by 50 nucleotides on the target RNA side of the guide-target RNA scaffold and 400 nucleotides on the engineered polynucleotide side of the guide-target RNA scaffold. An asymmetrical internal loop of the present disclosure can be formed by 50 nucleotides on the target RNA side of the guide-target RNA scaffold and 500 nucleotides on the engineered polynucleotide side of the guide-target RNA scaffold. An asymmetrical internal loop of the present disclosure can be formed by 50 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 50 nucleotides on the engineered polynucleotide side of the guide-target RNA scaffold. An asymmetrical internal loop of the present disclosure can be formed by 500 nucleotides on the target RNA side of the guide-target RNA scaffold and 50 nucleotides on the engineered polynucleotide side of the guide-target RNA scaffold. An asymmetrical internal loop of the present disclosure can be formed by 400 nucleotides on the target RNA side of the guide-target RNA scaffold and 50 nucleotides on the engineered polynucleotide side of the guide-target RNA scaffold. An asymmetrical internal loop of the present disclosure can be formed by 300 nucleotides on the target RNA side of the guide-target RNA scaffold and 50 nucleotides on the engineered polynucleotide side of the guide-target RNA scaffold. An asymmetrical internal loop of the present disclosure can be formed by 200 nucleotides on the target RNA side of the guide-target RNA scaffold and 50 nucleotides on the engineered polynucleotide side of the guide-target RNA scaffold. An asymmetrical internal loop of the present disclosure can be formed by 150 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 150 nucleotides on the engineered polynucleotide side of the guide-target RNA scaffold. An asymmetrical internal loop of the present disclosure can be formed by 100 nucleotides on the target RNA side of the guide-target RNA scaffold and 200 nucleotides on the engineered polynucleotide side of the guide-target RNA scaffold. An asymmetrical internal loop of the present disclosure can be formed by 100 nucleotides on the target RNA side of the guide-target RNA scaffold and 300 nucleotides on the engineered polynucleotide side of the guide-target RNA scaffold. An asymmetrical internal loop of the present disclosure can be formed by 100 nucleotides on the target RNA side of the guide-target RNA scaffold and 400 nucleotides on the engineered polynucleotide side of the guide-target RNA scaffold. An asymmetrical internal loop of the present disclosure can be formed by 100 nucleotides on the target RNA side of the guide-target RNA scaffold and 500 nucleotides on the engineered polynucleotide side of the guide-target RNA scaffold. An asymmetrical internal loop of the present disclosure can be formed by 100 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 100 nucleotides on the engineered polynucleotide side of the guide-target RNA scaffold. An asymmetrical internal loop of the present disclosure can be formed by 500 nucleotides on the target RNA side of the guide-target RNA scaffold and 100 nucleotides on the engineered polynucleotide side of the guide-target RNA scaffold. An asymmetrical internal loop of the present disclosure can be formed by 400 nucleotides on the target RNA side of the guide-target RNA scaffold and 100 nucleotides on the engineered polynucleotide side of the guide-target RNA scaffold. An asymmetrical internal loop of the present disclosure can be formed by 300 nucleotides on the target RNA side of the guide-target RNA scaffold and 100 nucleotides on the engineered polynucleotide side of the guide-target RNA scaffold. An asymmetrical internal loop of the present disclosure can be formed by 200 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 200 nucleotides on the engineered polynucleotide side of the guide-target RNA scaffold. An asymmetrical internal loop of the present disclosure can be formed by 150 nucleotides on the target RNA side of the guide-target RNA scaffold and 300 nucleotides on the engineered polynucleotide side of the guide-target RNA scaffold. An asymmetrical internal loop of the present disclosure can be formed by 150 nucleotides on the target RNA side of the guide-target RNA scaffold and 400 nucleotides on the engineered polynucleotide side of the guide-target RNA scaffold. An asymmetrical internal loop of the present disclosure can be formed by 150 nucleotides on the target RNA side of the guide-target RNA scaffold and 500 nucleotides on the engineered polynucleotide side of the guide-target RNA scaffold. An asymmetrical internal loop of the present disclosure can be formed by 150 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 150 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 150 nucleotides on the engineered polynucleotide side of the guide-target RNA scaffold. An asymmetrical internal loop of the present disclosure can be formed by 300 nucleotides on the target RNA side of the guide-target RNA scaffold and 150 nucleotides on the engineered polynucleotide side of the guide-target RNA scaffold. An asymmetrical internal loop of the present disclosure can be formed by 200 nucleotides on the target RNA side of the guide-target RNA scaffold and 300 nucleotides on the engineered polynucleotide side of the guide-target RNA scaffold. An asymmetrical internal loop of the present disclosure can be formed by 200 nucleotides on the target RNA side of the guide-target RNA scaffold and 400 nucleotides on the engineered polynucleotide side of the guide-target RNA scaffold. An asymmetrical internal loop of the present disclosure can be formed by 200 nucleotides on the target RNA side of the guide-target RNA scaffold and 500 nucleotides on the engineered polynucleotide side of the guide-target RNA scaffold. An asymmetrical internal loop of the present disclosure can be formed by 200 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 200 nucleotides on the engineered polynucleotide side of the guide-target RNA scaffold. An asymmetrical internal loop of the present disclosure can be formed by 500 nucleotides on the target RNA side of the guide-target RNA scaffold and 200 nucleotides on the engineered polynucleotide side of the guide-target RNA scaffold. An asymmetrical internal loop of the present disclosure can be formed by 400 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 400 nucleotides on the engineered polynucleotide side of the guide-target RNA scaffold. An asymmetrical internal loop of the present disclosure can be formed by 300 nucleotides on the target RNA side of the guide-target RNA scaffold and 500 nucleotides on the engineered polynucleotide side of the guide-target RNA scaffold. An asymmetrical internal loop of the present disclosure can be formed by 300 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 300 nucleotides on the engineered polynucleotide side of the guide-target RNA scaffold. An asymmetrical internal loop of the present disclosure can be formed by 500 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 500 nucleotides on the engineered polynucleotide side of the guide-target RNA scaffold. An asymmetrical internal loop of the present disclosure can be formed by 400 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 500 nucleotides on the engineered polynucleotide side of the guide-target RNA scaffold. Thus, an asymmetrical internal loop can be a structural feature formed from latent structure provided by an engineered latent guide RNA.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As disclosed herein, a “macro-footprint” sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As described herein, a “micro-footprint” sequence refers to a sequence with latent structures that, when manifested, facilitate editing of the adenosine of a target RNA via an adenosine deaminase enzyme.

A double stranded RNA (dsRNA) substrate (e.g., a guide-target RNA scaffold) is formed upon hybridization of an engineered guide RNA of the present disclosure to a target RNA. As disclosed herein, the term “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA. In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

The term “mutation” as used herein, can refer to an alteration to a nucleic acid sequence or a polypeptide sequence that can be relative to a reference sequence. A mutation can occur in a DNA molecule, a RNA molecule (e.g., tRNA, mRNA), or in a polypeptide or protein, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. Non-limiting examples of mutations in a nucleic acid sequence that, without the mutation, encodes for a polypeptide sequence, include: “missense” mutations that can result in the substitution of one codon for another, “nonsense” mutations that can change a codon from one encoding a particular amino acid to a stop codon (which can result in truncated translation of proteins), or “silent” mutations that can be those which have no effect on the resulting protein. The mutation can be a “point mutation,” which can refer to a mutation affecting only one nucleotide in a DNA or RNA sequence. The mutation can be a “splice site mutations,” which can be present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can comprise a sequence variant, a sequence variation, a sequence alteration, or an allelic variant. The mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (e.g., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample is benign. As an alternative, absence of a mutation may not indicate that a tissue or sample is benign. Methods as described herein can comprise identifying a presence of a mutation in a sample.

A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample can be benign. As an alternative, absence of a mutation may not indicate that a tissue or sample can be benign. Methods as described herein can comprise identifying a presence of a mutation in a sample.

The terms “polynucleotide” and “oligonucleotide” can be used interchangeably and can refer to a polymeric form of nucleotides of any length, either deoxyribonucleotides or ribonucleotides or analogs thereof. Polynucleotides can have any three-dimensional structure and can perform any function, known or unknown. The following may be non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term can also refer to both double- and single-stranded molecules. Unless otherwise specified or required, any embodiment of this disclosure that can be a polynucleotide encompasses both the double-stranded form and each of two complementary single-stranded forms known or predicted to make up the double-stranded form.

A polynucleotide can be composed of a specific sequence of nucleotides. A nucleotide comprises a nucleoside and a phosphate group. A nucleotide comprises a sugar (e.g., ribose or 2′ deoxyribose) and a nucleobase, such as a nitrogenous base. Non-limiting examples of nucleobases include adenine (A), cytosine (C), guanine (G), thymine (T), uracil (U), and inosine (I). In some embodiments, I can be formed when hypoxanthine can be attached to ribofuranose via a P-N9-glycosidic bond, resulting in the chemical structure:

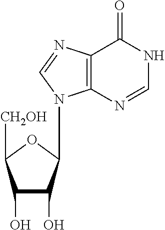

Some polynucleotide embodiments refer to a DNA sequence. In some embodiments, the DNA sequence can be interchangeable with a similar RNA sequence. Some embodiments refer to an RNA sequence. In some embodiments, the RNA sequence can be interchangeable with a similar DNA sequence. In some embodiments, Us and Ts can be interchanged in a sequence provided herein.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “stop codon” can refer to a three nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

The term “structured motif,” as disclosed herein, comprises two or more features in a guide-target RNA scaffold.

As used herein, the term “subject” refers to any organism to which a composition (e.g., composition comprising an engineered RNA of the disclosure), agent, or both in accordance with the disclosure can be administered, e.g., for experimental, diagnostic, prophylactic, therapeutic purposes, or combinations thereof. Typical subjects include any animal (e.g., mammals such as mice, rats, rabbits, non-human primates, and humans, etc.). A subject in need thereof is typically a subject for whom it is desirable to treat a disease, disorder, or condition as described herein or treat with a composition described herein. For example, a subject in need thereof can seek or be in need of treatment, require treatment, be receiving treatment, can be receiving treatment in the future, or a human or animal that is under care by a trained professional for a particular disease, disorder, or condition.

The term “in vivo” refers to an event that takes place in a subject's body.

The term “ex vivo” refers to an event that takes place outside of a subject's body. An ex vivo assay may be not performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly base pair. For example, a wobble base pair of the present disclosure can refer to a G paired with a U. Thus, a wobble base pair can be a structural feature formed from latent structure provided by an engineered latent guide RNA.

As used herein, the terms “treatment” or “treating” can be used in reference to a pharmaceutical or other intervention regimen for obtaining beneficial or desired results in the recipient. Beneficial or desired results include but may not be limited to a therapeutic benefit, a prophylactic benefit, or both. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### EXAMPLES

The following illustrative examples are representative of embodiments of the stimulation, systems, and methods described herein and are not meant to be limiting in any way.

### Example 1

### Mutagenesis Screening for SmOPT and U7 Hairpins

This example describes a mutagenesis screen of exemplary engineered polynucleotide guide RNAs containing variations in the SmOPT sequence, a natural U7 Sm binding sequence, a natural U1 Sm binding sequence, a mouse U7 hairpin, or a human U7 hairpin. This set of 254 SmOPT U7 sequence variants will be appended to three antisense guide RNAs: 100 nucleotides targeting SNCA 3′UTR (SNCA 3′UTR 100@50; SEQ ID NO: 17); RAB7A 3′UTR hnRNPA1 (RAB7A 3′UTR hnRNPA1 100@50; SEQ ID NO: 18); or 115 nucleotides targeting GAPDH (GAPDH 115@80 4 loops (−5,+30); SEQ ID NO: 19).

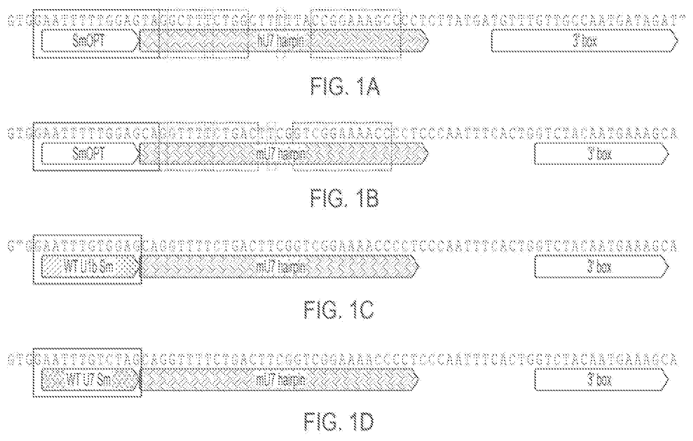

FIG. 1A-FIG. 1D show exemplary engineered polynucleotides containing an Sm binding domain sequence and a U7 hairpin sequence. The solid black boxes of FIG. 1A and FIG. 1B identify the areas where 14 single base substitutions are performed. The black dotted boxes of FIG. 1A and FIG. 1B represent the areas where 10 or 14 hairpin paired substitutions are performed, respectively. The black dashed boxes of FIG. 1A and FIG. 1B represent the areas where an extra duplicate base is inserted. The solid black boxes of FIG. 1C and FIG. 1D indicate the areas where 12 single base substitutions are performed. The number of potential substitutions (e.g., 76) with 3 alternate bases for each substitution results in 228 total mutants. Adding in the duplicate base variants and control sequences, yields 253 SmOPT U7 variants. Using, for example, the three guide RNAs appended to the total set of mutants generates a 759-plex library screen.

RNA editing resulting from each guide RNA in the guide RNA library with the SmOPT sequence and U7 hairpin variations is measured. 293T cells are transfected with plasmid comprising the pool of engineered guide RNA constructs; exactly one construct from the library will be stably integrated into the genome of each transfected cell and the editing efficiency of each guide RNA is measured 12 days later.

### Example 2

### Mutagenesis Screening of SmOPT, U1Sm, U7Sm and U7 Hairpins

Guide RNAs with individually mutated bases in the SmOPT sequence, the U1Sm sequence, the U7Sm sequence, the mouse U7 (mU7) hairpin sequence, or the human U7 (hU7) hairpin sequence were tested to determine editing efficiency as compared to wild-type constructs. The unmodified SmOPT sequence (AATTTTTGGAG SEQ ID NO: 22) (RNA Sequence AAUUUUUGGAG SEQ ID NO: 2), unmodified U1Sm sequence (AATTTGTGGAG SEQ ID NO: 23) (RNA Sequence AAUUUGUGGAG SEQ ID NO: 20), and unmodified U7Sm sequence (AATTTGTCTAG SEQ ID NO: 24) (RNA Sequence AAUUUGUCUAG SEQ ID NO: 21) were mutated individually at each base in the DNA encoding the guide RNA. The unmodified mU7 hairpin sequence (SEQ ID NO: 3) (partial mU7 DNA Sequence, CAGGTTTTCTGAC SEQ ID NO: 25) (partial RNA Sequence, CAGGUUUUCUGAC SEQ ID NO: 26) and the unmodified hU7 hairpin sequence (SEQ ID NO: 4) (partial hU7 DNA Sequence, TAGGCTTTCTGG SEQ ID NO: 27) (partial RNA Sequence UAGGCUUUCUGG SEQ ID NO: 28) were mutated individually at each of the first two bases or as complementary pairs along the hairpin stem-loop region in the DNA encoding the guide RNA. This set of variations was appended to the antisense regions for three target RNAs: RAB7A, SNCA, and GAPDH to determine the mutation's effect on the editing efficacy.

Guides were tested by a single-cell single-copy pooled-library transfection screen. Briefly, a plasmid library pool of guide RNAs carrying mutations in the Sm-binding or U7 hairpin domains, as described above and illustrated in FIG. 1, was assembled. Illumina sequencing of these plasmid library pools matched each mutated guide RNA with an identifying moiety. 293T cells were transfected with the plasmid library pools. Successful single-copy integrations were identified by fluorescence expression and selected for. Editing efficacy for each guide RNA was measured 13 days after transfection.

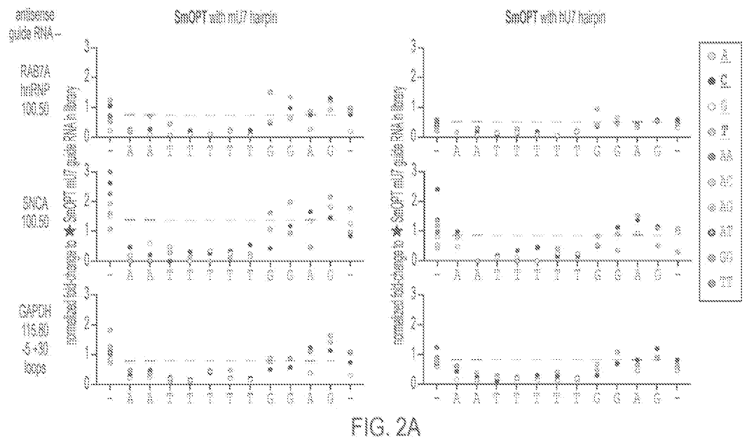

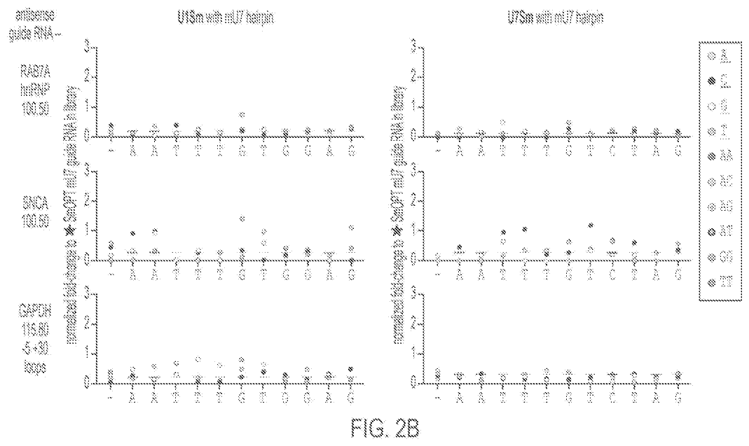

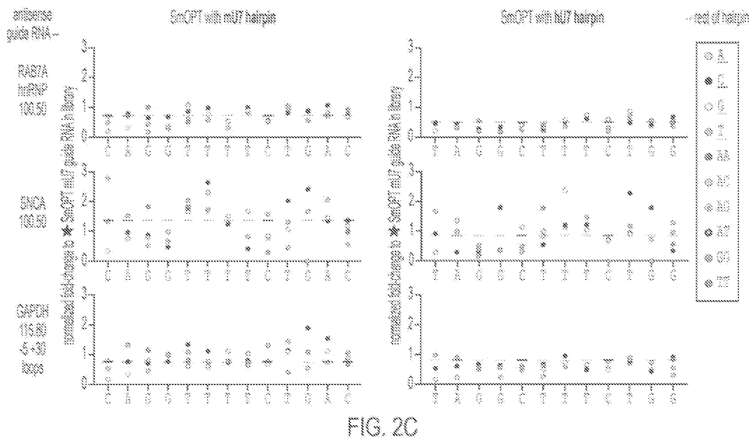

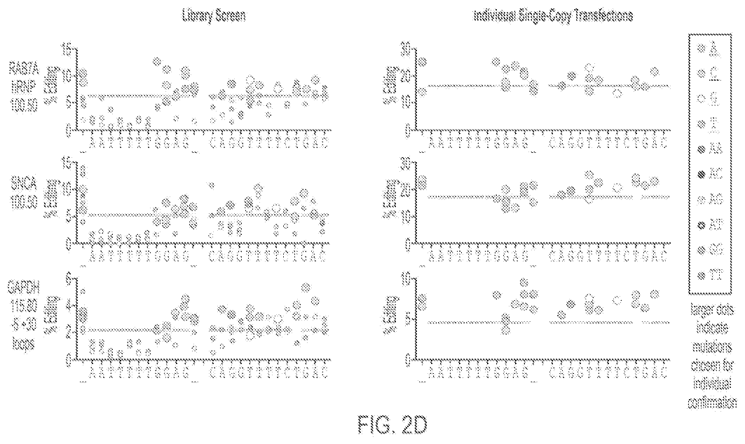

For each mutation, RNA editing of the target RNA from each mutated or unmodified guide was normalized to that of the unmodified SmOPT-mU7 hairpin guide and plotted as a fold change. FIG. 2A-FIG. 2D show editing of the targeted transcripts by their respective guide RNAs: RAB7A hnRNP, SNCA, and GAPDH. The X-axis on the graphs lists the unmodified sequence of SmOPT, U1Sm, U7Sm, mU7 and hU7, with additional spaces for an inserted base before or after the Sm binding domain. For FIG. 2A-FIG. 2C, the Y-axis on the graphs shows the normalized fold-change in editing relative to the parent (unmodified) guide RNA. The mutations for each nucleotide are shown as individual circle symbols with representative patterns. The editing level of the unmodified guide RNA is shown as a dashed line symbol across each position. The mutated RNA guide sequences in FIGS. 2A-2D are presented as the DNA sequences encoding the RNA guide sequences. Guide RNA sequences can be determined by substituting a U for a T in the presented nucleotide sequences. FIG. 2A shows guide RNA mutations of the SmOPT sequence (AATTTTTGGAG SEQ ID NO: 22) with the mouse U7 (mU7) hairpin or the human U7 (hU7) hairpin sequence. The mutation's effect on the fold-change editing efficiency was compared to the unmodified SmOPT guide RNA. Mutations in any T of the TTTTT region (corresponding to nucleotides in 3-7) resulted in decreased editing. An A mutation at residue 8 of the SmOPT sequence corresponding to nucleotide G, resulted in increased editing efficiency for the RAB7A hnRNP target in both the mU7 and hU7 constructs. In another example, a T mutation at residues 8, 9, and 11 of the SmOPT sequence corresponding to nucleotides G, G and G respectively, resulted in increased editing efficiency for the SNCA target in the mU7 construct. Several insertions (e.g., an AT or C insertion for both SNCA guides, or an A insertion for the GAPDH guide with the SmOPT/mU7 construct) in the +1 sequence of the SmOPT guide resulted in increased editing. These results show the SmOPT sequence can be modified to increase editing for a specific target.

FIG. 2B shows guide RNA mutations of the U1Sm sequence (AATTTGTGGAG SEQ ID NO: 23) with the mU7 hairpin or the U7Sm sequence (AATTTGTCTAG SEQ ID NO: 24) with the mU7 hairpin. The mutations effect on the fold-change editing efficiency was compared to the unmodified guide RNA.

FIG. 2C shows guide RNA mutations of the mU7 hairpin sequence (SEQ ID NO: 3) with SmOPT or the hU7 hairpin sequence (SEQ ID NO: 4) with SmOPT. For brevity, only the partial mU7 hairpin sequence (CAGGTTTTCTGAC SEQ ID NO: 25) or the partial hU7 hairpin sequence (TAGGCTTTCTGG SEQ ID NO: 27) is listed on the X-axis. The mutation's effect of the fold-change editing efficiency was compared to the unmodified guide RNA. Several mutations in the mU7 hairpin sequence resulted in increased editing. For example, a T mutation at the 1^{st}, or the 12^{th} residue of the mU7 sequence corresponding to nucleotides C and A respectively, resulted in increased editing for the SNCA target RNA. Additionally, a C mutation in the 6^{th}, 10^{th} or the 11^{th} residue of the mU7 sequence corresponding to nucleotides T, T and G resulted in increased editing of the SNCA target RNA. For GAPDH, a C mutation in the 11^{th} or the 12^{th} residue of the mU7 sequence corresponding to nucleotides G and A resulted in increased editing of the GAPDH target RNA.

Similarly, several mutations in the hU7 hairpin sequence resulted in increased editing. For example, a C mutation in the 4^{th} 10^{th} or the 11^{th} residue of the hU7 sequence corresponding to nucleotides G, T, or G respectively, resulted in increased editing for the SNCA target RNA. Additionally, a G mutation in the 7^{th} residue corresponding to the nucleotide T resulted in increased editing of the SNCA target RNA. These results show the mU7 and hU7 sequence can be modified to increase editing for a specific target.

FIG. 2D shows a summary of mutations in the SmOPT (AATTTTTGGAG SEQ ID NO: 22) and partial mU7 (CAGGTTTTCTGAC SEQ ID NO: 25) sequences associated with increased editing in the three target RNAs (RAB7A hnRNP, SNCA, and GAPDH). The left graphs show percent editing data from the library screen; the right graphs show percent editing data from individual single-copy transfections. To confirm mutations identified by the library screen (denoted as larger circles), these were cloned as individual plasmids and each transfected into 293T cells. Successful single-copy integrations were identified by fluorescence expression and selected for testing. Editing efficacy for each guide RNA was measured 13 days after transfection.

Representative mutations that displayed increased editing efficiency were chosen for testing by the individual transfections. For the SmOPT sequence, insertion mutations before the SmOPT of T or C resulted in increased editing efficiency. Additionally, an A mutation at residue 8 of the SmOPT sequence corresponding to nucleotide G, generally resulted in increased editing efficiency; a T, C or A mutation at residue 9 of the SmOPT sequence corresponding to nucleotide G, generally resulted in increased editing efficiency; a C mutation at residue 10 of the SmOPT sequence corresponding to nucleotide A, generally resulted in increased editing efficiency; a T, C or A mutation at residue 11 of the SmOPT sequence corresponding to nucleotide G, resulted in increased editing efficiency; and an insertion mutation of A or T after the SmOPT generally resulted in increased editing efficiency. For the mU7 sequence, a T mutation at residue 2 of the mU7 sequence corresponding to nucleotide A, generally resulted in increased editing efficiency; a GG insertion mutation at residue 3 of the mU7 sequence corresponding to nucleotide G, resulted in increased editing efficiency; a G, C, or A mutation at residue 5 of the mU7 sequence corresponding to nucleotide T, generally resulted in increased editing efficiency; a C mutation at residue 6 of the mU7 sequence corresponding to nucleotide T, resulted in increased editing efficiency; a G mutation at residue 8 of the mU7 sequence corresponding to nucleotide T, resulted in increased editing efficiency; a C, or A mutation at residue 10 of the mU7 sequence corresponding to nucleotide T, generally resulted in increased editing efficiency; a C mutation at residue 11 of the mU7 sequence corresponding to nucleotide G, resulted in increased editing efficiency; and a C mutation at residue 12 of the mU7 sequence corresponding to nucleotide A, resulted in increased editing efficiency. These results show several sequence mutations of SmOPT and mU7 can be beneficial across different target sequences.

Representative mutations were confirmed by individual single-copy transfections to increase editing on at least one gene target, ten (DNA sequence SEQ ID NO: 29-SEQ ID NO: 38 or RNA sequence SEQ ID NO: 39-SEQ ID NO: 48) increased editing on all three gene targets.

### Example 3

### Testing SmOPT and U7 Hairpin Variants in Combination

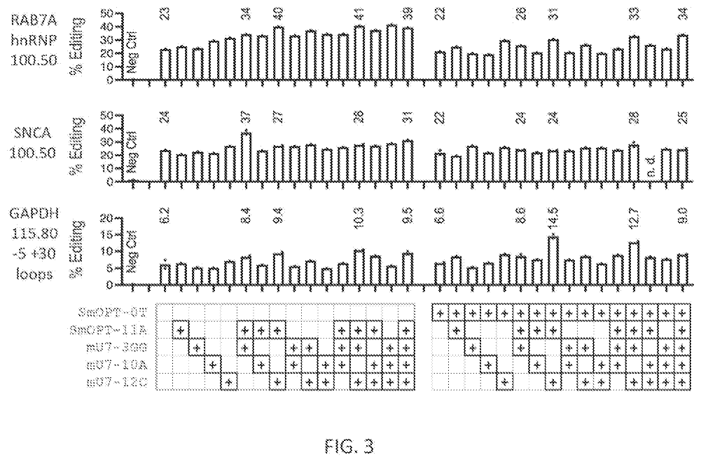

Representative mutations for increased editing efficiency were combined (DNA sequence SEQ ID NO: 49-SEQ ID NO: 59 or RNA sequence SEQ ID NO: 60-SEQ ID NO: 70) and tested by individual transfections. FIG. 3 shows RNA editing of the targeted transcripts by their respective guide RNAs: RAB7A hnRNP, SNCA, and GAPDH. 293T cells were transfected with the respective plasmid constructs. Successful single-copy integrations were identified by fluorescence expression and selected for. Editing efficacy for each guide RNA was measured 13 days after transfection. The X-axis on the graphs indicates the presence of each individual variant within the SmOPT or mU7 and hU7 hairpin sequence. Numbers listed above each bar denote the percent of RNA editing. The negative control indicates the percent of target transcript editing from a guide RNA possessing an antisense sequence from a different gene target.

Combinations of the representative individual mutations were confirmed by individual single-copy transfections to further increase editing on at least one gene target, three (DNA sequence SEQ ID NO: 49-SEQ ID NO: 51 or RNA sequence SEQ ID NO: 60-SEQ ID NO: 62) showed the highest overall gain across all three gene targets.

### Example 4

### Testing SmOPT and U7 Hairpin Combination Variants on Additional Targets

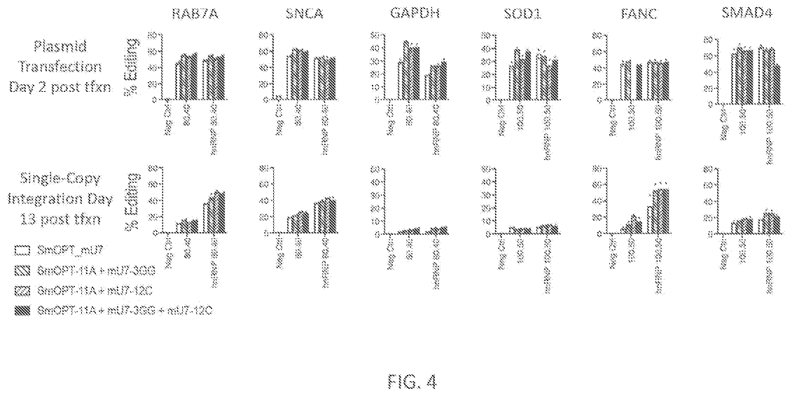

Representative variant combinations for increased editing efficiency (DNA sequence SEQ ID NO: 49-SEQ ID NO: 51 or RNA sequence SEQ ID NO: 60-SEQ ID NO: 62) were then tested by individual transfections against further gene targets. FIG. 4 shows RNA editing of the targeted transcripts by the indicated guide RNA. 293T cells were transfected with the respective plasmid constructs. Successful single-copy integrations were identified by fluorescence expression and selected for. Editing efficacy for each guide RNA was measured 2 or 13 days after transfection.

For broader confirmation of the increased editing efficiency, the SmOPT U7 hairpin variants were appended to three antisense guide RNAs in an 80@40 format, with or without a 5′ hnRNPA1 domain (SEQ ID NO: 71): RAB7A 3′UTR 80@40 (SEQ ID NO: 72); SNCA 3′UTR 80@40 (SEQ ID NO: 73); GAPDH 80@40 (SEQ ID NO: 74). In addition, the SmOPT U7 hairpin variants were appended to three antisense guide RNAs in a 100@50 format, with or without a 5′ hnRNPA1 domain (SEQ ID NO: 71): SOD1 100@50 (SEQ ID NO: 75); FANC 100@50 (SEQ ID NO: 76); SMAD4 100@50 (SEQ ID NO: 77). The negative control indicates the percent of target transcript editing from a guide RNA possessing an antisense sequence from a different gene target. For each antisense sequence, the new SmOPT U7 hairpin variant showed the same or increased RNA editing compared to the original SmOPT mU7 hairpin sequence.

### Example 5

### Testing SmOPT and U7 Hairpin Combination Variants for Exon Skipping

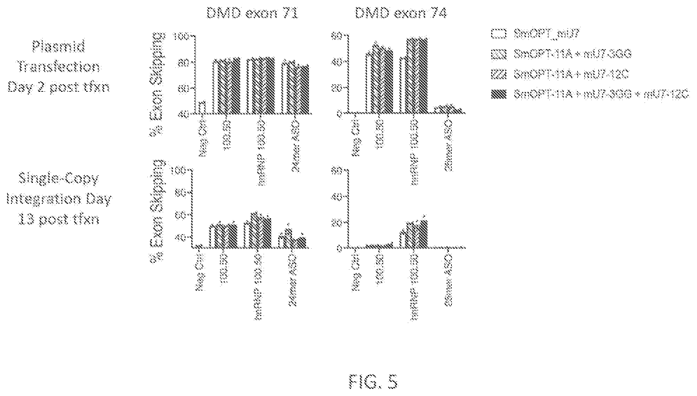

Representative SmOPT U7 hairpin variant combinations (DNA sequence SEQ ID NO: 49-SEQ ID NO: 51 or RNA sequence SEQ ID NO: 60-SEQ ID NO: 62) were then tested by individual transfections against gene targets for exon skipping. FIG. 5 shows the percent of exon skipping of the targeted transcripts by the indicated guide RNA. 293T cells were transfected with the respective plasmid constructs. Successful single-copy integrations were identified by fluorescence expression and selected for. Exon skipping efficacy for each guide RNA was measured 2 or 13 days after transfection.

The SmOPT U7 hairpin variants were appended to two antisense guide RNAs targeting the splice acceptor site for DMD exon 71 (SEQ ID NO: 78) or DMD exon 74 (SEQ ID NO: 79), with or without a 5′ hnRNPA1 domain (SEQ ID NO: 71). In addition, the SmOPT U7 hairpin variants were appended to shorter ASO sequences for DMD exon 71 or 74 exon skipping irrespective of ADAR editing (SEQ ID NO: 80, SEQ ID NO: 81). For each antisense sequence, the new SmOPT U7 hairpin variant showed the same or increased exon skipping compared to the original SmOPT mU7 hairpin sequence.

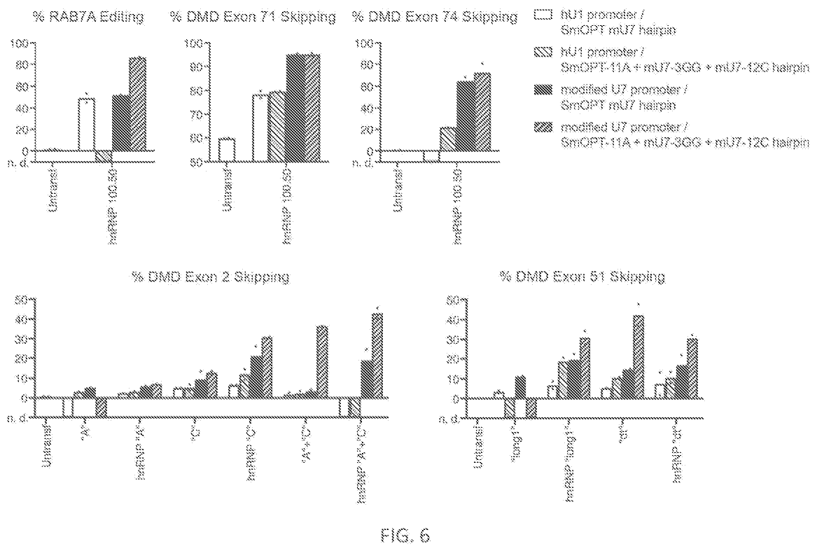

The SmOPT U7 hairpin variants were also tested in human RD rhabdomyosarcoma cells (CCL-136). FIG. 6 shows the percent of RAB7A editing or DMD exon skipping by the indicated guide RNA. RD cells were transfected with plasmid constructs expressing the antisense guide RNA from a human U1 promoter or a modified U7 promoter, along with a plasmid expressing piggybac transposase for random integration into the genome. Successful integrations were identified by fluorescence expression and selected for. Cells were subsequently differentiated for 10 days into myocytes to express the full-length DMD Dp427m muscle isoform. Then, RAB7A editing or DMD exon skipping was measured using droplet digital PCR. Untransfected RD cells after 10 days of myocyte differentiation were used as a negative control.

The original SmOPT and mU7 hairpin (SEQ ID NO: 2 and 3) or the SmOPT-11A with mU7-3GG-12C hairpin variant (SEQ ID NO: 51) were appended to antisense guide RNAs targeting the RAB7A 3′UTR (SEQ ID NO: 18), or DMD exon 71 (SEQ ID NO: 78) or DMD exon 74 (SEQ ID NO: 79), both with a 5′ hnRNPA1 domain (SEQ ID NO: 71). As in the 293T cells, for each antisense sequence, the new SmOPT U7 hairpin variant showed the same or increased RAB7A editing or DMD exon skipping compared to the original SmOPT mU7 hairpin sequence.

Existing antisense oligonucleotides possessing the original SmOPT U7 hairpin sequence are currently being used for exon skipping therapies; they act by physically masking Intronic and Exonic Splice Enhancer sequences, unrelated to ADAR editing. To demonstrate that the SmOPT U7 hairpin variants can also improve activity in this capacity, we tested antisense oligonucleotide sequences targeting clinically-relevant DMD exons. For DMD exon 2 skipping, antisense sequences “A” (SEQ ID NO: 100) and “C” (SEQ ID NO: 101) are currently being used in scAAV9.U7.ACCA for clinical trial NCT04240314. We also tested a longer antisense sequence, which encompasses both “A” and “C” (SEQ ID NO: 102), and covers the entirety of DMD exon 2. For DMD exon 51 skipping, we tested antisense sequences “long1” (SEQ ID NO: 103) and “dt” (SEQ ID NO: 104); “dt” is notable since it anneals to two non-contiguous sections of DMD exon 51. These antisense sequences were tested with or without a 5′ hnRNPA1 domain (SEQ ID NO: 71), and either the original SmOPT and mU7 hairpin (SEQ ID NO: 2 and 3) or the SmOPT-11A with mU7-3GG-12C hairpin variant (SEQ ID NO: 51). For each antisense oligonucleotide, the new SmOPT U7 hairpin outperformed the original SmOPT mU7 hairpin sequence. Furthermore, the additive combination of a 5′ hnRNPA1 sequence, the new SmOPT U7 hairpin, and modified U7 promoter increased the exon skipping efficiency of the published antisense oligonucleotides by 10-fold, for both DMD exon 2 and 51. This demonstrates that the new SmOPT U7 hairpin variants can increase the activity of antisense oligonucleotide sequences for exon skipping via covering as well as guide RNAs for ADAR editing.

While preferred embodiments of the present disclosure have been shown and described herein, such embodiments are provided by way of example only. Numerous variations, changes, and substitutions are encompassed without departing from the disclosure. It should be understood that various alternatives to the embodiments of the disclosure described herein can be employed in practicing the disclosure. It can be intended that the following claims define the scope of the disclosure and that methods and structures within the scope of these claims and their equivalents be covered thereby.

1.-110. (canceled)

111. An engineered RNA, comprising:
(a) a targeting sequence with complementarity to a target RNA;
(b) an RNA element that comprises:
(i) an engineered SmOPT variant sequence having at least 80% identity to SEQ ID NO: 87, SEQ ID NO: 84, SEQ ID NO: 85, or SEQ ID NO: 86; and
(ii) an engineered U7 hairpin variant sequence having SEQ at least 80% identity to SEQ ID NO: 96, SEQ ID NO: 90, SEQ ID NO: 91, SEQ ID NO: 92, SEQ ID NO: 93, SEQ ID NO: 94, SEQ ID NO: 95, SEQ ID NO: 97, SEQ ID NO: 98, or SEQ ID NO: 99.

112. The engineered RNA of claim 111, wherein the engineered SmOPT variant sequence comprises the sequence of SEQ ID NO: 87, SEQ ID NO: 84, SEQ ID NO: 85, or SEQ ID NO: 86.

113. The engineered RNA of claim 111, wherein the engineered U7 hairpin variant sequence comprises the sequence of SEQ ID NO: 96, SEQ ID NO: 90, SEQ ID NO: 91, SEQ ID NO: 92, SEQ ID NO: 93, SEQ ID NO: 94, SEQ ID NO: 95, SEQ ID NO: 97, SEQ ID NO: 98, or SEQ ID NO: 99.

114. The engineered RNA of claim 111, wherein the targeting sequence, upon hybridization to the target RNA, forms a guide-target RNA scaffold comprising a structural feature selected from the group consisting of a mismatch, a bulge, an internal loop, a hairpin, and any combination thereof, wherein the structural feature substantially forms upon hybridization to the target RNA, and wherein the structural feature is not present within an engineered guide RNA prior to the hybridization of the engineered guide RNA to the target RNA.

115. The engineered RNA of claim 111, further comprising a terminator.

116. The engineered RNA of claim 115, wherein the terminator is a U7 box terminator or a truncated terminator.

117. The engineered RNA of claim 111, wherein the RNA element comprises SEQ ID NO: 62, SEQ ID NO: 60, SEQ ID NO: 61, SEQ ID NO: 63; SEQ ID NO: 67, SEQ ID NO: 68, SEQ ID NO: 70.

118. The engineered RNA of claim 111, wherein the target RNA is DUX4, MAPT, SNCA, ABCA4, ALAS1, APP, ATP7B, CFTR, DMD, DMPK, GAPDH, GBA, HEXA, HFE, LIPA, LRRK2, PCSK9 start site, PINK1, PMP22, SERPINA1, SCNN1A start site, SOD1, a fragment of any one of these, or combination thereof.

119. The engineered RNA of claim 118, wherein the target RNA is SNCA, and wherein the SNCA comprises a mutation for RNA editing selected from the group consisting of: a translation initiation site (TIS) AUG to GTG in Codon 1, a TIS AUG in Codon 5, an AUG at position 265 in Exon 2, and any combination thereof.

120. The engineered RNA of claim 119, wherein the engineered RNA is a circularized engineered RNA.

121. A polynucleotide encoding an engineered RNA wherein the engineered RNA comprises:
(a) a targeting sequence with complementarity to a target RNA;
(b) an RNA element that comprises:
(iii) an engineered SmOPT variant sequence having at least 80% identity to SEQ ID NO: 87, SEQ ID NO: 84, SEQ ID NO: 85, or SEQ ID NO: 86; and
(iv) an engineered U7 hairpin variant sequence having SEQ at least 80% identity to SEQ ID NO: 96, SEQ ID NO: 90, SEQ ID NO: 91, SEQ ID NO: 92, SEQ ID NO: 93, SEQ ID NO: 94, SEQ ID NO: 95, SEQ ID NO: 97, SEQ ID NO: 98, or SEQ ID NO: 99.

122. The polynucleotide of claim 121, wherein the engineered SmOPT variant sequence comprises the sequence of SEQ ID NO: 87, SEQ ID NO: 84, SEQ ID NO: 85, or SEQ ID NO: 86.

123. The polynucleotide of claim 121, wherein the engineered U7 hairpin variant sequence comprises the sequence of SEQ ID NO: 96, SEQ ID NO: 90, SEQ ID NO: 91, SEQ ID NO: 92, SEQ ID NO: 93, SEQ ID NO: 94, SEQ ID NO: 95, SEQ ID NO: 97, SEQ ID NO: 98, or SEQ ID NO: 99.

124. The polynucleotide of claim 121, comprising the sequence of SEQ ID NO: 51, SEQ ID NO: 49, SEQ ID NO: 50, SEQ ID NO: 52, SEQ ID NO: 56, SEQ ID NO: 57, or SEQ ID NO: 59.

125. The polynucleotide of claim 121, wherein the polynucleotide is operably linked to an RNA polymerase II-type promoter.

126. The polynucleotide of claim 125, wherein the RNA polymerase II-type promoter is a U1 promoter, a U6 promoter, a U7 promoter, or a combination thereof.

127. A delivery vehicle comprising the engineered RNA of claim 111.

128. A delivery vehicle comprising the polynucleotide of claim 121.

129. The delivery vehicle of claim 128, wherein the delivery vehicle is a viral vector, wherein the viral vector is an adeno-associated viral (AAV) vector or derivative thereof.

130. The delivery vehicle of claim 129, wherein the AAV vector, derivative thereof, or a hybrid of any of these is selected from a group consisting of: AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAVhu68, a derivative of any of these, and a hybrid of any of these.

131. The delivery vehicle of claim 130, wherein the AAV vector or derivative thereof is selected from a group consisting of: a recombinant AAV (rAAV) vector, a hybrid AAV vector, a chimeric AAV vector, a self-complementary AAV (scAAV) vector, and any combination thereof.

132. A pharmaceutical composition, comprising the polynucleotide of claim 121 and a pharmaceutically acceptable: excipient, diluent, or carrier.

133. A method of treating a disease or condition in a subject, the method comprising: administering to the subject an effective amount of the pharmaceutical composition of claim 132 to treat the disease or condition in the subject.

134. The method of claim 133, wherein the disease or condition is selected from the group consisting of: a neurodegenerative disease or disorder, a muscular disease or disorder, a metabolic disease or disorder, an ocular disease or disorder, a liver disease or disorder, a cancer, and any combination thereof.

135. The method of claim 133, wherein the disease or condition is selected from the group consisting of: Duchenne's Muscular Dystrophy (DMD), Becker muscular dystrophy, myotonic dystrophy, Facioscapulohumeral muscular dystrophy, Rett's syndrome, Charcot-Marie-Tooth disease, Alzheimer's disease, a tauopathy, Parkinson's disease, alpha-1 antitrypsin deficiency, cystic fibrosis-like disease, Wilson disease, and Stargardt's disease.

136. The method of claim 133, wherein the disease or condition is associated with a mutation in a gene, or RNA encoded by DUX4, MAPT, SNCA, ABCA4, ALAS1, APP, ATP7B, CFTR, DMD, DMPK, GAPDH, GBA, HEXA, HFE, LIPA, LRRK2, PCSK9 start site, PINK1, PMP22, SERPINA1, SCNN1A start site, SOD1, a fragment of any one of these, or combination thereof.


SEQUENCE LISTING (114 sequences)

SEQ ID NO: 1 - RNA, 11 nt, synthetic construct
  source 1..11: mol_type = other RNA
aatttgtskag

SEQ ID NO: 2 - RNA, 11 nt, synthetic construct
  source 1..11: mol_type = other RNA
aatttttggag

SEQ ID NO: 3 - RNA, 31 nt, Mus sp.
  source 1..31: mol_type = genomic RNA
caggttttctgacttcggtcggaaaacccct

SEQ ID NO: 4 - RNA, 32 nt, Homo sapiens
  source 1..32: mol_type = genomic RNA
taggctttctggctttttaccggaaagcccct

SEQ ID NO: 5-7 - skipped (3 entries)

SEQ ID NO: 8 - RNA, 32 nt, synthetic construct
  source 1..32: mol_type = other RNA
  misc_difference 1: note = a, u, g, or c
  misc_difference 5: note = a, u, g, or c
  misc_difference 12: note = a, u, g, or c
  misc_difference 16: note = a, u, g, or c
  misc_difference 17: note = a, u, g, or c
  misc_difference 18: note = a, u, g, or c
  misc_difference 19: note = a, u, g, or c
  misc_difference 21: note = a, u, g, or c
  misc_difference 23: note = a, u, g, or c
  misc_difference 27: note = a, u, g, or c
  misc_difference 31: note = a, u, g, or c
  misc_difference 32: note = a, u, g, or c
naggntttctgncttnnnncngnaaancccnn

SEQ ID NO: 9 - RNA, 45 nt, synthetic construct
  source 1..45: mol_type = other RNA
gtggaatagtataacaatatgctaaatgttgttatagtatcccac

SEQ ID NO: 10 - DNA, 393 bp, Homo sapiens
  source 1..393: mol_type = genomic DNA
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 11 - DNA, 257 bp, Mus sp.
  source 1..257: mol_type = genomic DNA
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 12 - DNA, 263 bp, Homo sapiens
  source 1..263: mol_type = genomic DNA
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 13 - DNA, 107 bp, Mus sp.
  source 1..107: mol_type = genomic DNA
cccaatttcactggtctacaatgaaagcaaaacagttctcttccccgctccccggtgtgtgagaggggctttgatccttctctggtttcctaggaaacgcgtatgtg

SEQ ID NO: 14 - DNA, 95 bp, Homo sapiens
  source 1..95: mol_type = genomic DNA
cttatgatgtttgttgccaatgatagattgttttcactgtgcaaaaattatgggtagttttggtggtcttgatgcagttgtaagcttggggtatg

SEQ ID NO: 15 - DNA, 15 bp, Mus sp.
  source 1..15: mol_type = genomic DNA
gtctacaatgaaagc

SEQ ID NO: 16 - DNA, 21 bp, Homo sapiens
  source 1..21: mol_type = genomic DNA
tgtttgttgccaatgatagat

SEQ ID NO: 17 - DNA, 100 bp, synthetic construct
  source 1..100: mol_type = other DNA
aacatcgtagattgaagccacaaaatccacagcacacaaagaccctgccaccatgtattcacttcagtgaaagggaagcaccgaaatgctgagtgggggc

SEQ ID NO: 18 - DNA, 120 bp, synthetic construct
  source 1..120: mol_type = other DNA
tatgatagggacttagggtgtgataaaaggcgtacataattcttgtgtctactgtacagaatactgccgccagctggatttcccaattctgagtaacactctgcaatccaaacagggttc

SEQ ID NO: 19 - DNA, 115 bp, synthetic construct
  source 1..115: mol_type = other DNA
tggcagtgatggcatggactgtggtcatgagtccttccacgatacgttcgttgtcatggatgaccttggccaggggtgccaagcacaacgtggtgcaggaggcattgctgatgat

SEQ ID NO: 20 - RNA, 11 nt, synthetic construct
  source 1..11: mol_type = other RNA
aatttgtggag

SEQ ID NO: 21 - RNA, 11 nt, synthetic construct
  source 1..11: mol_type = other RNA
aatttgtctag

SEQ ID NO: 22 - DNA, 11 bp, synthetic construct
  source 1..11: mol_type = other DNA
aatttttggag

SEQ ID NO: 23 - DNA, 11 bp, synthetic construct
  source 1..11: mol_type = other DNA
aatttgtggag

SEQ ID NO: 24 - DNA, 11 bp, synthetic construct
  source 1..11: mol_type = other DNA
aatttgtctag

SEQ ID NO: 25 - DNA, 13 bp, synthetic construct
  source 1..13: mol_type = other DNA
caggttttctgac

SEQ ID NO: 26 - RNA, 13 nt, synthetic construct
  source 1..13: mol_type = other RNA
caggttttctgac

SEQ ID NO: 27 - DNA, 12 bp, synthetic construct
  source 1..12: mol_type = other DNA
taggctttctgg

SEQ ID NO: 28 - RNA, 12 nt, synthetic construct
  source 1..12: mol_type = other RNA
taggctttctgg

SEQ ID NO: 29 - DNA, 43 bp, synthetic construct
  source 1..43: mol_type = other DNA
taatttttggagcaggttttctgacttcggtcggaaaacccct

SEQ ID NO: 30 - DNA, 42 bp, synthetic construct
  source 1..42: mol_type = other DNA
aatttttggaacaggttttctgacttcggtcggaaaacccct

SEQ ID NO: 31 - DNA, 42 bp, synthetic construct
  source 1..42: mol_type = other DNA
aatttttggaccaggttttctgacttcggtcggaaaacccct

SEQ ID NO: 32 - DNA, 42 bp, synthetic construct
  source 1..42: mol_type = other DNA
aatttttggatcaggttttctgacttcggtcggaaaacccct

SEQ ID NO: 33 - DNA, 42 bp, synthetic construct
  source 1..42: mol_type = other DNA
aatttttggagctggttttctgacttcggtcggaaaacccct

SEQ ID NO: 34 - DNA, 44 bp, synthetic construct
  source 1..44: mol_type = other DNA
aatttttggagcagggttttctgacttcggtcggaaaaccccct

SEQ ID NO: 35 - DNA, 42 bp, synthetic construct
  source 1..42: mol_type = other DNA
aatttttggagcaggatttctgacttcggtcggaaatcccct

SEQ ID NO: 36 - DNA, 42 bp, synthetic construct
  source 1..42: mol_type = other DNA
aatttttggagcaggtcttctgacttcggtcggaagacccct

SEQ ID NO: 37 - DNA, 42 bp, synthetic construct
  source 1..42: mol_type = other DNA
aatttttggagcaggttttcagacttcggtctgaaaacccct

SEQ ID NO: 38 - DNA, 42 bp, synthetic construct
  source 1..42: mol_type = other DNA
aatttttggagcaggttttctgccttcgggcggaaaacccct

SEQ ID NO: 39 - RNA, 43 nt, synthetic construct
  source 1..43: mol_type = other RNA
taatttttggagcaggttttctgacttcggtcggaaaacccct

SEQ ID NO: 40 - RNA, 42 nt, synthetic construct
  source 1..42: mol_type = other RNA
aatttttggaacaggttttctgacttcggtcggaaaacccct

SEQ ID NO: 41 - RNA, 42 nt, synthetic construct
  source 1..42: mol_type = other RNA
aatttttggaccaggttttctgacttcggtcggaaaacccct

SEQ ID NO: 42 - RNA, 42 nt, synthetic construct
  source 1..42: mol_type = other RNA
aatttttggatcaggttttctgacttcggtcggaaaacccct

SEQ ID NO: 43 - RNA, 42 nt, synthetic construct
  source 1..42: mol_type = other RNA
aatttttggagctggttttctgacttcggtcggaaaacccct

SEQ ID NO: 44 - RNA, 44 nt, synthetic construct
  source 1..44: mol_type = other RNA
aatttttggagcagggttttctgacttcggtcggaaaaccccct

SEQ ID NO: 45 - RNA, 42 nt, synthetic construct
  source 1..42: mol_type = other RNA
aatttttggagcaggatttctgacttcggtcggaaatcccct

SEQ ID NO: 46 - RNA, 42 nt, synthetic construct
  source 1..42: mol_type = other RNA
aatttttggagcaggtcttctgacttcggtcggaagacccct

SEQ ID NO: 47 - RNA, 42 nt, synthetic construct
  source 1..42: mol_type = other RNA
aatttttggagcaggttttcagacttcggtctgaaaacccct

SEQ ID NO: 48 - RNA, 42 nt, synthetic construct
  source 1..42: mol_type = other RNA
aatttttggagcaggttttctgccttcgggcggaaaacccct

SEQ ID NO: 49 - DNA, 44 bp, synthetic construct
  source 1..44: mol_type = other DNA
aatttttggaacagggttttctgacttcggtcggaaaaccccct

SEQ ID NO: 50 - DNA, 42 bp, synthetic construct
  source 1..42: mol_type = other DNA
aatttttggaacaggttttctgccttcgggcggaaaacccct

SEQ ID NO: 51 - DNA, 44 bp, synthetic construct
  source 1..44: mol_type = other DNA
aatttttggaacagggttttctgccttcgggcggaaaaccccct

SEQ ID NO: 52 - DNA, 42 bp, synthetic construct
  source 1..42: mol_type = other DNA
aatttttggaacaggttttcagacttcggtctgaaaacccct

SEQ ID NO: 53 - DNA, 44 bp, synthetic construct
  source 1..44: mol_type = other DNA
aatttttggagcagggttttcagacttcggtctgaaaaccccct

SEQ ID NO: 54 - DNA, 44 bp, synthetic construct
  source 1..44: mol_type = other DNA
aatttttggagcagggttttctgccttcgggcggaaaaccccct

SEQ ID NO: 55 - DNA, 42 bp, synthetic construct
  source 1..42: mol_type = other DNA
aatttttggagcaggttttcagccttcgggctgaaaacccct

SEQ ID NO: 56 - DNA, 44 bp, synthetic construct
  source 1..44: mol_type = other DNA
aatttttggaacagggttttcagacttcggtctgaaaaccccct

SEQ ID NO: 57 - DNA, 42 bp, synthetic construct
  source 1..42: mol_type = other DNA
aatttttggaacaggttttcagccttcgggctgaaaacccct

SEQ ID NO: 58 - DNA, 44 bp, synthetic construct
  source 1..44: mol_type = other DNA
aatttttggagcagggttttcagccttcgggctgaaaaccccct

SEQ ID NO: 59 - DNA, 44 bp, synthetic construct
  source 1..44: mol_type = other DNA
aatttttggaacagggttttcagccttcgggctgaaaaccccct

SEQ ID NO: 60 - RNA, 44 nt, synthetic construct
  source 1..44: mol_type = other RNA
aatttttggaacagggttttctgacttcggtcggaaaaccccct

SEQ ID NO: 61 - RNA, 42 nt, synthetic construct
  source 1..42: mol_type = other RNA
aatttttggaacaggttttctgccttcgggcggaaaacccct

SEQ ID NO: 62 - RNA, 44 nt, synthetic construct
  source 1..44: mol_type = other RNA
aatttttggaacagggttttctgccttcgggcggaaaaccccct

SEQ ID NO: 63 - RNA, 42 nt, synthetic construct
  source 1..42: mol_type = other RNA
aatttttggaacaggttttcagacttcggtctgaaaacccct

SEQ ID NO: 64 - RNA, 44 nt, synthetic construct
  source 1..44: mol_type = other RNA
aatttttggagcagggttttcagacttcggtctgaaaaccccct

SEQ ID NO: 65 - RNA, 44 nt, synthetic construct
  source 1..44: mol_type = other RNA
aatttttggagcagggttttctgccttcgggcggaaaaccccct

SEQ ID NO: 66 - RNA, 42 nt, synthetic construct
  source 1..42: mol_type = other RNA
aatttttggagcaggttttcagccttcgggctgaaaacccct

SEQ ID NO: 67 - RNA, 44 nt, synthetic construct
  source 1..44: mol_type = other RNA
aatttttggaacagggttttcagacttcggtctgaaaaccccct

SEQ ID NO: 68 - RNA, 42 nt, synthetic construct
  source 1..42: mol_type = other RNA
aatttttggaacaggttttcagccttcgggctgaaaacccct

SEQ ID NO: 69 - RNA, 44 nt, synthetic construct
  source 1..44: mol_type = other RNA
aatttttggagcagggttttcagccttcgggctgaaaaccccct

SEQ ID NO: 70 - RNA, 44 nt, synthetic construct
  source 1..44: mol_type = other RNA
aatttttggaacagggttttcagccttcgggctgaaaaccccct

SEQ ID NO: 71 - DNA, 20 bp, synthetic construct
  source 1..20: mol_type = other DNA
tatgatagggacttagggtg

SEQ ID NO: 72 - DNA, 80 bp, synthetic construct
  source 1..80: mol_type = other DNA
gtacataattcttgtgtctactgtacagaatactgccgccagctggatttcccaattctgagtaacactctgcaatccaa

SEQ ID NO: 73 - DNA, 80 bp, synthetic construct
  source 1..80: mol_type = other DNA
gattgaagccacaaaatccacagcacacaaagaccctgccaccatgtattcacttcagtgaaagggaagcaccgaaatgc

SEQ ID NO: 74 - DNA, 80 bp, synthetic construct
  source 1..80: mol_type = other DNA
gataccaaagttgtcatggatgaccttggccaggggtgccaagcagttggtggtgcaggaggcattgctgatgatcttga

SEQ ID NO: 75 - DNA, 100 bp, synthetic construct
  source 1..100: mol_type = other DNA
gccctgcactgggccgtcgcccttcagcacgcacacggccttcgtcgccacaactcgctaggccacgccgaggtcctggttccgaggactgcaacggaaa

SEQ ID NO: 76 - DNA, 100 bp, synthetic construct
  source 1..100: mol_type = other DNA
gattgtcccaagatgacatcagctcattctcacagcccagcgagggcacctactcgtgtaatgcgtggccacaggtcatcacctgtcctgtggccctggc

SEQ ID NO: 77 - DNA, 100 bp, synthetic construct
  source 1..100: mol_type = other DNA
agtctaaaggttgtgccagtgcaatcggcatggtatgaagtacttcgtccaggaggaccagggcccggtgtaagtgaatttcaatccagcaaggtgtttc

SEQ ID NO: 78 - DNA, 100 bp, synthetic construct
  source 1..100: mol_type = other DNA
aagtactcacgcagaatctactggccagaagttgatcagagtaacgggaccgcaaaacaaaaaatgaggtggtgaaggagacacacgcaaactcagccgc

SEQ ID NO: 79 - DNA, 100 bp, synthetic construct
  source 1..100: mol_type = other DNA
ctggttcaaactttggcagtaatgctggattaacaaatgttcatcatctccggaaaataaaatcaaaggttttggttttttcccccccttattttgcttt

SEQ ID NO: 80 - DNA, 24 bp, synthetic construct
  source 1..24: mol_type = other DNA
aagttgatcagtgtaacgggactg

SEQ ID NO: 81 - DNA, 25 bp, synthetic construct
  source 1..25: mol_type = other DNA
cgaggctggctcaggggggagtcct

SEQ ID NO: 82 - RNA, 12 nt, synthetic construct
  source 1..12: mol_type = other RNA
  misc_difference 1: note = May be absent
  misc_difference 10: note = g, a, c or u
  misc_difference 12: note = g, a, c or u
taatttttgnan

SEQ ID NO: 83 - RNA, 11 nt, synthetic construct
  source 1..11: mol_type = other RNA
aatttttghah

SEQ ID NO: 84 - RNA, 12 nt, synthetic construct
  source 1..12: mol_type = other RNA
taatttttggag

SEQ ID NO: 85 - RNA, 11 nt, synthetic construct
  source 1..11: mol_type = other RNA
aatttttggac

SEQ ID NO: 86 - RNA, 11 nt, synthetic construct
  source 1..11: mol_type = other RNA
aatttttggat

SEQ ID NO: 87 - RNA, 11 nt, synthetic construct
  source 1..11: mol_type = other RNA
aatttttggaa

SEQ ID NO: 88 - RNA, 33 nt, synthetic construct
  source 1..33: mol_type = other RNA
  misc_difference 5: note = May be absent
  misc_difference 32: note = May be absent
cwgggwyttcwgmcttcggkckgaarwcccctt

SEQ ID NO: 89 - RNA, 33 nt, synthetic construct
  source 1..33: mol_type = other RNA
  misc_difference 5: note = May be absent
  misc_difference 32: note = May be absent
cagggttttctgmcttcggkcggaaaacccctt

SEQ ID NO: 90 - RNA, 31 nt, synthetic construct
  source 1..31: mol_type = other RNA
ctggttttctgacttcggtcggaaaacccct

SEQ ID NO: 91 - RNA, 33 nt, synthetic construct
  source 1..33: mol_type = other RNA
cagggttttctgacttcggtcggaaaaccccct

SEQ ID NO: 92 - RNA, 31 nt, synthetic construct
  source 1..31: mol_type = other RNA
caggatttctgacttcggtcggaaatcccct

SEQ ID NO: 93 - RNA, 31 nt, synthetic construct
  source 1..31: mol_type = other RNA
caggtcttctgacttcggtcggaagacccct

SEQ ID NO: 94 - RNA, 31 nt, synthetic construct
  source 1..31: mol_type = other RNA
caggttttcagacttcggtctgaaaacccct

SEQ ID NO: 95 - RNA, 31 nt, synthetic construct
  source 1..31: mol_type = other RNA
caggttttctgccttcgggcggaaaacccct

SEQ ID NO: 96 - RNA, 33 nt, synthetic construct
  source 1..33: mol_type = other RNA
cagggttttctgccttcgggcggaaaaccccct

SEQ ID NO: 97 - RNA, 33 nt, synthetic construct
  source 1..33: mol_type = other RNA
cagggttttcagacttcggtctgaaaaccccct

SEQ ID NO: 98 - RNA, 31 nt, synthetic construct
  source 1..31: mol_type = other RNA
caggttttcagccttcgggctgaaaacccct

SEQ ID NO: 99 - RNA, 33 nt, synthetic construct
  source 1..33: mol_type = other RNA
cagggttttcagccttcgggctgaaaaccccct

SEQ ID NO: 100 - DNA, 31 bp, synthetic construct
  source 1..31: mol_type = other DNA
gttttcttttgaacatcttctctttcatcta

SEQ ID NO: 101 - DNA, 23 bp, synthetic construct
  source 1..23: mol_type = other DNA
attcttaccttagaaaattgtgc

SEQ ID NO: 102 - DNA, 75 bp, synthetic construct
  source 1..75: mol_type = other DNA
ccattcttaccttagaaaattgtgcatttacccattttgtgaatgttttcttttgaacatcttctctttcatcta

SEQ ID NO: 103 - DNA, 45 bp, synthetic construct
  source 1..45: mol_type = other DNA
gcaggtacctccaacatcaaggaagatggcatttctagtttggag

SEQ ID NO: 104 - DNA, 43 bp, synthetic construct
  source 1..43: mol_type = other DNA
cctctgtgattttataacttgattcaaggaagatggcatttct

SEQ ID NO: 105 - DNA, 56 bp, synthetic construct
  source 1..56: mol_type = other DNA
gccatcagtcgccggtcccaagcccggataaaatgggagggggcgggaaaccgcct

SEQ ID NO: 106 - RNA, 56 nt, synthetic construct
  source 1..56: mol_type = other RNA
gccatcagtcgccggtcccaagcccggataaaatgggagggggcgggaaaccgcct

SEQ ID NO: 107 - DNA, 55 bp, synthetic construct
  source 1..55: mol_type = other DNA
aacactgccaatgccggtcccaagcccggataaaagtggagggtacagtccacgc

SEQ ID NO: 108 - RNA, 55 nt, synthetic construct
  source 1..55: mol_type = other RNA
aacactgccaatgccggtcccaagcccggataaaagtggagggtacagtccacgc

SEQ ID NO: 109 - DNA, 21 bp, synthetic construct
  source 1..21: mol_type = other DNA
aaccatgccgactgatggcag

SEQ ID NO: 110 - DNA, 26 bp, synthetic construct
  source 1..26: mol_type = other DNA
gatgtcaggtgcggctgactaccgtc

SEQ ID NO: 111 - RNA, 21 nt, synthetic construct
  source 1..21: mol_type = other RNA
aaccatgccgactgatggcag

SEQ ID NO: 112 - RNA, 26 nt, synthetic construct
  source 1..26: mol_type = other RNA
gatgtcaggtgcggctgactaccgtc

SEQ ID NO: 113 - RNA, 12 nt, synthetic construct
  source 1..12: mol_type = other RNA
  misc_difference 1: note = May be absent
  misc_difference 10: note = g, a, c or u
  misc_difference 12: note = g, a, c or u
waatttttgnan

SEQ ID NO: 114 - RNA, 13 nt, synthetic construct
  source 1..13: mol_type = other RNA
  misc_difference 1: note = May be absent
  misc_difference 10: note = g, a, c or u
  misc_difference 13: note = g, a, c or u
taatttttgngan